(19) United States
(12) Patent Application Publication
(10) Pub. No.: US 2005/0020594 A1
(43) Pub. Date: Jan. 27, 2005

(54) Substituted pyridazinones

(75) Inventors: Michael Hepperle, Boston, MA (US); Kevin Jerome, Maryland Heights, MO (US); John Walker, Maryland Heights, MO (US); Shaun Selness, Chesterfield, MO (US); Rajesh Devraj, Ballwin, MO (US)

Correspondence Address: PHARMACIA CORPORATION;GLOBAL PATENT DEPARTMENT, POST OFFICE BOX 1027, ST. LOUIS, MO, 63006, US

(73) Assignee: Pharmacia Corporation

(21) Appl. No.: 10/893,073

(22) Filed: Jul. 16, 2004

Related U.S. Application Data
(60) Provisional application No. 60/488,378, filed on Jul. 18, 2003.

(51) Int. Cl. A61K 31/501; C 07D 4 3/02

(52) U.S. Cl. 514/247; 514/252.01; 544/238; 544/239

(57) ABSTRACT

Disclosed are substituted pyridazinones that are useful for treating diseases and conditions caused or exacerbated by unregulated p38 MAP Kinase and/or TNF activity. Pharmaceutical composition containing the pyridazinone compounds, methods of preparing the compounds and methods of treatment using the compounds are also disclosed.

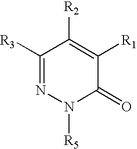

This application claims priority to U.S. Provisional application No. 60/488,378 filed Jul. 18, 2003.

### BACKGROUND OF THE INVENTION

1. Field of the Invention

The invention relates to substituted pyridazinones that are useful for treating diseases and conditions caused or exacerbated by unregulated p38 MAP Kinase activity. Pharmaceutical compositions containing the pyridazinone compounds, methods of preparing the compounds and methods of treatment using the compounds are also disclosed.

2. Description of the Related Art

Nearly all cell surface receptors use one or more of the mitogen-activated protein kinase (MAP kinase) cascades during signal transduction. MAP kinases are a family of proline-directed serine/threonine kinases that activate their substrates by dual phosphorylation. Four distinct subgroups of MAP kinases, p38 alpha, p38 beta p38 gamma, and p38 delta have been identified and each of these consists of a specific module of kinases that function downstream of an activating stimulus by phosphorylating and activating transcription factors (e.g. ATF2, CHOP and MEF2C) as well as other kinases (e.g. MAPKAP-2 and MAPKAP-3). One subgroup of the MAP kinases is the p38 MAP kinase cascade, which is activated by a variety of signals including proinflammatory cytokines such as tumor necrosis factor (TNF) and interleukin-1 (IL-1) as well as bacterial lipopolysaccharides, and environmental stress (e.g., osmotic shock and ultraviolet radiation). Upon activation, the p38 cascade leads to the induction of gene expression of several factors involved in inflammation and immunity including TNF, interleukin-6, granulocyte-macrophage colony stimulating factor (GM-CSF), and HIV long terminal repeat (Paul et al., Cell Signal., 1997, 9, 403-410). The products of the p38 phosphorylation inhibit or modulate the production of inflammatory cytokines, including TNF and IL-1, and cyclooxygenase-2, and also potentially block the effects of these cytokines on their target cells, which therefore inhibit or modulate inflammation.

p38 MAP kinases have also been shown to help prevent apoptosis during ischemia in cardiac myocytes, which suggests that p38 MAP kinase inhibitors can be used for treating ischemic heart disease, p38 MAP kinase is also required for T-cell HIV-1 replication and may be a useful target for AIDS therapy. p38 Pathway inhibitors have also been used to increase cancer cell sensitivity to cancer therapy.

TNF is a cytokine and a potent proinflammatory mediator implicated in inflammatory conditions such as arthritis, asthma, septic shock, non-insulin dependent diabetes mellitus, multiple sclerosis, asthma, and inflammatory bowel disease. TNF has also been implicated in viral infections, such as HIV, influenza virus, and herpes virus including herpes simplex virus type-1 (HSV-1), herpes simplex virus type-2 (HSV-2), cytomegalovirus (CMV), varicella-zoster virus (VZV), Epstein-Barr virus, human herpesvirus-6 (HHV-6), human herpesvirus-7 (HHV-7), human herpesvirus-8 (HHV-8), pseudorabies and rhinotracheitis, among others.

Excessive or unregulated TNF production has also been shown to produce elevated levels of IL-1. Inhibition of TNF, therefore, should reduce levels of IL-1 and ameliorate disease states caused by unregulated IL-1 synthesis. Such disease states include rheumatoid arthritis, rheumatoid spondylitis, osteoarthritis, gouty arthritis, sepsis, septic shock, endotoxic shock, gram negative sepsis, toxic shock syndrome, adult respiratory distress syndrome, cerebral malaria, chronic pulmonary inflammatory disease, silicosis, pulmonary sarcosis, bone resorption diseases, reperfusion injury, graft versus host reaction, alallograft rejections, fever and myalgias due to infection, cachexia secondary to infection or malignancy, cachexia secondary to acquired immune deficiency syndrome (AIDS), AIDS related complex (ARC), keloid formation, scar tissue formation, Crohn's disease, ulcerative colitis, and pyresis.

IL-1 has also been shown to mediate a variety of biological activities such as the activation of T-helper cells, induction of fever, stimulation of prostaglandin or collagenase production, neutrophil chemotaxis, and the suppression of plasma iron levels (Rev. Infect. Disease, 6, 51 (1984)). Elevated levels of IL-1 have also been implicated in mediating or exacerbating a number of disease states including rheumatoid arthritis, osteoarthritis, rheumatoid spondylitis, gouty arthritis, inflammatory bowel disease, adult respiratory distress syndrome (ARDS), psoriasis, Crohn's disease, ulcerative colitis, anaphylaxis, muscle degeneration, cachexia, Reiter's syndrome, type I and type II diabetes, bone resorption diseases, ischemia reperfusion injury, arteriosclerosis, brain trauma, multiple sclerosis, sepsis, septic shock, and toxic shock syndrome. Viruses sensitive to TNF inhibition, such as HIV-1, HIV-2, HIV-3, are also affected by IL-1 production. In rheumatoid arthritis, both IL-1 and TNF induce collagenase synthesis and ultimately lead to tissue destruction within arthritic joints (Lymphokine Cytokine Res. (11): 253-256, (1992) and Clin. Exp. Immunol. 989:244-250, (1992)).

IL-6 is another pro-inflammatory cytokine, which is associated with many conditions including inflammation.

Consequently, TNF, IL-1 and IL-6 affect a wide variety of cells and tissues and are important inflammatory mediators of a wide variety of disease states and conditions. The inhibition of these cytokines by inhibition or modulation of p38 alpha and/or p38 beta kinase is of benefit in controlling, reducing and alleviating many of these disease states and conditions. Therefore, the invention concerns finding small molecule inhibitors or modulators of p38 alpha and/or p38 beta kinase and the p38 alpha and/or p38 beta kinase pathway.

### SUMMARY OF THE INVENTION

In a broad aspect, the invention provides compounds of Formula I: 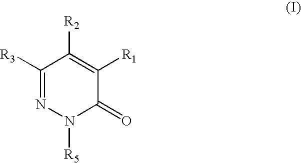
and pharmaceutically acceptable salt thereof, wherein
R₁ is H, halogen, NO₂, alkyl, carboxaldehyde, hydroxyalkyl, dihydroxyalkyl, arylalkoxy, arylalkyl, alkenyl, alkynyl, arylalkynyl, —CN, aryl, alkanoyl, alkoxy, alkoxyalkyl, haloalkyl, haloalkoxy, carboxyl, aryloxy(C₁-C₆)alkyl, or arylalkanoyl,
   wherein the aryl portion of arylalkoxy, arylalkyl, and arylalkanoyl is unsubstituted or substituted with 1, 2, 3, 4, or 5 groups that are independently halogen, C₁-C₄ alkyl, C₁-C₄ alkoxy, nitro, CN, haloalkyl, haloalkoxy or CO₂R;
   wherein the alkyl portion of the alkyl, hydroxyalkyl, dihydroxyalkyl, arylalkoxy, aryloxy(C₁-C₆)alkyl, arylalkyl, alkanoyl, alkoxy, alkoxyalkyl and arylalkanoyl groups is unsubstituted or substituted with 1, 2, or 3 groups that are independently halogen, C₁-C₄ alkoxy, C₁-C₄ alkoxycarbonyl, or C₃-C₇ cycloalkyl;
R₂ is H, OH, halogen, —OSO₂—(C₁-C₆)alkyl, —OSO₂-aryl, arylalkoxy, heteroarylalkoxy, aryloxy, arylthio, arylalkylthio, arylamino (C₁-C₆)alkyl, arylalkylamino, arylthioalkoxy, arylalkynyl, alkoxy, aryloxy(C₁-C₆)alkyl, alkyl, alkynyl, —OC(O)NH(CH₂)ₙaryl, —OC(O)N(alkyl)(CH₂)ₙaryl, alkoxyalkoxy, dialkylamino, alkyl, alkoxy, aryl, arylalkyl, heteroaryl, heteroarylalkyl, arylalkenyl, heterocycloalkyl, heterocycloalkylalkyl, alkoxyalkoxy, NR₈R₉, dialkylamino, or CO₂R, wherein
   n is 0, 1, 2, 3, 4, 5 or 6;
   each of which groups is unsubstituted or substituted with 1, 2, 3, 4, or 5 groups that are independently halogen, —(C₁-C₆)alkyl-N(R)—CO₂R₃₀, haloalkyl, heteroaryl, heteroarylalkyl, —(C₁-C₆alkyl)-C(O)—NR₆R₇, —NR₆R₇, R₆R₇N—(C₁-C₆ alkyl)-, —C(O)NR₆R₇, —(C₁-C₄ alkyl)-NRC(O)NR₁₆R₁₇, —(Cl—C₄)alkyl-OSO₂—(C₁-C₆)alkyl, haloalkoxy, alkyl, CN, hydroxyalkyl, dihydroxyalkyl, alkoxy, alkoxycarbonyl, phenyl, —SO₂-phenyl wherein the phenyl and —SO₂-phenyl groups are optionally substituted with 1, 2, or 3 groups that are independently halogen or NO₂, or —OC(O)NR₆R₇, wherein R₁₆ and R₁₇ are independently H or C₁-C₆ alkyl; or
      R₁₆, R₁₇ and the nitrogen to which they are attached form a morpholinyl ring;
      R₆ and R₇ are independently at each occurrence H, alkyl, hydroxyalkyl, dihydroxyalkyl, alkoxy, alkanoyl, arylalkyl, arylalkoxy, alkoxycarbonyl, —SO₂-alkyl, OH, alkoxy, alkoxyalkyl, arylalkoxycarbonyl, —(C₁-C₄)alkyl-CO₂-alkyl, heteroarylalkyl, or arylalkanoyl, wherein each is unsubstituted or substituted with 1, 2, or 3 groups that are independently, halogen, OH, SH, heterocycloalkyl, heterocycloalkylalkyl, C₃-C₇ cycloalkyl, alkoxy, NH₂, NH(alkyl), N(alkyl)(alkyl), —O-alkanoyl, alkyl, haloalkyl, carboxaldehyde, or haloalkoxy; or
      R₆, R₇, and the nitrogen to which they are attached form a morpholinyl, pyrrolidinyl, thiomorpholinyl, thiomorpholinyl S-oxide, thiomorpholinyl S,S-dioxide, piperidinyl, pyrrolidinyl, or piperazinyl ring which is optionally substituted with 1 or 2 groups that are independently C₁-C₄ alkyl, alkoxycarbonyl, C₁-C₄ alkoxy, hydroxyl, hydroxyalkyl, dihydroxyalkyl, or halogen;
      R at each occurrence is independently hydrogen or C₁-C₆ alkyl optionally substituted with 1 or 2 groups that are independently OH, SH, halogen, amino, monoalkylamino, dialkylamino or C₃-C₆ cycloalkyl;
      R₃₀ is C₁-C₆ alkyl optionally substituted with 1 or 2 groups that are independently OH, SH, halogen, amino, monoalkylamino, dialkylamino or C₃-C₆ cycloalkyl;
      each R₈ is independently hydrogen, alkyl, alkanoyl, arylalkyl and arylalkanoyl, wherein each of the above is optionally substituted with 1, 2, 3, 4, or 5 groups that are independently alkyl, alkoxy, alkoxycarbonyl, halogen, or haloalkyl;
      each R₉ is hydrogen, alkyl, alkanoyl, arylalkyl, cycloalkyl, arylcycloalkyl, cycloalkylalkyl, heterocycloalkylalkyl, arylheterocycloalkyl, alkenyl, heteroaryl, amino, aminoalkyl, monoalkylaminoalkyl, dialkylaminoalkyl, arylalkanoyl, —SO₂-phenyl, and aryl wherein each of the above is optionally substituted with 1, 2, 3, 4, or 5 groups that are independently alkyl, alkoxy, hydroxy, hydroxyalkyl, amino, —(CH₂)₀₋₄-COOR, alkoxycarbonyl, halogen, or haloalkyl;
R₃ is H, halogen, alkoxycarbonyl, arylalkoxycarbonyl, aryloxycarbonyl, arylalkyl, —OC(O)NH(CH₂)ₙaryl, arylalkoxy, —OC(O)N(alkyl)(CH₂)ₙaryl, aryloxy, arylthio, thioalkoxy, arylthioalkoxy, alkenyl, —COOR, hydroxyalkyl, arylalkylcarbonyl, arylalkoxyalkyl, —NR₆R₇, —C(O)NR₆R₇, NR₆R₇—(C₁-C₆)alkyl, or alkyl, wherein
   the aryl portion of arylalkoxycarbonyl, aryloxycarbonyl, arylalkyl, —OC(O)NH(CH₂)ₙaryl, arylalkoxy, —OC(O)N(alkyl)(CH₂)ₙaryl, arylalkoxyalkyl, and arylthioalkoxy, is unsubstituted or substituted with 1, 2, 3, 4, or 5 groups that are independently, halogen, alkoxy, alkyl, haloalkyl, or haloalkoxy,
      wherein n is 0, 1, 2, 3, 4, 5, or 6; and
R₅ is H, aryl, heteroaryl, arylalkyl, arylthioalkyl, alkyl optionally substituted with 1, 2, or 3 groups that are independently arylalkoxycarbonyl, —NR₈R₉, halogen, —C(O)NR₈R₉, alkoxycarbonyl, C₃-C₇ cycloalkyl, or alkanoyl, alkoxy, alkoxyalkyl optionally substituted with one trimethylsilyl group, amino, alkoxycarbonyl, hydroxyalkyl, dihydroxyalkyl, alkynyl, —SO₂-alkyl, alkoxy optionally substituted with one trimethylsilyl group, heterocycloalkylalkyl, cycloalkyl, cycloalkylalkyl, —alkyl-S-aryl, -alkyl-SO₂-aryl, heteroarylalkyl, heterocycloalkyl, heteroaryl, or alkenyl optionally substituted with alkoxycarbonyl, wherein
   each of the above is unsubstituted or substituted with 1, 2, 3, 4, or 5 groups that are independently alkyl, halogen, alkoxy, hydroxyalkyl, dihydroxyalkyl, arylalkoxy, thioalkoxy, alkoxycarbonyl, arylalkoxycarbonyl, CO₂R, CN, OH, hydroxyalkyl, dihydroxyalkyl, amidinooxime, —NR₆R₇, —NR₈R₉, R₆R₇N—(C₁-C₆ alkyl)-, carboxaldehyde, SO₂alkyl, —SO₂H, —SO₂NR₆R₇, alkanoyl wherein the alkyl portion is optionally substituted with OH, halogen or alkoxy, —C(O)NR₆R₇, —(C₁-C₄ alkyl)-C(O)NR₆R₇, amidino, haloalkyl, —(C₁-C₄ alkyl)-NR₁₅C(O)NR₁₆R₁₇, —(C₁-C₄ alkyl)-NR₁₅C(O)R₁₈, —O—CH₂—O, —O—CH₂CH₂—O—, or haloalkoxy; wherein
      R₁₅ is H or C₁-C₆ alkyl;
   R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl,
provided that no more than two of R₁, R₂, and R₅ are simultaneously hydrogen.

The invention also includes intermediates useful in making the compounds of the invention.

Compounds of the invention bind and/or interact with the p38 kinase and/or TNF enzymes. Preferably, they inhibit the activity of p38 kinase and/or TNF. They are therefore used in treating p38 or TNF mediated disorders.

In particular, they are useful for treating p38 alpha kinase mediated disorders.

The invention also includes pharmaceutical compositions comprising at least one compound of formula I and at least one pharmaceutically acceptable carrier, solvent, adjuvant or excipient.

The invention also includes methods of treating a TNF mediated disorder, a p38 kinase mediated disorder, inflammation and/or arthritis in a subject, the method comprising treating a subject having or susceptible to such disorder or condition with a therapeutically-effective amount of a compound of Formula I.

### DETAILED DESCRIPTION OF THE INVENTION

A preferred class of compounds of formula I are those wherein,
R₁ is H, bromo, chloro, iodo, or alkyl; and
R₂ is phenyl(C₁-C₆)alkoxy, phenyloxy, —S-phenyl, (C₁-C₆)alkoxy, NR₆R₇, H, OH, halogen, or thio(C₁-C₆)alkoxy;
   wherein each of the above is optionally substituted with 1, 2, or 3 groups that are independently halogen, hydroxyalkyl, alkoxy, or alkyl, wherein
   R₆ and R₇ at each occurrence are independently selected from H, alkyl, hydroxy (C₁-C₄)alkyl, phenylalkyl, (C₂-C₆)alkanoyl, (C₃-C₆)cycloalkyl optionally substituted with phenyl, phenyl, and tetrahydrofuryl(C₁-C₆)alkyl,
      wherein the phenyl groups are optionally substituted with 1, 2, 3, 4, or 5 groups that are independently halogen, alkyl, NH₂, monoalkylamino, dialkylamino, or alkoxy,
      wherein the alkyl portions of the above groups are optionally substituted with 1, 2, or 3 groups that are independently CO₂H, OH, hydroxy (C₁-C₄)alkyl, or alkoxycarbonyl.

Other preferred compounds are those wherein,
R₃ is H, —C(O)NR₆R₇, hydroxy(C₁-C₆)alkyl, —(C₁-C₄ alkyl)-NR₆R₇, alkoxyalkyl, CO₂H, phenyl(C₁-C₆)alkyl; and
R₅ is phenyl, or phenyl(C₁-C₆)alkyl each of which is optionally substituted with 1, 2, 3, 4, or 5 groups that are independently halogen, alkyl or alkoxy;
   R₆ and R₇ at each occurrence are independently selected from H, alkyl, hydroxy (C₁-C₄)alkyl, phenylalkyl, (C₂-C₆)alkanoyl, (C₃-C₆)cycloalkyl optionally substituted with phenyl, phenyl, and tetrahydrofuryl(C₁-C₆)alkyl,
      wherein the phenyl groups are optionally substituted with 1, 2, 3, 4, or 5 groups that are independently halogen, alkyl, NH₂, monoalkylamino, dialkylamino, or alkoxy,
      wherein the alkyl portions of the above groups are optionally substituted with 1, 2, or 3 groups that are independently CO₂H, OH, hydroxy (C₁-C₄)alkyl, or alkoxycarbonyl.

Still other preferred compounds are those wherein,
R₁ is H, bromo, chloro, iodo, or alkyl; and
R₂ is phenyl(C₁-C₆)alkoxy, phenyloxy, —S-phenyl, (C₁-C₆)alkoxy, pyridyl(C₁-C₆)alkoxy, NR₆R₇, H, OH, halogen or thio(C₁-C₆)alkoxy;
   wherein each of the above is optionally substituted with 1, 2, or 3 groups that are independently halogen, hydroxyalkyl, alkoxy, or alkyl, wherein
R₃ is H, —C(O)NR₆R₇, hydroxy(C₁-C₆)alkyl, —(C₁-C₄ alkyl)-NR₆R₇, alkoxyalkyl, CO₂H, phenyl(C₁-C₆)alkyl; and
R₅ is phenyl, or phenyl(C₁-C₆)alkyl each of which is optionally substituted with 1, 2, 3, 4, or 5 groups that are independently halogen, alkyl or alkoxy;
   R₆ and R₇ are independently at each occurrence selected from H, NH₂, alkyl, hydroxy (C₁-C₄)alkyl, phenylalkyl, (C₂-C₆)alkanoyl, (C₃-C₆)cycloalkyl optionally substituted with phenyl, phenyl, and tetrahydrofuryl(C₁-C₆)alkyl,
      wherein the phenyl groups are optionally substituted with 1, 2, 3, 4, or 5 groups that are independently halogen, alkyl, NH₂, monoalkylamino, dialkylamino, or alkoxy,
      wherein the alkyl portions of the above groups are optionally substituted with 1, 2, or 3 groups that are independently CO₂H, OH, hydroxy (C₁-C₄)alkyl, or alkoxycarbonyl.

Still other preferred compounds are compounds of formula I are those of formula Ia, wherein,
R₁ is H, bromo, chloro, or iodo;
R₂ is (C₁-C₆)alkoxy, benzyl, benzyloxy, phenethyloxy, phenpropyloxy, pyridyl(C₁-C₆)alkoxy, phenyloxy, or —S-phenyl each of which is optionally substituted with 1, 2, or 3 groups that are independently halogen, hydroxyalkyl, haloalkyl, alkoxy, or alkyl;
R₃ is H; and
R₅ is benzyl or phenyl, each of which is optionally substituted with 1, 2, or 3 groups that are independently halogen, alkyl or alkoxy.

Other preferred compounds of formula 1a are those wherein,
R₂ is pyridyl(C₁-C₄)alkoxy, which is optionally substituted with 1, 2, or 3 groups that are independently halogen, hydroxyalkyl, alkoxy, or alkyl.

More preferred compounds of formula 1a are compounds of formula Ib, wherein
R₁ is bromo or chloro; and
R₅ is benzyl, phenyl, or 2,6-disubstituted phenyl, wherein the substituents are independently halogen, alkyl or alkoxy.

Still more preferred compounds of formula 1a are those wherein at least one of the substituents on R₅ is a halogen.

Even more preferred compounds of formula 1a are those wherein both substituents on R₅ are independently halogen.

Especially preferred compounds of formula 1a are those wherein
R₅ is 2,6-dichlorophenyl.

Other preferred compounds of formula Ib are those wherein R₅ is benzyl.

Still other preferred compounds of formula Ib are those wherein
R₂ is benzyloxy or phenethyloxy each of which is optionally substituted with 1, 2, or 3 groups that are independently halogen, hydroxyalkyl, halo(C₁-C₄)alkyl, (C₁-C₄)alkoxy, or alkyl.

Still yet other preferred compounds of formula Ib are those wherein
R₂ is phenyloxy, or —S-phenyl, each of which is optionally substituted with 1, or 2 groups that are independently halogen or alkyl.

More preferred compounds of formula 1b are those wherein
R₂ is benzyloxy, which is optionally substituted with 1, or 2, groups that are independently halogen, chloro(C₁-C₄)alkyl, fluoro(C₁-C₄)alkyl, —CH₂OH, methoxy ethoxy, methyl, ethyl, propyl, or isopropyl.

Even more preferred compounds of formula 1b are those wherein
R₂ is 2,4,6-trisubstitutedbenzyloxy; 2,3,4-trisubstitutedbenzyloxy; 3,4-disubstituted benzyloxy; or 2,4-disubstituted benzyloxy; wherein each is optionally substituted with 1, 2, or 3 groups that are independently halogen, hydroxyalkyl, halo(C₁-C₄)alkyl, (C₁-C₄)alkoxy, or alkyl.

Still yet more preferred compounds of formula 1b are those wherein
R₂ is 2,4,6-trihalobenzyloxy; 2,3,4-trihalobenzyloxy; 3,4-dihalobenzyloxy; or 2,4-dihalobenzyloxy.

Still yet even more preferred compounds of formula 1b are those wherein
R₂ is 2,4,6-trifluorobenzyloxy; 2,3,4-trifluorobenzyloxy; 3,4-difluorobenzyloxy; or 2,4-difluorobenzyloxy.

Other preferred compounds of formula I are those of formula Ic, wherein
R₂ is NR₆R₇, wherein
   R₆ and R₇ are independently at each occurrence selected from H, NH₂, alkyl, hydroxyalkyl, arylalkyl, alkanoyl, cycloalkyl optionally substituted with phenyl, aryl, and heterocycloalkylalkyl,
      wherein the aryl group is optionally substituted with 1, 2, 3, 4, or 5 groups that are independently halogen, alkyl, NH₂, monoalkylamino, dialkylamino, or alkoxy,
      wherein the alkyl portions of the above groups are optionally substituted with 1, 2, or 3 groups that are independently CO₂H, OH, hydroxy (C₁-C₄)alkyl, (C₁-C₄)alkyl, or alkoxycarbonyl, or
   R₆, R₇ and the nitrogen to which they are attached form a piperazine ring which is optionally substituted with 1, 2, or 3 groups that are independently phenyl, phenylalkyl, halogen, or alkyl, wherein the phenyl groups are optionally substituted with 1, 2, or 3 groups that are independently halogen, alkyl, or alkoxy.

Preferred compounds of formula Ic are those wherein
R₆ and R₇ at each occurrence are independently selected from H, NH₂, (C₁-C₆)alkyl, hydroxy (C₁-C₄)alkyl, phenyl(C₁-C₆)alkyl, (C₂-C₆)alkanoyl, (C₃-C₆)cycloalkyl optionally substituted with phenyl, phenyl, and tetrahydrofuryl(C₁-C₆)alkyl,
   wherein the phenyl groups are optionally substituted with 1, 2, 3, 4, or 5 groups that are independently halogen, alkyl, NH₂, or alkoxy,
   wherein the alkyl portions of the above groups are optionally substituted with 1, 2, or 3 groups that are independently CO₂H, OH, hydroxy (C₁-C₄)alkyl, (C₁-C₄)alkyl, or alkoxycarbonyl.

More preferred compounds of formula Ic are those wherein
R₁ is chloro or bromo;
R₃ is H; and
R₅ is benzyl or phenyl, each of which is optionally substituted with 1, 2, or 3 groups that are independently halogen, alkyl or alkoxy.

Even more preferred compounds of formula Ic are those wherein
R₂ is NR₆R₇, wherein
   R₆ is H.

Other preferred compounds of formula 1c are those wherein
R₆, R₇ and the nitrogen to which they are attached form a piperazine ring which is optionally substituted with phenyl or benzyl wherein the phenyl or benzyl groups are optionally substituted with 1, 2, or 3 groups that are independently halogen, alkyl, or alkoxy.

Still yet preferred compounds of formula Ic are those of formula Id, wherein
R₇ is phenyl, benzyl, phenethyl, phenyl(C₃-C₅)alkyl, tetrahydrofuryl(C₁-C₄)alkyl, or cyclopropyl optionally substituted with phenyl,
   wherein the phenyl groups are optionally substituted with 1, 2, 3, 4, or 5 groups that are independently halogen, alkyl, NH₂, or alkoxy,
   wherein the alkyl portions of the above groups are optionally substituted with 1, 2, or 3 groups that are independently CO₂H, OH, hydroxy (C₁-C₄)alkyl, (C₁-C₄)alkyl, or alkoxycarbonyl.

More preferred compounds of formula Id are those wherein
R₁ is bromo or chloro; and
R₇ is benzyl, wherein the phenyl ring is optionally substituted with 1 or 2 groups that are independently halogen or alkyl, and
   the alkyl chain is optionally substituted with 1 or 2 groups that are independently methyl, CO₂H, OH, or (C₁-C₄)alkoxycarbonyl.

Even more preferred compounds of formula Id are those wherein
R₇ is unsubstituted benzyl or 4-halobenzyl.

Still yet more preferred compounds of formula Id are those wherein
R₇ is 4-fluorobenzyl.

Other preferred compounds of formula I are those of formula II 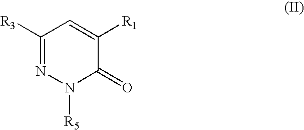
wherein
R₁ is H or C₁-C₆ alkyl; and
R₃ is CO₂H, C(O)NR₆R₇, hydroxyalkyl, aryloxyalkyl, arylalkoxyalkyl, arylalkyl, or —(C₁-C₆)alkylNR₆R₇, wherein
   R₆ and R₇ at each occurrence are independently selected from H, alkyl, arylalkyl, alkanoyl, cycloalkyl optionally substituted with phenyl, aryl, and heterocycloalkylalkyl,
      wherein the aryl group is optionally substituted with 1, 2, 3, 4, or 5 groups that are independently halogen, alkyl, or alkoxy,
      wherein the alkyl portions of the above groups are optionally substituted with 1, 2, or 3 groups that are independently CO2H, alkoxycarbonyl.

Preferred compounds of formula II are those wherein
R₃ is CO₂H, C(O)NR₆R₇, hydroxy(C₁-C₄)alkyl, phenyloxyalkyl, phenylalkoxyalkyl, phenylalkyl, or —(CO₁-C₆)alkylNR₆R₇, wherein
   R₆ and R₇ at each occurrence are independently selected from H, alkyl, phenylalkyl, (C₂-C₆)alkanoyl, phenyl, and heterocycloalkylalkyl,
      wherein the aryl group is optionally substituted with 1, 2, 3, 4, or 5 groups that are independently halogen, alkyl, or alkoxy,
      wherein the alkyl portions of the above groups are optionally substituted with 1, 2, or 3 groups that are independently CO₂H, or alkoxycarbonyl; and
R₅ is phenyl, or phenyl(C₁-C₆)alkyl each of which is optionally substituted with 1, 2, 3, 4, or 5 groups that are independently halogen, alkyl or alkoxy.

More preferred compounds of formula II are those wherein
R₃ is CO₂H, C(O)NHR₇, hydroxy(C₁-C₄)alkyl, phenyloxyalkyl, phenyl(C₁-C₆)alkyl, or —(C₁-C₆)alkylNHR₇, wherein
   R₇ at each occurrence is selected from H, alkyl, phenylalkyl, (C₂-C₆)alkanoyl, phenyl, and tetrahydrofuryl (C₁-C₄)alkyl,
      wherein the phenyl group is optionally substituted with 1, 2, or 3 groups that are independently halogen, alkyl, or alkoxy,
      wherein the alkyl portions of the above groups are optionally substituted with 1, 2, or 3 groups that are independently CO₂H, or (C₁-C₄)alkoxycarbonyl; and
R₅ is phenyl, benzyl, or phenethyl, each of which is optionally substituted with 1, 2, or groups that are independently halogen, alkyl or alkoxy.

Even more preferred compounds of formula II are those wherein
R₃ is C(O)NHR₇, wherein
   R₇ is selected from H, alkyl, benzyl, phenethyl, (C₂-C₆)alkanoyl, phenyl, and tetrahydrofuryl(C₁-C₄)alkyl,
   wherein the phenyl group is optionally substituted with 1, 2, or 3 groups that are independently halogen, alkyl, or alkoxy,
   wherein the alkyl portions of the above groups are optionally substituted with 1, or 2 groups that are independently CO₂H, or (C₁-C₃)alkoxycarbonyl; and
R₅ is phenyl, or benzyl each of which is optionally substituted with 1, or 2 groups that are independently halogen, alkyl or alkoxy.

Still yet more preferred compounds of formula II are those of formula IIa, wherein
R₃ is C(O)NHR₇, wherein
   R₇ is selected from H, alkyl, benzyl, phenethyl, (C₂-C₆)alkanoyl, and phenyl,
      wherein the phenyl group is optionally substituted with 1, 2, or 3 groups that are independently halogen, alkyl, or alkoxy, and
R₅ is 2,6-disubstitutedbenzyl or 2,6-disubstitutedphenyl, each of which is optionally substituted with 1, 2, or 3 groups that are independently halogen, alkyl or alkoxy.

More preferred compounds of formula IIa are those wherein at least one of the substituents on R₅ is a halogen.

Even more preferred compounds of formula IIa are those wherein both substituents on R₅ are independently halogen.

Still more preferred compounds of formula IIa are those wherein
R₅ is 2,6-dichlorophenyl.

Also preferred are compounds of formula II wherein
R₅ is benzyl.

Other more preferred compounds of formula II are those of formula IIb, wherein
R₃ is —(C₁-C₆)alkylNR₆R₇, phenyl(C₁-C₆)alkyl, or phenylalkoxyalkyl, wherein
   R₆ and R₇ at each occurrence are independently selected from H, alkyl, benzyl, phenethyl, (C₂-C₆)alkanoyl, phenyl, and tetrahydrofuryl(C₁-C₄)alkyl,
      wherein the phenyl group is optionally substituted with 1, 2, or 3 groups that are independently halogen, alkyl, or alkoxy,
      wherein the alkyl portions of the above groups are optionally substituted with 1, or 2 groups that are independently CO₂H, or (C₁-C₃)alkoxycarbonyl; and
R₅ is phenyl, benzyl, or phenethyl, each of which is optionally substituted with 1, or 2 groups that are independently halogen, alkyl or alkoxy.

More preferred compounds of formula IIb are those wherein
R₅ is 2,6-disubstitutedbenzyl, benzyl, phenyl, or 2,6-disubstitutedphenyl, each of which is optionally substituted with 1, 2, or 3 groups that are independently halogen, alkyl, or alkoxy.

Even more preferred compounds of formula IIb are those wherein
R₅ is benzyl, or 2,6-disubstitutedphenyl, each of which is optionally substituted with 1, or 2 groups that are independently halogen, alkyl, or alkoxy.

Still more preferred compounds of formula IIb are those wherein
R₃ is —(C₁-C₆)alkylNR₆R₇;
   R₆ and R₇ at each occurrence are independently selected from H, alkyl, benzyl, phenethyl, and (C₂-C₆)alkanoyl, and phenyl,
      wherein the phenyl group is optionally substituted with 1, or 2 groups that are independently halogen, alkyl, or alkoxy,

Other even more preferred compounds of formula IIb are those wherein
R₅ is benzyl, or 2,6-dichlorophenyl and
R₆ is H.

Still other even more preferred compounds of formula IIb are those wherein
R₃ is phenyl(C₁-C₆)alkyl.

Still yet even more preferred compounds of formula IIb are those wherein,
R₅ is benzyl, or 2,6-dichlorophenyl.

Other even more preferred compounds of formula IIb are those of formula IIc wherein
R₃ is phenyl(C₁-C₄)alkoxy(C₁-C₄)alkyl, such as —CH₂OCH₂phenyl or —CH₂OCH₂CH₂phenyl.

More preferred compounds of formula IIc are those wherein
R₅ is benzyl, or 2,6-dichlorophenyl.

Other preferred compounds of formula I are those of the formula 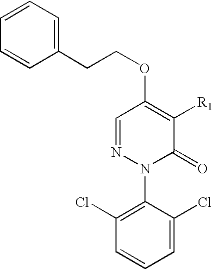
wherein
R₁ is H, halogen, (C₁-C₆)alkyl, phenyl, (C₁-C₆)alkoxy, or phenyloxy, each of which is optionally substituted with 1, 2, 3 or 4 groups that are independently halogen, methyl, or methoxy.

Still other preferred compounds of formula I are those of the formula: 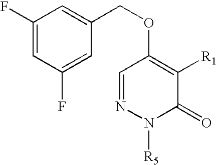
wherein
R₁ is halogen;
   R₅ is H, phenyl, pyridyl(C₁-C₆)alkyl, NH₂alkyl, (C₁-C₆)alkyl-NH—(C₁-C₆)alkyl, di(C₁-C₆)alkylamino(C₁-C₆)alkyl, hydroxy(C₁-C₆)alkyl, thiazolyl, or thiazolylalkyl, each of which is optionally substituted with 1, 2, or 3 groups that are independently halogen, methoxy, or methyl.

Preferred embodiments of the invention include:

### EMBODIMENT 2

Compounds of the Formula I, having the formula: 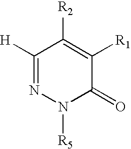
or a pharmaceutically acceptable salt thereof, wherein
R₁ is H, halogen, alkyl, carboxaldehyde, hydroxyalkyl, dihydroxyalkyl, arylalkoxy, arylalkyl, alkenyl, alkynyl, arylalkynyl, CN, alkanoyl, alkoxy, alkoxyalkyl, haloalkyl, carboxyl, or arylalkanoyl,
   wherein the aryl portion of arylalkoxy, arylalkyl, and arylalkanoyl is unsubstituted or substituted with 1, 2, 3, 4, or 5 groups that are independently halogen, C₁-C₄ alkyl, C₁-C₄ alkoxy, nitro, CN, haloalkyl, haloalkoxy or CO₂R;
   wherein the alkyl portion of the alkyl, hydroxyalkyl, dihydroxyalkyl, arylalkoxy, arylalkyl, alkanoyl, alkoxy, alkoxyalkyl and arylalkanoyl groups is unsubstituted or substituted with 1, 2, or 3 groups that are independently halogen, C₁-C₄ alkoxy, C₁-C₄ alkoxycarbonyl, or cyclopropyl;
R₂ is H, OH, halogen, —OSO₂—(C₁-C₆)alkyl, —OSO₂-aryl, arylthio, arylalkylthio, arylamino (C₁-C₆)alkyl, arylalkylamino, arylalkoxy, aryloxy, arylthioalkoxy, arylalkynyl, alkoxy, phenyloxy(C₁-C₆)alkyl, —OC(O)NH(CH₂)ₙaryl, —OC(O)N(alkyl)(CH₂)ₙaryl, alkyl, alkynyl, alkoxyalkoxy, dialkylamino, heteroaryl, heterocycloalkyl, aryloxyalkyl, or CO₂R, wherein
   each of the above is unsubstituted or substituted with 1, 2, 3, 4, or 5 groups that are independently halogen, —NR₆R₇, haloalkyl, haloalkoxy, alkyl, heteroaryl, heteroarylalkyl, —(C₁-C₆alkyl)-C(O)—NR₆R₇, R₆R₇N—(C₁-C₆ alkyl)-, —C(O)NR₆R₇, —(C₁-C₄ alkyl)-NRC(O)NR₁₆R₁₇, CN, hydroxyalkyl, dihydroxyalkyl, —OC(O)NR₆R₇, or —(C₁-C₆)alkyl-N(R)—CO₂R₃₀, wherein
      R₁₆ and R₁₇ are independently H or C₁-C₆ alkyl; or
      R₁₆, R₁₇ and the nitrogen to which they are attached form a morpholinyl ring;
      R₆ and R₇ are independently at each occurrence H, alkyl, hydroxyalkyl, dihydroxyalkyl, alkoxy, alkoxyalkyl, alkanoyl, arylalkyl, arylalkoxy, arylalkoxycarbonyl, or arylalkanoyl, wherein each of the above is unsubstituted or substituted with 1, 2, or 3 groups that are independently, halogen, alkoxy, alkyl, OH, SH, carboxaldehyde, haloalkyl, or haloalkoxy; or
      R₆, R₇, and the nitrogen to which they are attached form a morpholinyl, thiomorpholinyl, thiomorpholinyl S-oxide, thiomorpholinyl S,S-dioxide, piperidinyl, pyrrolidinyl, or piperazinyl ring which is optionally substituted with 1 or 2 groups that are independently C₁-C₄ alkyl, alkoxycarbonyl, hydroxyl, hydroxyalkyl, dihydroxyalkyl, or halogen;
   n is 0, 1, 2, 3, 4, 5 or 6;
   R at each occurrence is independently H or C₁-C₆ alkyl optionally substituted with 1 or 2 groups that are independently OH, SH, halogen, amino, monoalkylamino, dialkylamino or C₃-C₆ cycloalkyl;
   R₃₀ is C₁-C₆ alkyl optionally substituted with 1 or 2 groups that are independently OH, SH, halogen, amino, monoalkylamino, dialkylamino or C₃-C₆ cycloalkyl; and
R₅ is H, arylalkyl, alkyl optionally substituted with 1, 2, or 3 groups that are independently arylalkoxycarbonyl, —NR₈R₉, halogen, —C(O)NR₈R₉, alkoxycarbonyl, or alkanoyl, alkoxyalkyl optionally substituted with one trimethylsilyl group, alkoxycarbonyl, amino, hydroxyalkyl, dihydroxyalkyl, alkenyl optionally substituted with alkoxycarbonyl, alkynyl, —SO₂-alkyl, aryl, alkoxy optionally substituted with one trimethylsilyl group, heterocycloalkylalkyl, heteroarylalkyl, heterocycloalkyl, or heteroaryl, wherein
   each of the above is unsubstituted or substituted with 1, 2, 3, 4, or 5 groups that are independently alkyl, halogen, alkoxy, arylalkoxy, hydroxyalkyl, dihydroxyalkyl, thioalkoxy, —SO₂alkyl, alkoxycarbonyl, arylalkoxycarbonyl, CO₂R, CN, OH, amidinooxime, NR₈R₉, R₆R₇N—(C₁-C₆ alkyl)-, —C(O)NR₆R₇, amidino, hydroxyalkyl, dihydroxyalkyl, carboxaldehyde, —NR₆R₇, haloalkyl, —(C₁-C₄ alkyl)-C(O)NR₆R₇, —(C₁-C₄ alkyl)-CO₂R, —(C₁-C₄ alkyl)-C₁-C₆ alkoxycarbonyl, —(C₁-C₄ alkyl)-CN, —(C₁-C₄ alkyl)-NR₁₅C(O)R₁₈, —O—CH₂—O—, —O—CH₂CH₂—O—, phenyl or haloalkoxy;
      R₈ is hydrogen, alkyl, alkanoyl, arylalkyl and arylalkanoyl;
      R₉ is alkyl, alkanoyl, arylalkyl, heteroaryl, aminoalkyl, monoalkylaminoalkyl, dialkylaminoalkyl, and arylalkanoyl.

### EMBODIMENT 3

Compounds according to embodiment 2 wherein
R₁ is H, halogen, alkyl optionally substituted with C₁-C₄ alkoxycarbonyl, carboxaldehyde, hydroxyalkyl, dihydroxyalkyl, phenyl(C₁-C₆)alkoxy, phenyl (C₁-C₆)alkyl, CN, alkanoyl, alkoxy, C₂-C₄ alkynyl, C₂-C₆ alkenyl optionally substituted with C₁-C₄ alkoxycarbonyl, alkoxyalkyl, haloalkyl, or phenyl (C₁-C₆)alkanoyl,
   wherein the phenyl groups are unsubstituted or substituted with 1, 2, 3, 4, or 5 groups that are independently halogen, C₁-C₄ alkyl, C₁-C₄ alkoxy, nitro, CN, CF₃, OCF₃ or CO₂R;
   wherein the alkyl groups are unsubstituted or substituted with 1, 2, or 3 groups that are independently halogen, methoxy, or ethoxy;
R₂ is OH, phenyl(C₁-C₆)alkoxy, phenyloxy, phenyloxy(C₁-C₆)alkyl, phenylthio, phenylalkylthio, phenylamino (C₁-C₆)alkyl, phenylalkylamino, phenyl(C₁-C₄)thioalkoxy, C₁-C₈ alkoxy, alkoxyalkoxy, —O—SO₂phenyl, alkynyl, phenyl (C₂-C₄) alkynyl, alkyl, —OC(O)NH(CH₂)ₙphenyl, —OC(O)N(alkyl)(CH₂)ₙphenyl, dialkylamino, pyridyl, pyrimidyl, pyridazyl, pyrazolyl, imidazolyl, pyrrolyl, tetrahydroquinolinyl, tetrahydroisoquinolinyl, tetrazolyl, pyrazinyl, benzimidazolyl, triazinyl, tetrahydrofuryl, piperidinyl, hexahydropyrimidinyl, thiazolyl, thienyl, or CO₂R, wherein
   n is 0, 1, 2, 3, 4, 5 or 6;
   each of the above is unsubstituted or substituted with 1, 2, 3, 4, or 5 groups that are independently halogen, NR₆R₇, haloalkyl, haloalkoxy, hydroxyalkyl, dihydroxyalkyl, alkyl, phenyl, pyridyl, piperidinyl, piperazinyl, —(C₁-C₆alkyl)-C(O)—NR₆R₇, —(C₁-C₆)alkyl-N(R)—CO₂R₃₀, R₆R₇N—(C₁-C₆ alkyl)-, —C(O)NR₆R₇, —(C₁-C₄ alkyl)-NRC(O)NR₁₆R₁₇, or —OC(O)NR₆R₇, wherein
      R₆ and R₇ are independently at each occurrence H, alkyl, (C₁-C₄)hydroxyalkyl, (C₁-C₄) dihydroxyalkyl, (C₁-C₄)alkoxy, (C₁-C₄)alkoxy (C₁-C₄)alkyl, (C₁-C₄)alkanoyl, phenyl (C₁-C₄) alkyl, phenyl (C₁-C₄)alkoxy, phenyl (C₁-C₄) alkoxycarbonyl, or phenyl (C₁-C₄)alkanoyl, wherein each of the above is unsubstituted or substituted with 1, 2, or 3 groups that are independently, halogen, OH, SH, C₃-C₆ cycloalkyl, (C₁-C₄)alkoxy, (C₁-C₄)alkyl, CF₃, carboxaldehyde, NH₂, NH(C₁-C₆)alkyl, N(C₁-C₆)alkyl (C₁-C₆)alkyl, OCF₃; or
      R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, hydroxy, hydroxy C₁-C₄ alkyl, C₁-C₄ dihydroxyalkyl, C₁-C₄ alkoxycarbonyl, or halogen; and
R₅ is phenyl(C₁-C₆)alkyl, (C₁-C₆)alkyl optionally substituted with 1, 2, 3, 4, or 5 groups that are independently phenyl C₁-C₄ alkoxycarbonyl, —NR₈R₉, halogen, —C(O)NR₈R₉, alkoxycarbonyl, or alkanoyl, phenyl, alkoxy, C₂-C₆ alkynyl, C₂-C₆ alkenyl optionally substituted with alkoxycarbonyl, indolyl, quinolinyl, isoquinolinyl, isoindolyl, dihydroindolyl, dihydroisoindolyl, indolon-2-yl, indazolyl, benzimidazolyl, pyridyl, imidazolidine dione, pyridyl (C₁-C₆)alkyl, pyridazinyl (C₁-C₆)alkyl, pyrimidinyl (C₁-C₆)alkyl, pyrazinyl (C₁-C₆)alkyl, tetrahydrofuryl(C₁-C₆)alkyl, naphthyl(C₁-C₆)alkyl, morpholinyl (C₁-C₆)alkyl, tetrahydrofuryl(C₁-C₆)alkyl, thienyl (C₁-C₆)alkyl, piperazinyl (C₁-C₆)alkyl, indolyl (C₁-C₆)alkyl, quinolinyl(C₁-C₆)alkyl, isoquinolinyl(C₁-C₆)alkyl, isoindolyl(C₁-C₆)alkyl, dihydroindolyl(C₁-C₆) alkyl, dihydroisoindolyl(C₁-C₆)alkyl, indoon-2-yl(C₁-C₆) alkyl, indolon-2-yl(C₁-C₆)alkyl, or morpholinyl C₁-C₆ alkyl, wherein
   each of the above is unsubstituted or substituted with 1, 2, 3, 4, or 5 groups that are independently C₁-C₆ alkyl, halogen, C₁-C₆ alkoxy, phenyl C₁-C₆ alkoxy, C₁-C₆ thioalkoxy, C₁-C₆ alkoxycarbonyl, CO₂R, CN, —SO₂ (C₁-C₆)alkyl, amidinooxime, NR₈R₉, —NR₆R₇, NR₆R₇ C₁-C₆ alkyl, —C(O)NR₆R₇, amidino, —(C₁-C₆alkyl)-C(O)—NR₆R₇, C₁-C₄ haloalkyl, hydroxy C₁-C₆ alkyl, C₁-C₆ dihydroxyalkyl, or C₁-C₄ haloalkoxy; wherein
      R₈ is hydrogen, C₁-C₆ alkyl, C₁-C₆ alkanoyl, phenyl C₁-C₆ alkyl and phenyl C₁-C₆ alkanoyl; and
      R₉ is aminoalkyl, mono C₁-C₆ alkylamino C₁-C₆ alkyl, di C₁-C₆ alkylamino C₁-C₆ alkyl, C₁-C₆ alkyl, C₁-C₆ alkanoyl, phenyl C₁-C₆ alkyl, indazolyl, and phenyl C₁-C₆ alkanoyl.

### EMBODIMENT 4

Compounds according to embodiment 3, wherein
R₁ is H, halogen, C₁-₄ alkyl optionally substituted with C₁-₄ alkoxycarbonyl, C₂-C₄ alkenyl optionally substituted with C₁-₄ alkoxycarbonyl, C₂-C₄ alkynyl, or carboxaldehyde;
R₂ is benzyloxy, OH, phenyloxy, phenyloxy(C₁-C₆)alkyl, phenyl (C₁-₄)thioalkoxy, or pyridyl; wherein each of the above is optionally substituted with 1, 2, 3, 4, or 5 groups that are independently halogen, —(C₁-C₆)alkyl-N(R)-CO₂R₃₀, —(C₁-C₆alkyl)-C(O)—NR₆R₇, NR₆R₇, (C₁-C₄)haloalkyl, —C(O)NR₆R₇, —(C₁-C₄ alkyl)-NRC(O)NR₁₆R_{17,} (C₁-C₄)haloalkoxy, hydroxyalkyl, C₁-C₆ dihydroxyalkyl, (C₁-C₆)alkyl, pyridyl, or R₆R₇N—(C₁-C₆ alkyl)-.

### EMBODIMENT 5

Compounds according to embodiment 4, wherein
R₅ is indolyl, pyridyl, pyridazinyl, pyrimidinyl, indazolyl, quinolinyl, isoquinolinyl, isoindolyl, dihydroindolyl, dihydroisoindolyl, indolon-2-yl, pyridazinyl, pyrimidinyl, or pyrazinyl, each of which is unsubstituted or substituted with 1, 2, 3, 4 or 5 groups that are independently C₁-C₄ alkyl, halogen, CF₃, OCF₃, —CO₂CH₃, C₁-C₄ hydroxyalkyl, dihydroxyalkyl, C₁-C₄ alkoxy, —CO₂(C₁-C₅ alkyl), benzyloxy, —NR₆R₇, —NR₈R₉, NR₆R₇—(C₁-C₄ alkyl), —C(O)NR₆R₇, or amidinooxime; wherein
   R₆ and R₇ are independently at each occurrence H, C₁-C₄ alkyl, C₁-C₄ hydroxyalkyl, C₁-C₄ dihydroxyalkyl, C₁-C₄ alkoxy, C₁-C₄ alkoxy C₁-C₄ alkyl, C₁-C₄ alkanoyl, phenyl C₁-₄ alkyl, phenyl C₁-C₄ alkoxy, or phenyl C₁-C₄ alkanoyl, wherein each is unsubstituted or substituted with 1, 2, or 3 groups that are independently, halogen, OH, SH, C₃-C₆ cycloalkyl, C₁-C₄ alkoxy, C₁-C₄ alkyl, OH, CF₃, or OCF₃; or
   R₆, R₇, and the nitrogen to which they are attached form a morpholinyl, thiomorpholinyl, pyrrolidinyl, or piperazinyl ring which is optionally substituted with 1 or 2 groups that are independently C₁-C₄ alkyl, hydroxy, hydroxy C₁-C₄ alkyl, C₁-C₄ dihydroxyalkyl, or halogen.

### EMBODIMENT 6

Compounds according to embodiment 5, wherein
R₅ is indolyl, pyridyl, pyrimidinyl, indazolyl, dihydroindolyl, dihydroisoindolyl, indolon-2-yl, or pyrazinyl, each of which is unsubstituted or substituted with 1, 2, 3, or 4 groups that are independently C₁-C₄ alkyl, halogen, CF₃, OCF₃, —CO₂CH₃, C₁-C₄ hydroxyalkyl, C₁-C₄ dihydroxyalkyl, C₁-C₄ alkoxy, —CO₂(C₁-C₅ alkyl), benzyloxy, —C(O)NR₆R₇, —NR₈R₉, —NR₆R₇, NR₆R₇—(C₁-C₄ alkyl)-, and amidinooxime.

### EMBODIMENT 7

Compounds according to embodiment 6, wherein
R₅ is indolyl, pyridyl, pyrimidinyl, dihydroindolyl, or pyrazinyl, each of which is unsubstituted or substituted with 1, 2, 3, or 4 groups that are independently C₁-C₄ alkyl, halogen, CF₃, OCF₃, —CO₂CH₃, C₁-C₄ hydroxyalkyl, C₁-C₄ dihydroxyalkyl, C₁-C₄ alkoxy, —CO₂(C₁-C₅ alkyl), benzyloxy, —C(O)NR₆R₇, NR₈R₉, —NR₆R₇, NR₆R₇—(C₁-C₄ alkyl)-, or amidinooxime; wherein
   R₆ and R₇ are independently at each occurrence H, C₁-C₄ alkyl, C₁-C₄ hydroxyalkyl, C₁-C₄ dihydroxyalkyl, C₁-C₄ alkoxy, C₁-C₄ alkanoyl, C₁-C₄ alkoxy C₁-C₄ alkyl, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃.

### EMBODIMENT 8

Compounds according to embodiment 7, wherein
R₅ is indolyl, pyridyl, pyrimidinyl, dihydroindolyl, or pyrazinyl, each of which is unsubstituted or substituted with 1, 2, or 3 groups that are independently C₁-C₄ alkyl, halogen, CF₃, OCF₃, C₁-C₄ hydroxyalkyl, C₁-C₄ dihydroxyalkyl, C₁-C₄ alkoxy, —C(O)NR₆R₇, NR₈R₉, —NR₆R₇, or NR₆R₇—(C₁-C₄ alkyl)-; wherein
   R₆ and R₇ are independently at each occurrence H, C₁-C₄ alkyl, C₁-C₄ hydroxyalkyl, C₁-C₄ dihydroxyalkyl, C₁-C₄ alkanoyl, or C₁-C₄ alkoxy, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃.

### EMBODIMENT 9

Compounds according to embodiment 4, wherein
R₅ is phenyl(C₁-C₆)alkyl, or (C₁-C₆)alkyl, wherein
   each of the above is unsubstituted or substituted with 1, 2, 3, 4, or 5 groups that are independently alkyl, halogen, alkoxy, benzyloxy, hydroxyalkyl, dihydroxyalkyl, thioalkoxy, —CO₂(C₁-C₅ alkyl), CO₂R, CN, amidinooxime, —NR₈R₉, —NR₆R₇, R₆R₇N—(C₁-C₆ alkyl)-, —C(O)NR₆R_{7,} amidino, CF₃, or OCF₃;
   R₈ is hydrogen, C₁-C₆ alkyl, C₁-C₆ alkanoyl, phenyl C₁-C₆ alkyl and phenyl C₁-C₆ alkanoyl; and
   R₉ is aminoalkyl, mono C₁-C₆ alkylamino C₁-C₆ alkyl, di C₁-C₆ alkylamino C₁-C₆ alkyl, C₁-C₆ alkyl, C₁-C₆ alkanoyl, phenyl C₁-C₄ alkyl, indazolyl, and phenyl C₁-₄ alkanoyl.

### EMBODIMENT 10

Compounds according to embodiment 4, wherein
R₅ is phenyl(C₁-C₆)alkyl, which is unsubstituted or substituted with 1, 2, 3, 4, or 5 groups that are independently alkyl, halogen, alkoxy, benzyloxy, thioalkoxy, —CO₂(C₁-C₅ alkyl), CO₂R, CN, amidinooxime, —NR₈R₉, —NR₆R₇, R₆R₇N—(C₁-C₆ alkyl)-, —C(O)NR₆R₇, amidino, CF₃, or OCF₃; wherein
   R₆ and R₇ are independently at each occurrence H, C₁-C₄ alkyl, C₁-C₄ hydroxyalkyl, C₁-C₄ dihydroxyalkyl, C₁-C₄ alkoxy, C₁-C₄ alkoxy C₁-C₄ alkyl, C₁-C₄ alkanoyl, phenyl C₁-C₄ alkyl, phenyl C₁-C₄ alkoxy, or phenyl C₁-C₄ alkanoyl, wherein each is unsubstituted or substituted with 1, 2, or 3 groups that are independently, halogen, OH, SH, C₃-C₆ cycloalkyl, C₁-C₄ alkoxy, C₁-C₄ alkyl, CF₃, or OCF₃; or
   R₆, R₇, and the nitrogen to which they are attached form a morpholinyl, thiomorpholinyl, or piperazinyl ring which is optionally substituted with 1 or 2 groups that are independently C₁-C₄ alkyl, hydroxy, hydroxy C₁-C₄ alkyl, C₁-C₄ dihydroxyalkyl, or halogen;
   R₈ is hydrogen, C₁-C₆ alkyl, C₁-C₆ alkanoyl, phenyl C₁-C₆ alkyl and phenyl C₁-C₆ alkanoyl; and
   R₉ is aminoalkyl, mono C₁-C₆ alkylamino C₁-C₆ alkyl, di C₁-C₆ alkylamino C₁-C₆ alkyl, C₁-C₆ alkyl, C₁-C₆ alkanoyl, phenyl C₁-C₄ alkyl, indazolyl, and phenyl C₁-C₄ alkanoyl.

### EMBODIMENT 11

Compounds according to embodiment 10, wherein
R₅ is benzyl or phenethyl, wherein each is optionally substituted with 1, 2, 3, 4, or 5 groups that are independently C₁-C₆ alkyl, —NR₆R₇, —C(O)NR₆R₇, —(C₁-C₄ alkyl)-C(O)NR₆R₇, —NR₈R₉, halogen, C₁-C₆ alkoxy, CO₂R, —(C₁-C₄ alkyl)-CO₂R, C₁-C₆ thioalkoxy, amidinooxime, C₁-C₆ alkoxycarbonyl, —(C₁-C₄ alkyl)-C₁-C₆ alkoxycarbonyl, C₁-C₆ hydroxyalkyl, C₁-C₆ dihydroxyalkyl, —(C₁-C₄ alkyl)-CN, CN, phenyl C₁-C₆ alkoxy, OH, C₁-C₄ haloalkyl, C₁-C₄ haloalkoxy, R₆R₇N—(C₁-C₆ alkyl)-, —(C₁-C₄ alkyl)-NR₁₅C(O)R₁₈, amidinooxime, —SO₂(C₁-C₆ alkyl), —O—CH₂—O—, —O—CH₂CH₂—O—, phenyl C₁-C₄ alkoxy, or phenyl; wherein
   R₆ and R₇ are independently at each occurrence H, C₁-C₄ alkyl, C₁-C₄ hydroxyalkyl, C₁-C₄ dihydroxyalkyl, C₁-C₄ alkanoyl, or C₁-C₄ alkoxy, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃.

### EMBODIMENT 12

Compounds according to embodiment 11, wherein
R₅ is benzyl or phenethyl, each of which is unsubstituted or substituted with 1, 2, 3, 4, or 5 groups that are independently CN, halogen, C₁-C₄ alkoxy, CF₃, OCF₃, C₁-C₄ alkyl, —NR₈R₉, —NR₆R₇, R₆R₇N-(C₁-C₆ alkyl)-, or —C(O)NR₆R₇, wherein
   R₆ and R₇ are independently at each occurrence H, C₁-C₄ alkyl, C₁-C₄ hydroxyalkyl, C₁-C₄ dihydroxyalkyl, C₁-C₄ alkanoyl, or C₁-C₄ alkoxy, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃.

### EMBODIMENT 13. Compounds according to embodiment 4, wherein

the R₅ group is of the formula: 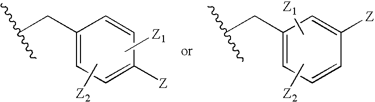
   wherein
Z₁ and Z₂ are independently H, halogen, C₁-C₄ alkyl, or CO₂R; and
Z is —C(O)NR₆R₇, —(C₁-₄ alkyl)-NR₁₅C(O)R₁₈, —NR₆R₇, R₆R₇N—(C₁-C₆ alkyl)-, —NR₈R₉, C₁-C₆ hydroxyalkyl, C₁-C₆ dihydroxyalkyl, C₁-C₆ alkyl, CO₂R, or halogen; wherein
   R₆ and R₇ at each occurrence are independently H, OH, C₁-C₆ alkyl, amino C₁-C₄ alkyl, NH(C₁-C₆ alkyl)alkyl, N(C₁-C₆ alkyl)(C₁-C₆ alkyl) C₁-C₆ alkyl, C₁-C₆ hydroxyalkyl, C₁-C₆ dihydroxyalkyl, C₁-C₆ alkoxy C₁-C₆ alkyl, or —SO₂(C₁-C₆ alkyl) each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃; or
   R₆, R₇, and the nitrogen to which they are attached form a piperidinyl, pyrrolidinyl, piperazinyl, or a morpholinyl, thiomorpholinyl, ring optionally substituted with 1 or 2 groups that are independently alkyl, hydroxy, hydroxy C₁-C₄ alkyl, C₁-C₄ dihydroxyalkyl, or halogen; and
   R₁₈ is C₁-C₆ alkyl optionally substituted with —O—(C₂-C₆ alkanoyl, C₁-C₆ hydroxyalkyl, C₁-C₄ dihydroxyalkyl, C₁-C₆ alkoxy, C₁-C₆ alkoxy C₁-C₆ alkyl; amino C₁-C₆ alkyl, mono or dialkylamino C₁-C₆ alkyl.

### EMBODIMENT 14

Compounds according to embodiment 1, wherein
R₅ is pyrazolyl(C₁-C₆ alkyl), imidazolyl(C₁-C₆ alkyl), furanyl(C₁-C₆ alkyl), thienyl(C₁-C₆ alkyl), piperidinyl (C₁-C₆)alkyl, pyrrolidinyl (C₁-C₆)alkyl, imidazolidinyl(C₁-C₆)alkyl, piperazinyl (C₁-C₆)alkyl, pyridyl (C₁-C₆)alkyl, pyrimidyl(C₁-C₆)alkyl, pyridazyl(C₁-C₆)alkyl, pyrazinyl(C₁-C₆)alkyl, isoquinolinyl(C₁-C₆)alkyl, tetrahydroisoquinolinyl (C₁-C₆)alkyl, indolyl(C₁-C₆)alkyl, 1H-indazolyl (C₁-C₆)alkyl, dihydroindolyl(C₁-C₆ alkyl), dihydroindolon-2-yl(C₁-C₆ alkyl), indolinyl(C₁-C₆ alkyl), dihydroisoindolyl(C₁-C₆ alkyl), dihydrobenzimdazolyl(C₁-C₆ alkyl), or dihydrobenzoimidazolonyl(C₁-C₆ alkyl), wherein
   each of the above is unsubstituted or substituted with 1, 2, 3, 4, or 5 groups that are independently (C₁-C₆)alkyl, halogen, (C₁-C₆)alkoxy, (C₁-C₆)hydroxyalkyl, C₁-C₆ dihydroxyalkyl, phenyl(C₁-C₆)alkoxy, (C₁-C₆)thioalkoxy, (C₁-C₆)alkoxycarbonyl, phenyl(C₁-C₆)alkoxycarbonyl, OH, CO₂R, CN, amidinooxime, —NR₈R₉, —NR₆R₇, R₆R₇N—(C₁-C₆ alkyl)-, —C(O)NR₆R₇, —(C₁-C₄ alkyl)-C(O)NR₆R₇, amidino, piperazinyl, morpholinyl, —SO₂ (C₁-C₆)alkyl, —SO₂NH₂, —SO₂NH(C₁-C₆)alkyl, —SO₂N(C₁-C₆)alkyl (C₁-C₆)alkyl, (C₁-C₄)haloalkyl, -(C₁-C₄ alkyl)-NR₁₅C(O)NR₁₆R₁₇, —(C₁-C₄ alkyl)-NR₁₅C(O)R₁₈, —O—CH₂—O, —O—CH₂CH₂—O—, or (C₁-C₄)haloalkoxy; wherein
      R₆ and R₇ are independently at each occurrence H, (C₁-C₆)alkyl, (C₁-C₆)alkoxy, (C₁-C₆)alkoxy(C₁-C₆)alkyl, (C₁-C₆)alkoxycarbonyl, (C₁-C₆)hydroxyalkyl, C₁-C₆ dihydroxyalkyl, —(C₁-C₄)alkyl-CO₂—(C₁-C₆)alkyl, (C₁-C₆)alkanoyl, phenyl(C₁-C₆)alkyl, phenyl(C₁-C₆)alkoxy, or phenyl(C₁-C₆)alkanoyl, wherein each of the above is unsubstituted or substituted with 1, 2, or 3 groups that are independently, halogen, (C₁-C₄)alkoxy, OH, SH, C₃-C₆ cycloalkyl, NH₂, NH(C₁-C₆ alkyl), N(C₁-C₆ alkyl)(C₁-C₆ alkyl), (C₁-C₄)alkyl, CF₃ or OCF₃; or
      R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, hydroxy, hydroxy C₁-C₄ alkyl, C₁-C₄ dihydroxyalkyl, or halogen; and
      R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl,
provided that R₆ and R₇ are not simultaneously OH;
provided that R₆ and R₇ are not simultaneously —SO₂(C₁-C₆ alkyl).

### EMBODIMENT 15

Compounds according to embodiment 14, wherein
R₅ is thienyl(C₁-C₆ alkyl), pyrimidyl(C₁-C₆)alkyl, pyrazolyl(C₁-C₆ alkyl), indolyl(C₁-C₆ alkyl), dihydroindolyl(C₁-C₆ alkyl), dihydroisoindolyl(C₁-C₆ alkyl), dihydroindolon-2-yl(C₁-C₆ alkyl), pyridinyl(C₁-C₆ alkyl), piperazinyl(C₁-C₆ alkyl), or pyrazinyl(C₁-C₆ alkyl) each of which is optionally substituted with 1, 2, or 3 groups that are independently C₁-C₄ alkyl, C₁-C₄ hydroxyalkyl, C₁-C₄ dihydroxyalkyl, halogen, —C(O)NR₆R₇, —(C₁-C₄ alkyl)-C(O)NR₆R₇, C₁-C₆ alkoxycarbonyl, —NR₆R₇, R₆R₇N-(C₁-C₆ alkyl)-, haloalkyl, C₁-C₆ alkanoyl,
   R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy;
   or
   R₆, R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen.

### EMBODIMENT 16

Compounds according to embodiment 15, wherein
R₅ is of the formula: 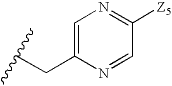
   wherein
Z₅ is C₁-C₄ alkyl, C₁-C₄ hydroxyalkyl, C₁-C₄ dihydroxyalkyl, halogen, —C(O)NR6R₇, —(C₁-C₄ alkyl)-C(O)NR₆R₇, C₁-C₆ alkoxycarbonyl, R₆R₇N—(C₁-C₆ alkyl)-, —NR₆R₇, CF₃, or C₁-C₆ alkanoyl, wherein
   R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy;
   or
   R₆, R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen.

### EMBODIMENT 17

Compounds according to embodiment 15, wherein
R₅ is of the formula: 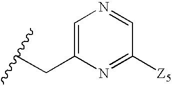
   wherein
Z₅ is C₁-C₄ alkyl, C₁-C₄ hydroxyalkyl, C₁-C₄ dihydroxyalkyl, halogen, —C(O)NR₆R₇, —(C₁-C₄ alkyl)-C(O)NR₆R₇, C₁-C₆ alkoxycarbonyl, R₆R₇N—(C₁-C₆ alkyl)-, —NR₆R₇, CF₃, or C₁-C₆ alkanoyl, wherein
   R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy;
   or
   R_{6,} R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen.

### EMBODIMENT 18

Compounds according to either embodiment 16 or 17, wherein
Z₅ is C₁-C₄ alkyl, C₁-C₄ hydroxyalkyl, C₁-C₄ dihydroxyalkyl, halogen, C₁-C₆ alkoxycarbonyl, CF₃, or C₁-C₆ alkanoyl.

### EMBODIMENT 19

Compounds according to either embodiment 16 or 17, wherein
Z₅ is —C(O)NR₆R₇, —(C₁-C₄ alkyl)-C(O)NR₆R₇, R₆R₇N—(C₁-C₆ alkyl)-, or —NR₆R₇, CF₃, or C₁-C₄ alkanoyl, wherein
   R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy;
   or
   R₆, R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen.

### EMBODIMENT 20

Compounds according to embodiment 19, wherein
Z₅ is —C(O)NR₆R₇, —(C₁-C₄ alkyl)-C(O)NR₆R₇, R₆R₇N—(C₁-C₆ alkyl)-, or —NR₆R₇, wherein
R₆ and R₇ at each occurrence are independently H, C₁-C₆ alkyl optionally substituted with 1, 2, or 3 groups that are independently C₁-C₄ alkoxycarbonyl, halogen, cyclopropyl, OH, SH, or C₁-C₄ alkoxy.

### EMBODIMENT 21

Compounds according to embodiment 15, wherein
R₅ is of the formula: 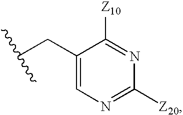
   wherein
Z₁₀ is H or methyl; and
Z₂₀ is hydroxy(C₁-C₄)alkyl, C₁-C₄ dihydroxyalkyl, OH, halogen, haloalkyl, (C₁-C₄)alkyl, OCF₃, —NR₆R₇, R₆R₇N—(C₁-C₆ alkyl)-, or —C(O)NR₆R₇, wherein
R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy.

### EMBODIMENT 22

Compounds according to embodiment 15, wherein
R₅ is of the formula: 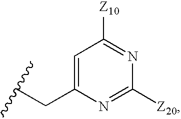
   wherein
Z₁₀ is H or methyl; and
Z₂₀ is hydroxy(C₁-C₄)alkyl, C₁-C₄ dihydroxyalkyl, OH, halogen, CF₃, (C₁-C₄)alkyl, OCF₃, —NR₆R₇, R₆R₇N—(C₁-C₆ alkyl)-, or —C(O)NR₆R₇, wherein
R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy.

### EMBODIMENT 23

Compounds according to embodiment 4, wherein
R₅ is phenyl, which is optionally substituted with 1, 2, 3, 4, or 5 groups that are independently C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, dihydroxyalkyl, halogen, C₁-C₄ alkoxy, CO₂R, OH, C₁-C₆ alkoxycarbonyl, CF₃, —(C₁-C₄ alkyl)-NR₁₅C(O)NR₁₆R₁₇, —(C₁-C₄ alkyl)-NR₁₅C(O)R₁₈; wherein
   R₁₅ is H or C₁-C₆ alkyl;
   R₁₆ and R₁₇ are independently H or C₁-C₆ alkyl; or
   R₁₆, R₁₇, and the nitrogen to which they are attached form a morpholinyl ring; and
   R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl.

### EMBODIMENT 24

Compounds according to embodiment 23, wherein
R₅ is of the formula: 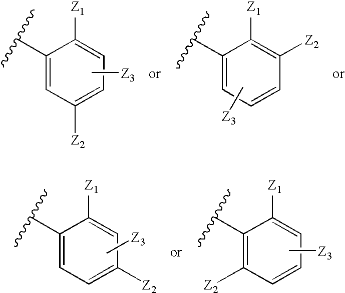
Z₁ is H, halogen, C₁-C₄ alkyl, C₁-C₄ haloalkyl, C₁-C₄ hydroxyalkyl, C₁-C₄ dihydroxyalkyl, or C₁-C₄ alkoxy; and
Z₂ is C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-₄ alkoxy, CO₂R, OH, C₁-C₆ alkoxycarbonyl, or C₁-C₄ haloalkyl;
Z₃ is H, C₁-C₄ alkyl, —C(O)NR₆R₇, —(C₁-C₄ alkyl)-C(O)NR₆R₇, —NR₆R₇NR₆R₇(C₁-C₆ alkyl), C₁-C₆ hydroxyalkyl, C₁-C₆ dihydroxyalkyl, halogen, C₁-C₄ alkoxy, CO₂R, OH, C₁-C₆ alkoxycarbonyl, or C₁-C₄ haloalkyl;
wherein
R₆ and R₇ at each occurrence are independently H, OH, C₁-C₆ alkyl, amino C₁-C₄ alkyl, NH(C₁-C₆ alkyl)alkyl, N(C₁-C₆ alkyl)(C₁-C₆ alkyl) C₁-C₆ alkyl, C₁-C₆ hydroxyalkyl, C₁-C₆ dihydroxyalkyl, C₁-C₆ alkoxy C₁-C₆ alkyl, —SO₂(C₁-C₆ alkyl), —SO₂NH₂, —SO₂NH(C₁-C₆ alkyl), —SO₂N(C₁-C₆ alkyl)(C₁-C₆ alkyl), or C₁-C₆ alkanoyl, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃;
provided that at least one of Z₁, Z₂, and Z₃ is not hydrogen.

### EMBODIMENT 25

Compounds according to embodiment 24, wherein
R₅ is of the formula: 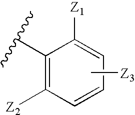
   wherein
Z₁ is H, halogen, C₁-C₄ alkyl, C₁-C₄ haloalkyl, C₁-C₄ hydroxyalkyl, C₁-C₄ dihydroxyalkyl, or C₁-C₄ alkoxy; and
Z₂ is C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, OH, C₁-C₆ alkoxycarbonyl, or C₁-C₄ haloalkyl;
Z₃ is H, C₁-C₄ alkyl, —C(O)NR₆R₇, —(C₁-C₄ alkyl)-C(O)NR₆R₇, —NR₆R7, NR₆R₇(C₁-C₆ alkyl), C₁-C₆ hydroxyalkyl, C₁-C₆ dihydroxyalkyl, halogen, C₁-C₄ alkoxy, CO₂R, OH, C₁-C₆ alkoxycarbonyl, or C₁-C₄ haloalkyl, wherein
   R₆ and R₇ at each occurrence are independently H, OH, C₁-C₆ alkyl, amino C₁-C₄ alkyl, NH(C₁-C₆ alkyl)alkyl, N(C₁-C₆ alkyl)(C₁-C₆ alkyl) C₁-C₆ alkyl, C₁-C₆ hydroxyalkyl, C₁-C₆ dihydroxyalkyl, C₁-C₆ alkoxy C₁-C₆ alkyl, —SO₂(C₁-C₆ alkyl), —SO₂NH₂, —SO₂NH(C₁-C₆ alkyl), —SO₂N(C₁-C₆ alkyl)(C₁-C₆ alkyl), or C₁-C₆ alkanoyl, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃;
provided that at least one of Z₁, Z₂, and Z₃ is not hydrogen.

### EMBODIMENT 26

Compounds according to embodiment 24, wherein
R₅ is of the formula: 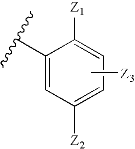
   wherein
Z₁ is H, halogen, C₁-C₄ alkyl, C₁-C₄ haloalkyl, C₁-C₄ hydroxyalkyl, C₁-C₄ dihydroxyalkyl, or C₁-C₄ alkoxy; and
Z₂ is C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, OH, C₁-C₆ alkoxycarbonyl, or C₁-C₄ haloalkyl;
Z₃ is H, C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, OH, C₁-C₆ alkoxycarbonyl, or C₁-C₄ haloalkyl, wherein
   R₆ and R₇ at each occurrence are independently H, OH, C₁-C₆ alkyl, amino C₁-C₄ alkyl, NH(C₁-C₆ alkyl)alkyl, N(C₁-C₆ alkyl)(C₁-C₆ alkyl) C₁-C₆ alkyl, C₁-C₆ hydroxyalkyl, C₁-C₆ dihydroxyalkyl, C₁-C₆ alkoxy C₁-C₆ alkyl, —SO₂(C₁-C₆ alkyl), —SO₂NH₂, —SO₂NH(C₁-C₆ alkyl), —SO₂N(C₁-C₆ alkyl)(C₁-C₆ alkyl), or C₁-C₆ alkanoyl, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃;
provided that at least one of Z₁, Z₂, and Z₃ is not hydrogen.

### EMBODIMENT 27

Compounds according to embodiment 23, wherein
R₅ is either 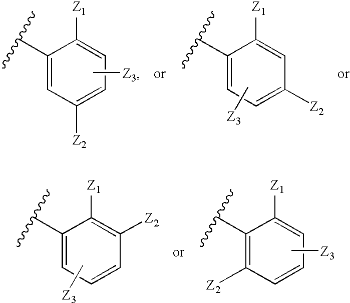
   wherein
Z₁ is H, halogen, C₁-C₄ alkyl, C₁-C₄ haloalkyl, C₁-C₄ hydroxyalkyl, C₁-C₄ dihydroxyalkyl, or C₁-C₄ alkoxy; and
Z₂ is C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, C₁-C₆ alkoxycarbonyl, —(C₁-C₄ alkyl)-NR₁₅C(O)NR₁₆R₁₇, or —(C₁-C₄ alkyl)-NR₁₅C(O)R₁₈;
Z₃ is H, C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, C₁-C₆ alkoxycarbonyl, —(C₁-C₄ alkyl)-NR₁5C(O)NR₁₆R₁₇, or —(C₁-C₄ alkyl)-NR₁₅C(O)R₁₈;
   R₆, R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen;
   R₁₅ is H or C₁-C₆ alkyl;
   R₁₆ and R₁₇ are independently H or C₁-C₆ alkyl; or
   R₁₆, R₁₇, and the nitrogen to which they are attached form a morpholinyl ring;
   R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₃-C₆ alkyl; amino C₁-C₆ alkyl, mono or dialkylamino C₁-C₆ alkyl; provided that at least one of Z₁, Z₂, and Z₃ is not hydrogen.

### EMBODIMENT 28

Compounds according to embodiment 27, wherein
R₅ is of the formula: 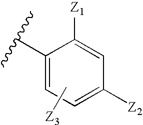
Z₁ is H, halogen, C₁-C₄ alkyl, C₁-C₄ haloalkyl, C₁-C₄ hydroxyalkyl, C₁-C₄ dihydroxyalkyl, or C₁-C₄ alkoxy; and
Z₂ is C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, C₁-C₆ alkoxycarbonyl, —(C₁-C₄ alkyl)-NR₁₅C(O)NR₁₆R₁₇, or —(C₁-C₄ alkyl)-NR₁₅C(O)R₁₈;
Z₃ is H, C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, C₁-C₆ alkoxycarbonyl, —(C₁-C₄ alkyl)-NR₁₅C(O)NR₁₆R₁₇, or —(C₁-C₄ alkyl)-NR₁₅C(O)R₁₈;
   R₆, R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen;
   R₁₅ is H or C₁-C₆ alkyl;
   R₁₆ and R₁₇ are independently H or C₁-C₆ alkyl; or
   R₁₆, R₁₇, and the nitrogen to which they are attached form a morpholinyl ring;
   R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl;
   provided that at least one of Z₁, Z₂, and Z₃ is not hydrogen.

### EMBODIMENT 29. Compounds according to embodiment 27, wherein

R₅ is of the formula: 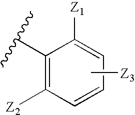
   wherein
Z₁ is H, halogen, C₁-C₄ alkyl C₁-₄ haloalkyl, C₁-₄ hydroxyalkyl, C₁-₄ dihydroxyalkyl, or C₁-₄ alkoxy; and
Z₂ is C₁-₄ alkyl, —C(O)NR₆R₇, —(C₁-₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-₄ alkoxy, CO₂R, C₁-C₆ alkoxycarbonyl, —(C₁-C₄ alkyl)-NR₁₅C(O)NR₁₆R₁₇, or —(C₁-₄ alkyl)-NR₁₅C(O)R₁₈;
Z₃ is H, C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, C₁-C₆ alkoxycarbonyl, —(C₁-C₄ alkyl)-NR₁₅C(O)NR₁₆R₁₇, or —(C₁-₄ alkyl)-NR₁₅C(O)R₁₈;
   R₆, R₇, and the nitrogen to which they are attached form a piperidinyl, pyrrolidinyl, piperazinyl, or a morpholinyl ring, each of which is optionally substituted with 1 or 2 groups that are independently alkyl, hydroxy, hydroxy C₁-₄ alkyl, C₁-₄ dihydroxyalkyl, or halogen;
   R₁₅ is H or C₁-C₆ alkyl;
   R₁₆ and R₁₇ are independently H or C₁-C₆ alkyl; or
   R₁₆, R₁₇, and the nitrogen to which they are attached form a morpholinyl ring;
   R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl;
provided that at least one of Z₁, Z₂, and Z₃ is not hydrogen.

### EMBODIMENT 30

A compound of the formula 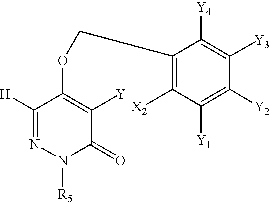
or pharmaceutically acceptable salts thereof, wherein 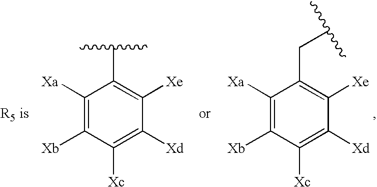
wherein
X₂, Xₐ, X_{b}, X_{c}, X_{d}, and Xₑ are independently selected from —C(O)NR₆R₇, —NR₆R₇, hydroxy(C₁-C₄)alkyl, C₁-C₄ dihydroxyalkyl, H, OH, halogen, haloalkyl, alkyl, haloalkoxy, heteroaryl, heterocycloalkyl, C₃-C₇ cycloalkyl, R₆R₇N—(C₁-C₆ alkyl)-, —CO₂—(C₁-C₆)alkyl, —N(R)C(O)NR₆R₇, —N(R)C(O)-(C₁-C₆)alkoxy, CO₂R—(C₁-C₆ alkyl)-, or —SO₂NR₆R₇; wherein the heteroaryl and heterocycloalkyl groups are optionally substituted with —NR₆R₇, —C(O)NR₆R₇, R₆R₇N—(C₁-C₆ alkyl)-, C₁-C₆ alkyl, C₁-C₆ alkoxy, or halogen; or
R₅ is heteroaryl or heteroarylalkyl, wherein the heteroaryl and heteroaryl groups are optionally substituted with 1,2, 3, or 4 groups that are independently —C(O)NR₆R₇, —NR₆R₇, hydroxy(C₁-C₄)alkyl, C₁-C₄ dihydroxyalkyl, H, OH, halogen, haloalkyl, alkyl, haloalkoxy, R₆R₇N—(C₁-C₆ alkyl)-, —CO₂—(C₁-C₆)alkyl, —N(R)C(O)NR₆R₇, or —N(R)C(O)—(C₁-C₆)alkoxy; wherein
   R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₄ dihydroxyalkyl, C₁-C₆ thiohydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, SH, NH₂, NH(alkyl), N(alkyl)(alkyl), —O—C₁-C₄ alkanoyl, C₁-C₄ alkyl, CF₃, or OCF₃; or
   R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen;
   R at each occurrence is independently H or C₁-C₆ alkyl; and
Y, Y₁, Y₂, Y₃, and Y₄ are independently selected from H, halogen, alkyl, carboxaldehyde, hydroxyalkyl, dihydroxyalkyl, alkenyl, alkynyl, CN, alkanoyl, alkoxy, alkoxyalkyl, haloalkyl, and carboxyl.

### EMBODIMENT 31

Compounds according to embodiment 30, wherein
R₅ is

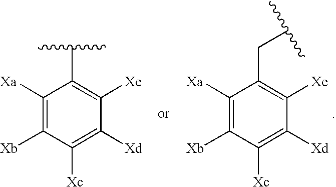

### EMBODIMENT 32

Compounds according to embodiment 31, wherein
Y₂, Y₄, and Y are independently halogen; and
Y₁ and Y₃ are both hydrogen.

### EMBODIMENT 33

Compounds according to embodiment 32, wherein
R₅ is 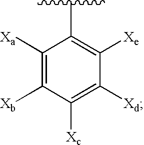
X₂ is H, methyl, NR₆R₇, R₆R₇N—(C₁-C₆ alkyl)-, —C(O)NR₆R₇, C₁-C₆ hydroxyalkyl, C₁-C₆ dihydroxyalkyl, or —(C₁-C₄ alkyl)-morpholinyl; and
Xₐ and Xₑ are independently halogen, NH₂, NH(C₁-C₆ alkyl), N(C₁-C₆ alkyl)(C₁-C₆ alkyl), methyl, or hydrogen; provided that one of Xₐ and Xₑ is not hydrogen.

### EMBODIMENT 34

Compounds according to embodiment 33, wherein
one of X_{b} and X_{c} is hydrogen and the other is —NR₆R₇, R₆R₇N—(C₁-C₆ alkyl)-, —C(O)NR₆R₇, —SO₂NR₆R₇, or halogen; where
   R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, SH, NH₂, NH(alkyl), N(alkyl)(alkyl), —O—C₁-C₄ alkanoyl, C₁-C₄ alkyl, CF₃, or OCF₃; or
   R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen.

### EMBODIMENT 35

Compounds according to embodiment 34, wherein
R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, NH₂, NH(alkyl), N(alkyl)(alkyl), —O—C₁-C₄ alkanoyl, C₁-C₄ alkyl, CF₃, or OCF₃.

### EMBODIMENT 36

Compounds according to embodiment 35, wherein
Xₐ is hydrogen, methyl, fluorine, or chlorine;
X_{c} and X_{d} are both hydrogen;
X_{b} is —NR₆R₇, R₆R₇N—(C₁-C₆ alkyl)-, —C(O)NR₆R₇; wherein
R₆ and R₇ are independently at each occurrence H, C₁-C₆ alkyl, C₁-C₆ hydroxyalkyl, C₁-C₄ dihydroxyalkyl, C₁-C₆ alkoxy, C₁-C₆ alkoxy C₁-C₆ alkyl, or C₁-C₆ alkanoyl, wherein each of the above is optionally substituted with 1, 2, or 3 groups that are independently NH₂, NH(C₁-C₆ alkyl), N(C₁-C₆ alkyl)(C₁-C₆ alkyl), OH, SH, halogen, or C₃-C₆ cycloalkyl.

### EMBODIMENT 36a

Compounds according to embodiment 36, wherein
Xₐ is hydrogen;
X_{b} is —NR₆R₇, or —C(O)NR₆R₇;
X_{c} and X_{d} are both hydrogen.

### EMBODIMENT 36b

A compound according to embodiment 36a, wherein
Xₑ is halogen, NH₂, NH(C₁-C₆ alkyl), N(C₁-C₆ alkyl)(C₁-C₆ alkyl), or methyl.

### EMBODIMENT 36c

A compound according to embodiment 36b, wherein Xₑ is halogen or methyl. More preferably, Xₑ is methyl.

### EMBODIMENT 36d

A compound according to embodiment 36c, wherein
R₆ and R₇ are independently at each occurrence H, C₁-C₆ alkyl, C₁-C₆ hydroxyalkyl, C₁-C₆ alkoxy, or C₁-C₆ alkanoyl, wherein each of the above is optionally substituted with 1 or 2 groups that are independently NH₂, NH(C₁-C₆ alkyl), N(C₁-C₆ alkyl)(C₁-C₆ alkyl), OH, SH, or halogen.

### EMBODIMENT 36e

A compound according to embodiment 36d, wherein
R₆ is H.

### EMBODIMENT 37

Compounds according to embodiment 32, wherein
R₅ is 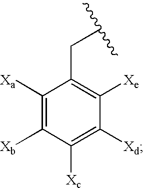
Xₐ is H, fluoro, chloro, or methyl;
Xₑ is hydrogen, halogen, or methyl; and
X_{b} is H;
X_{d} is H or halogen;

### EMBODIMENT 38

Compounds according to embodiment 37, wherein
X_{c} is —SO₂NR₆R₇, or halogen; wherein
   R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, SH, NH₂, NH(alkyl), N(alkyl)(alkyl), —O—C₁-C₄ alkanoyl, C₁-C₄ alkyl, CF₃, or OCF₃; or
   R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen; or
X_{c} is fluoro, chloro, —NH₂, —NH(C₁-C₆ alkyl), —N(C₁-C₆ alkyl)(C₁-C₆ alkyl), —SO₂NH₂, —SO₂NH(C₁-C₆ alkyl), —SO₂N(C₁-C₆ alkyl)(C₁-C₆ alkyl), or piperazinyl, wherein the piperazinyl group is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen.

### EMBODIMENT 39

Compounds according to embodiment 37, wherein
X_{c} is —C(O)NR₆R₇, —(C₁-C₆ alkyl)-C(O)NR₆R₇, —NR₆R₇, or R₆R₇N—(C₁-C₆ alkyl)-; wherein
   R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, —NH₂, —NH(alkyl), —N(alkyl)(alkyl), —O—C₁-C₄ alkanoyl, C₁-C₄ alkyl, CF₃, or OCF₃; or
   R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen.

### EMBODIMENT 40

Compounds according to embodiment 39, wherein
R₆ is hydrogen; and
R₇ is C₁-C₆ alkyl or C₁-C₆ alkanoyl, each of which is optionally substituted with 1, 2, or 3 groups that are independently NH₂, NH(C₁-C₆ alkyl), N(C₁-C₆ alkyl)(C₁-C₆ alkyl), OH, SH, cyclopropyl, or C₁-C₄ alkoxy;

### EMBODIMENT 41

Compounds according to embodiment 40, wherein
X_{c} is —C(O)NR₆R₇.

### EMBODIMENT 42

Compounds according to embodiment 40, wherein
X_{c} is NR₆R₇, or R₆R₇N—(C₁-C₆ alkyl)-.

### EMBODIMENT 43

Compounds according to embodiment 31, wherein
Xₐ is hydrogen;
two of X_{b}, X_{c}, and X_{d} are hydrogen and the other is —C(O)NR₆R₇, —(C₁-C₆ alkyl)-C(O)NR₆R₇, —NR₆R₇, R₆R₇N—(C₁-C₆ alkyl)- or —CO₂-(C₁-C₆)alkyl; wherein
   R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, NH₂, NH(alkyl), N(alkyl)(alkyl), —O—C₁-C₄ alkanoyl, C₁-C₄ alkyl, CF₃, or OCF₃; or
   R₆, R₇, and the nitrogen to which they are attached form a morpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen; and
Xₑ is hydrogen, methyl, C₁-C₂ alkoxy, or halogen.

### EMBODIMENT 44

Compounds according to embodiment 43, wherein
X_{b} is —C(O)NR₆R₇, —(C₁-C₆ alkyl)-C(O)NR₆R₇, —NR₆R₇, or R₆R₇N—(C₁-C₆ alkyl)-wherein
R₆ is hydrogen or C₁-₄ alkyl;
R₇ is H, OH, C₁-C₆ alkyl or C₁-C₆ alkanoyl, wherein the alkyl and alkanoyl groups substituted with 1, 2, or 3 groups that are independently NH₂, NH(C₁-C₆ alkyl), N(C₁-C₆ alkyl)(C₁-C₆ alkyl), C₃-C₆ cycloalkyl, OH, or C₁-C₄ alkoxy.

### EMBODIMENT 44a

Compounds according to embodiment 44, wherein
R₅ is of the formula:

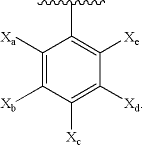

### EMBODIMENT 44b

A compound according to embodiment 44a, wherein
Xₐ is hydrogen;
X_{b} is —NR₆R₇, or —C(O)NR₆R₇; and
X_{c} and X_{d} are both hydrogen.

### EMBODIMENT 44c

A compound according to embodiment 44b, wherein
Xₑ is methyl, C₁-C₂ alkoxy, or halogen.

### EMBODIMENT 44d

A compound according to embodiment 44c, wherein Xₑ is halogen or methyl. More preferably, Xₑ is methyl.

### EMBODIMENT 44e

A compound according to embodiment 44d, wherein
R₆ is hydrogen or C₁-C₄ alkyl; and
R₇ is OH, C₁-C₆ alkyl or C₁-C₆ alkanoyl, wherein the alkyl and alkanoyl groups substituted with 1, 2, or 3 groups that are independently NH₂, NH(C₁-C₆ alkyl), N(C₁-C₆ alkyl)(C₁-C₆ alkyl), C₃-C₆ cycloalkyl, OH, or C₁-C₄ alkoxy.

### EMBODIMENT 44f

A compound according to embodiment 43, wherein
R₇ is H, C₁-C₆ alkyl, or C₁-C₆ alkoxy.

### EMBODIMENT 44g

A compound according to embodiment 44f, wherein
R₆ is hydrogen or C₁-C₄ alkyl; and
R₇ is C₁-C₄ alkyl, or C₁-C₄ alkoxy.

### EMBODIMENT 45

Compounds according to embodiment 31, wherein
X₁ is halogen or methyl;
X_{b} is H, —NR₆R₇, R₆R₇N—(C₁-C₆ alkyl)-, —C(O)NR₆R₇, or —CO₂—(C₁-C₆)alkyl;
X_{c} is —NR₆R₇, R₆R₇N—(C₁-C₆ alkyl)-, —C(O)NR₆R₇, halogen, —CO₂—(C₁-C₆)alkyl, NH₂, NH(C₁-C₆ alkyl), N(C₁-C₆ alkyl)(C₁-C₆ alkyl), —SO₂NH₂, —SO₂NH(C₁-C₆ alkyl), —SO₂N(C₁-C₆ alkyl)(C₁-C₆ alkyl), or piperazinyl, wherein the piperazinyl group is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen;
X_{d} is hydrogen;
Xₑ is H, methyl, NH₂, NH(C₁-C₆ alkyl) or N(C₁-C₆ alkyl)(C₁-C₆ alkyl).

### EMBODIMENT 46

Compounds according to embodiment 31, wherein
X₂, Xₐ, X_{b}, X_{c}, X_{d}, and Xₑ are independently selected from H, OH, halogen, CF₃, alkyl, OCF₃, pyridyl, pyridazinyl, pyrimidyl, pyrazinyl, thienyl, furyl, pyrrolyl, piperidinyl, piperazinyl, or C₃-C₇ cycloalkyl, wherein each of the above is optionally substituted with —NR₆R₇, —C(O)NR₆R₇, R₆R₇N—(C₁-C₆ alkyl)-, C₁-C₆ alkyl, C₁-C₆ alkoxy, or halogen.

### EMBODIMENT 47

Compounds according to embodiment 30, wherein
R₅ is a heteroaryl or heteroarylalkyl group, where each heteroaryl is pyridyl, pyridazinyl, pyrimidinyl, pyrazinyl, pyrazolyl, imidazolyl, dihydroindolyl, dihydroisoindolyl, indolon-2-yl, quinolinyl, isoquinolinyl, tetrahydroisoquinolinyl, dihydroisoquinolinyl, or indolyl, each of which is optionally substituted with 1, 2, 3, or 4 groups that are independently —C(O)NR₆R₇, —NR₆R₇, hydroxy(C₁-C₄)alkyl, C₁-C₄ dihydroxyalkyl, hydrogen, hydroxy, halogen, haloalkyl, alkyl, haloalkoxy, R₆R₇N—(C₁-C₆ alkyl)-, —CO₂—(C₁-C₆)alkyl, —N(R)C(O)NR₆R₇, or —N(R)C(O)—(C₁-C₆)alkoxy; wherein
   R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, C₁-C₆ thiohydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, SH, NH₂, NH(alkyl), N(alkyl)(alkyl), —O—C₁-C₄ alkanoyl, C₁-C₄ alkyl, CF₃, or OCF

### EMBODIMENT 48

Compounds according to embodiment 47, wherein
Y₂, Y₄, and Y are independently halogen; and
Y₁ and Y₃ are both hydrogen.

### EMBODIMENT 49

Compounds according to embodiment 48, wherein
X₂ is H, methyl, —NR₆R₇, R₆R₇N—(C₁-C₆ alkyl)-, —C(O)NR₆R₇, C₁-C₆ hydroxyalkyl, C₁-C₆ dihydroxyalkyl, or —(C₁-C₄ alkyl)-morpholinyl.

### EMBODIMENT 50

Compounds according to embodiment 49, wherein
R₅ is pyridyl C₁-C₆ alkyl, pyrimidinyl C₁-C₆ alkyl, or pyrazinyl C₁-C₆ alkyl, each of which is optionally substituted with 1, 2, or 3 groups that are independently hydroxy(C₁-C₄)alkyl, C₁-C₄ dihydroxyalkyl, OH, halogen, CF₃, (C₁-C₄)alkyl, OCF₃, —NR₆R₇, R₆R₇N—(C₁-C₆ alkyl)-, or —C(O)NR₆R₇.

### EMBODIMENT 51

Compounds according to embodiment 50, wherein
R₅ is of the formula: 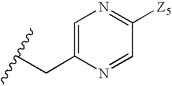
   wherein
Z₅ is hydroxy(C₁-C₄)alkyl, C₁-C₄ dihydroxyalkyl, OH, halogen, CF₃, (C₁-C₄)alkyl, OCF₃, —NR₆R₇, R₆R₇N—(C₁-C₆ alkyl)-, or —C(O)NR₆R₇, wherein
   R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy.

### EMBODIMENT 52

Compounds according to embodiment 50, wherein
R₅ is of the formula: 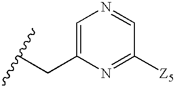
   wherein
Z₅ is hydroxy(C₁-C₄)alkyl, C₁-C₄ dihydroxyalkyl, OH, halogen, CF₃, (C₁-C₄)alkyl, OCF₃, —NR₆R₇, R₆R₇N—(C₁-C₆ alkyl)-, or —C(O)NR₆R₇, wherein
   R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy.

### EMBODIMENT 53

Compounds according to embodiment 50, wherein
R₅ is of the formula: 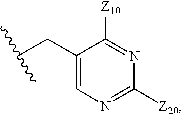
   wherein
Z₁₀ is H or methyl; and
Z₂₀ is hydroxy(C₁-C₄)alkyl, C₁-C₄ dihydroxyalkyl, OH, halogen, CF₃, (C₁-C₄)alkyl, OCF₃, —NR₆R₇, R₆R₇N—(C₁-C₆ alkyl)-, or —C(O)NR₆R₇, wherein
   R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy.

The invention also provides methods of treating a TNF mediated disorder, a p38 kinase-alpha mediated disorder, inflammation and/or arthritis in a subject, the method comprising treating a subject having or susceptible to such disorder or condition with a compound of the formula I, or a pharmaceutically acceptable salt thereof.

The methods of the invention are useful for treating or preventing inflammation; arthritis, rheumatoid arthritis, spondylarthropathies, gouty arthritis, osteoarthritis, systemic lupus erthematosus, juvenile arthritis; neuroinflammation; pain, neuropathic pain; fever; pulmonary disorders, lung inflammation, adult respiratory distress syndrome, pulmonary sarcoisosis, asthma, silicosis, chronic pulmonary inflammatory disease; cardiovascular disease, arteriosclerosis, myocardial infarction, thrombosis, congestive heart failure, cardiac reperfusion injury; cardiomyopathy; reperfusion injury; renal reperfusion injury; ischemia including stroke and brain ischemia; brain trauma; brain edema; liver disease and nephritis; gastrointestinal conditions, inflammatory bowel disease, Crohn's disease, gastritis, irritable bowel syndrome, ulcerative colitis; ulceratiuve diseases, gastric ulcers; ophthalmic diseases, retinitis, retinopathies, uveitis, ocular photophobia, acute injury to the eye tissue; ophthalmological conditions, corneal graft rejection, ocular neovascularization, retinal neovascularization, neovascularization following injury or infection, diabetic retinopathy, retrolental fibroplasias, neovascular glaucoma; diabetes; diabetic nephropathy; skin-related conditions, psoriasis, eczema, burns, dermatitis, keloid formation, scar tissue formation, angiogenic disorders; viral and bacterial infections, sepsis, septic shock, gram negative sepsis, malaria, meningitis, opportunistic infections, cachexia secondary to infection or malignancy, cachexia secondary to acquired immune deficiency syndrome (AIDS), AIDS, ARC (AIDS related complex), pneumonia, herpes virus; myalgias due to infection; influenza; endotoxic shock; toxic shock syndrome; autoimmune disease, graft vs. host reaction and allograft rejections; treatment of bone resorption diseases, osteoporosis; multiple sclerosis; disorders of the female reproductive system, endometriosis; hemaginomas, infantile hemagionmas, angiofibroma of the nasopharynx, avascular necrosis of bone; benign and malignant tumors/neoplasia, cancer, colorectal cancer, brain cancer, bone cancer, epithelial call-derived neoplasia (epithelial carcinoma), basal cell carcinoma, adenocarcinoma, gastrointestinal cancer, lip cancer, mouth cancer, esophageal cancer, small bowel cancer, stomach cancer, colon cancer, liver cancer, bladder cancer, pancreas cancer, ovarian cancer, cervical cancer, lung cancer, breast cancer, skin cancer, squamus cell and/or basal cell cancers, prostate cancer, renal cell carcinoma, and other known cancers that affect epithelial cells throughout the body; leukemia; lymphoma; systemic lupus erthrematosis (SLE); angiogenesis including neoplasia; metastasis; central nervous system disorders, central nervous system disorders having an inflammatory or apoptotic component, Alzheimer's disease, Parkinson's disease, Huntington's disease, amyotrophic lateral sclerosis, spinal cord injury, and peripheral neuropathy.

Representative compounds of formula I include:
2-benzyl-5-(benzyloxy)-4-bromopyridazin-3(2H)-one;
2-benzyl-4-bromo-5-[(4-fluorobenzyl)oxy]pyridazin-3(2H)-one;
2-benzyl-4-chloro-5-methoxypyridazin-3(2H)-one;
1-benzyl-6-oxo-1,6-dihydropyridazine-3-carboxylic acid;
4,5-dibromo-2-(2,6-dichlorophenyl)pyridazin-3(2H)-one;
2-benzyl-4,5-dibromopyridazin-3(2H)-one;
4,5-dibromo-2-phenylpyridazin-3(2H)-one;
2-benzyl-4-bromo-5-[(4-fluorobenzyl)amino]pyridazin-3(2H)-one;
2-benzyl-4-bromo-5-[(4-fluorophenyl)thio]pyridazin-3(2H)-one;
4-bromo-2-(2,6-dichlorophenyl)-5-[(4-fluorobenzyl)oxy]pyridazin-3(2H)-one;
4-bromo-2-(2,6-dichlorophenyl)-5-[(4-fluorobenzyl)amino]pyridazin-3(2H)-one;
4-bromo-2-(2,6-dichlorophenyl)-5-[(4-fluorophenyl)thio]pyridazin-3(2H)-one;
2-benzyl-4-bromo-5-[(2-isopropylphenyl)thio]pyridazin-3(2H)-one;
2-benzyl-4-bromo-5-[(tetrahydrofuran-2-ylmethyl)amino]pyridazin-3(2H)-one;
[(1-benzyl-5-bromo-6-oxo-1,6-dihydropyridazin-4-yl)amino](phenyl)acetic acid;
2-benzyl-4-bromo-5-(phenethyloxy)pyridazin-3(2H)-one;
2-benzyl-5-(benzyloxy)pyridazin-3(2H)-one;
5-anilino-4-bromo-2-(2,6-dichlorophenyl)pyridazin-3(2H)-one;
4-bromo-2-(2,6-dichlorophenyl)-5-{[(1s)-1-phenylethyl]amino}pyridazin-3(2H)-one;
4-bromo-2-(2,6-dichlorophenyl)-5-{[(1R)-1-phenylethyl]amino}pyridazin-3(2H)-one;
4-bromo-2-(2,6-dichlorophenyl)-5-[(1-methyl-1-phenylethyl)amino]pyridazin-3(2H)-one;
{[5-bromo-1-(2,6-dichlorophenyl)-6-oxo-1,6-dihydropyridazin-4-yl]amino}(phenyl)acetic acid;
ethyl {[5-bromo-1-(2,6-dichlorophenyl)-6-oxo-1,6-dihydropyridazin-4-yl]amino}(phenyl)acetate;
4-bromo-5-[(2-chlorobenzyl)amino]-2-(2,6-dichlorophenyl)pyridazin-3(2H)-one;
4-bromo-5-[(3-chlorobenzyl)amino]-2-(2,6-dichlorophenyl)pyridazin-3(2H)-one;
4-bromo-2-(2,6-dichlorophenyl)-5-[(tetrahydrofuran-2-ylmethyl)amino]pyridazin-3(2H)-one;
4-bromo-2-(2,6-dichlorophenyl)-5-[(2-phenylethyl)amino]pyridazin-3(2H)-one;
4-bromo-2-(2,6-dichlorophenyl)-5-{[(1R,2S)-2-phenylcyclopropyl]amino}pyridazin-3(2H)-one;
2-benzyl-4-bromo-5-phenoxypyridazin-3(2H)-one;
5-anilino-2-benzyl-4-bromopyridazin-3(2H)-one;
5-[benzyl(methyl)amino]-4-bromo-2-(2,6-dichlorophenyl)pyridazin-3(2H)-one;
N-benzyl-N-[5-bromo-1-(2,6-dichlorophenyl)-6-oxo-1,6-dihydropyridazin-4-yl]acetamide;
N,1-dibenzyl-6-oxo-1,6-dihydropyridazine-3-carboxamide;
2-benzyl-6-(hydroxymethyl)pyridazin-3(2H)-one;
1-benzyl-6-oxo-N-(2-phenylethyl)-1,6-dihydropyridazine-3-carboxamide;
1-benzyl-N-(4-fluorobenzyl)-6-oxo-1,6-dihydropyridazine-3-carboxamide;
benzyl 1-benzyl-6-oxo-1,6-dihydropyridazine-3-carboxylate;
2-benzyl-6-(3-phenylpropanoyl)pyridazin-3(2H)-one;
2-benzyl-6-{[(2-phenylethyl)amino]methyl}pyridazin-3(2H)-one;
2-benzyl-6-[(2-phenylethoxy)methyl]pyridazin-3(2H)-one;
2-benzyl-6-(4-phenylbutyl)pyridazin-3(2H)-one;
2-benzyl-6-[3-(4-fluorophenyl)propyl]pyridazin-3(2H)-one;
2-benzyl-6-{[(4-fluorobenzyl)oxy]methyl}pyridazin-3(2H)-one;
2-benzyl-6-{[(4-fluorobenzyl)amino]methyl}pyridazin-3(2H)-one;
1-benzyl-5-methyl-6-oxo-1,6-dihydropyridazine-3-carboxamide;
1-benzyl-5-ethyl-6-oxo-1,6-dihydropyridazine-3-carboxamide;
1-benzyl-5-isopropyl-6-oxo-1,6-dihydropyridazine-3-carboxamide;
4-bromo-2-(3,5-dichloropyridin-4-yl)-5-[(2,4-difluorobenzyl)oxy]pyridazin-3(2H)-one;
4-bromo-2-(2,6-dichlorophenyl)-5-[(2,3,4-trifluorobenzyl)oxy]pyridazin-3(2H)-one;
4-bromo-2-(2,6-dichlorophenyl)-5-[(2,4,6-trifluorobenzyl)oxy]pyridazin-3(2H)-one;
4-bromo-2-(2,6-dichlorophenyl)-5-{[2-(hydroxymethyl)benzyl]oxy}pyridazin-3(2H)-one;
4-bromo-2-(3,5-dichloropyridin-4-yl N-oxide)-5-[(2,4-difluorobenzyl)oxy]pyridazin-3(2H)-one;
2-({[5-bromo-1-(2,6-dichlorophenyl)-6-oxo-1,6-dihydropyridazin-4-yl]oxy}methyl)benzyl methanesulfonate;
4-bromo-2-(2,6-dichlorophenyl)-5-{[2-(2-fluorophenyl)ethyl]amino}pyridazin-3(2H)-one;
2-benzyl-4-bromo-5-(2-phenylethoxy)pyridazin-3(2H)-one;
5-(benzyloxy)-4-bromo-2-phenylpyridazin-3(2H)-one;
5-(benzylamino)-4-bromo-2-(2,6-dichlorophenyl)pyridazin-3(2H)-one;
4-bromo-2-(2,6-dichlorophenyl)-5-(2-phenylethoxy)pyridazin-3(2H)-one;
5-(benzyloxy)-4-bromo-2-(2,6-dichlorophenyl)pyridazin-3(2H)-one;
4-bromo-2-(2,6-dichlorophenyl)-5-[(2-hydroxy-2-phenylethyl)amino]pyridazin-3(2H)-one;
4-bromo-2-(2,6-dichlorophenyl)-5-{[(1R,2S)-2-hydroxy-1-methyl-2-phenylethyl]amino}pyridazin-3(2H)-one;
4-bromo-2-(2,6-dichlorophenyl)-5-{[(1S,2R)-2-hydroxy-1-methyl-2-phenylethyl]amino}pyridazin-3(2H)-one;
5-[(l-benzyl-2-hydroxyethyl)amino]-4-bromo-2-(2,6-dichlorophenyl)pyridazin-3(2H)-one;
4-bromo-2-(2,6-dichlorophenyl)-5-{[(1S)-2-hydroxy-1-phenylethyl]amino}pyridazin-3(2H)-one;
4-bromo-2-(2,6-dichlorophenyl)-5-{[(1R)-2-hydroxy-1-phenylethyl]amino}pyridazin-3(2H)-one;
4-bromo-2-(2,6-dichlorophenyl)-5-[methyl(2-phenylethyl)amino]pyridazin-3(2H)-one;
4-bromo-2-(2,6-dichlorophenyl)-5-[(2-hydroxyethyl)(2-phenylethyl)amino]pyridazin-3(2H)-one;
5-[(2-aminobenzyl)amino]-4-bromo-2-(2,6-dichlorophenyl)pyridazin-3(2H)-one;
4-bromo-2-(2,6-dichlorophenyl)-5-[4-(4-fluorophenyl)piperazin-1-yl]pyridazin-3(2H)-one;
4-bromo-2-(2,6-dichlorophenyl)-5-[(2-methoxybenzyl)oxy]pyridazin-3(2H)-one;
4-bromo-2-(2,6-dichlorophenyl)-5-(3-phenylpropoxy)pyridazin-3(2H)-one;
4-bromo-2-(2,6-dichlorophenyl)-5-(2-pyridin-2-ylethoxy)pyridazin-3(2H)-one;
4-bromo-2-(2,6-dichlorophenyl)-5-hydroxypyridazin-3(2H)-one;
4-{[5-bromo-l-(2,6-dichlorophenyl)-6-oxo-1,6-dihydropyridazin-4-yl]amino}-3-(4-chlorophenyl)butanoic acid;
4-bromo-5-{[2-(4-chlorophenyl)-4-hydroxybutyl]amino}-2-(2,6-dichlorophenyl)pyridazin-3(2H)-one;
4-bromo-5-{[2-(chloromethyl)benzyl]oxy}-2-(2,6-dichlorophenyl)pyridazin-3(2H)-one;
5-(1-benzylhydrazino)-4-bromo-2-(2,6-dichlorophenyl)pyridazin-3(2H)-one;
4-bromo-2-(2,6-dichlorophenyl)-5-[(2,4-difluorobenzyl)oxy]pyridazin-3(2H)-one;
4-bromo-2-(2,6-dichlorophenyl)-5-[(3,4-difluorobenzyl)oxy]pyridazin-3(2H)-one;
2-(2,6-dichlorophenyl)-5-(2-phenylethoxy)pyridazin-3(2H)-one;
2-(2,6-dichlorophenyl)-4-methyl-5-(2-phenylethoxy)pyridazin-3(2H)-one;
2-(2,6-dichlorophenyl)-4-phenyl-5-(2-phenylethoxy)pyridazin-3(2H)-one;
2-(2,6-dichlorophenyl)-4-methoxy-5-(2-phenylethoxy)pyridazin-3(2H)-one;
2-(2,6-dichlorophenyl)-4-isobutyl-5-(2-phenylethoxy)pyridazin-3(2H)-one;
2-(2,6-dichlorophenyl)-4-phenoxy-5-(2-phenylethoxy)pyridazin-3(2H)-one;
4-bromo-5-(2-phenylethoxy)pyridazin-3(2H)-one;
4-bromo-5-(2-phenylethoxy)-2-(pyridin-4-ylmethyl)pyridazin-3(2H)-one;
4-bromo-2-[2-(dimethylamino)ethyl]-5-(2-phenylethoxy)pyridazin-3(2H)-one;
4-bromo-2-[3-(dimethylamino)propyl]-5-(2-phenylethoxy)pyridazin-3(2H)-one;
4-bromo-2-(2-hydroxyethyl)-5-(2-phenylethoxy)pyridazin-3(2H)-one;
4-bromo-5-[(2,4-difluorobenzyl)oxy]pyridazin-3(2H)-one;
4-bromo-5-[(2,4-difluorobenzyl)oxy]-2-(pyridin-4-ylmethyl)pyridazin-3(2H)-one;
4-bromo-5-[(2,4-difluorobenzyl)oxy]-2-[2-(dimethylamino)ethyl]pyridazin-3(2H)-one;
4-bromo-5-[(2,4-difluorobenzyl)oxy]-2-[3-(dimethylamino)propyl]pyridazin-3(2H)-one;
4-bromo-5-[(2,4-difluorobenzyl)oxy]-2-(2-hydroxyethyl)pyridazin-3(2H)-one;
4-bromo-5-[(2,4-difluorobenzyl)oxy]-2-[(2-methyl-1,3-thiazol-4-yl)methyl]pyridazin-3(2H)-one;
and the pharmaceutically acceptable salts thereof.

Preferred compounds of formula I include:
2-benzyl-4-bromo-5-[(4-fluorobenzyl)oxy]pyridazin-3(2H)-one;
2-benzyl-4-chloro-5-methoxypyridazin-3(2H)-one;
4,5-dibromo-2-(2,6-dichlorophenyl)pyridazin-3(2H)-one;
2-benzyl-4,5-dibromopyridazin-3(2H)-one;
4,5-dibromo-2-phenylpyridazin-3(2H)-one;
2-benzyl-4-bromo-5-[(4-fluorobenzyl)amino]pyridazin-3(2H)-one;
4-bromo-2-(2,6-dichlorophenyl)-5-[(4-fluorobenzyl)oxy]pyridazin-3(2H)-one;
4-bromo-2-(2,6-dichlorophenyl)-5-[(4-fluorobenzyl)amino]pyridazin-3(2H)-one;
2-benzyl-4-bromo-5-(phenethyloxy)pyridazin-3(2H)-one;
2-benzyl-5-(benzyloxy)pyridazin-3(2H)-one;
4-bromo-2-(2,6-dichlorophenyl)-5-[(1-methyl-1-phenylethyl)amino]pyridazin-3(2H)-one;
ethyl {[5-bromo-1-(2,6-dichlorophenyl)-6-oxo-1,6-dihydropyridazin-4-yl]amino}(phenyl)acetate;
4-bromo-5-[(2-chlorobenzyl)amino]-2-(2,6-dichlorophenyl)pyridazin-3(2H)-one;
4-bromo-5-[(3-chlorobenzyl)amino]-2-(2,6-dichlorophenyl)pyridazin-3(2H)-one;
4-bromo-2-(2,6-dichlorophenyl)-5-[(2-phenylethyl)amino]pyridazin-3(2H)-one;
5-[benzyl(methyl)amino]-4-bromo-2-(2,6-dichlorophenyl)pyridazin-3(2H)-one;
4-bromo-2-(3,5-dichloropyridin-4-yl)-5-[(2,4-difluorobenzyl)oxy]pyridazin-3(2H)-one;
4-bromo-2-(2,6-dichlorophenyl)-5-[(2,3,4-trifluorobenzyl)oxy]pyridazin-3(2H)-one;
4-bromo-2-(2,6-dichlorophenyl)-5-[(2,4,6-trifluorobenzyl)oxy]pyridazin-3(2H)-one;
4-bromo-2-(2,6-dichlorophenyl)-5-{[2-(hydroxymethyl)benzyl]oxy}pyridazin-3(2H)-one;
4-bromo-2-(3,5-dichloropyridin-4-yl N-oxide)-5-[(2,4-difluorobenzyl)oxy]pyridazin-3(2H)-one;
2-({[5-bromo-1-(2,6-dichlorophenyl)-6-oxo-1,6-dihydropyridazin-4-yl]oxy}methyl)benzyl methanesulfonate;
4-bromo-2-(2,6-dichlorophenyl)-5-{[2-(2-fluorophenyl)ethyl]amino}pyridazin-3(2H)-one;
2-benzyl-4-bromo-5-(2-phenylethoxy)pyridazin-3(2H)-one;
5-(benzyloxy)-4-bromo-2-phenylpyridazin-3(2H)-one;
5-(benzylamino)-4-bromo-2-(2,6-dichlorophenyl)pyridazin-3(2H)-one;
4-bromo-2-(2,6-dichlorophenyl)-5-(2-phenylethoxy)pyridazin-3(2H)-one;
5-(benzyloxy)-4-bromo-2-(2,6-dichlorophenyl)pyridazin-3(2H)-one;
4-bromo-2-(2,6-dichlorophenyl)-5-[(2-hydroxy-2-phenylethyl)amino]pyridazin-3(2H)-one;
4-bromo-2-(2,6-dichlorophenyl)-5-{[(1R,2S)-2-hydroxy-1-methyl-2-phenylethyl]amino}pyridazin-3(2H)-one;
4-bromo-2-(2,6-dichlorophenyl)-5-{[(1S,2R)-2-hydroxy-1-methyl-2-phenylethyl]amino}pyridazin-3(2H)-one;
5-[(l-benzyl-2-hydroxyethyl)amino]-4-bromo-2-(2,6-dichlorophenyl)pyridazin-3(2H)-one;
4-bromo-2-(2,6-dichlorophenyl)-5-{[(1S)-2-hydroxy-1-phenylethyl]amino}pyridazin-3(2H)-one;
3-[5-bromo-4-[(2,4-difluorobenzyl)oxy]-6-oxopyridazin-1(6H)-yl]-4-methylbenzamide;
4-bromo-2-(2,6-dichlorophenyl)-5-[methyl(2-phenylethyl)amino]pyridazin-3(2H)-one;
4-bromo-2-(2,6-dichlorophenyl)-5-[(2-hydroxyethyl)(2-phenylethyl)amino]pyridazin-3(2H)-one;
5-[(2-aminobenzyl)amino]-4-bromo-2-(2,6-dichlorophenyl)pyridazin-3(2H)-one;
3-[5-bromo-4-[(2,4-difluorobenzyl)oxy]-6-oxopyridazin-1(6H)-yl]-N,4-dimethylbenzamide;
4-bromo-2-(2,6-dichlorophenyl)-5-[4-(4-fluorophenyl)piperazin-1-yl]pyridazin-3(2H)-one;
4-bromo-2-(2,6-dichlorophenyl)-5-[(2-methoxybenzyl)oxy]pyridazin-3(2H)-one;
4-bromo-2-(2,6-dichlorophenyl)-5-(3-phenylpropoxy)pyridazin-3(2H)-one;
4-bromo-2-(2,6-dichlorophenyl)-5-(2-pyridin-2-ylethoxy)pyridazin-3(2H)-one;
4-bromo-2-(2,6-dichlorophenyl)-5-hydroxypyridazin-3(2H)-one;
4-{[5-bromo-l-(2,6-dichlorophenyl)-6-oxo-1,6-dihydropyridazin-4-yl]amino}-3-(4-chlorophenyl)butanoic acid;
4-bromo-5-{[2-(chloromethyl)benzyl]oxy}-2-(2,6-dichlorophenyl)pyridazin-3(2H)-one;
5-(1-benzylhydrazino)-4-bromo-2-(2,6-dichlorophenyl)pyridazin-3(2H)-one;
4-bromo-2-(2,6-dichlorophenyl)-5-[(2,4-difluorobenzyl)oxy]pyridazin-3(2H)-one;
4-bromo-2-(2,6-dichlorophenyl)-5-[(3,4-difluorobenzyl)oxy]pyridazin-3(2H)-one;
2-(2,6-dichlorophenyl)-5-(2-phenylethoxy)pyridazin-3(2H)-one;
2-(2,6-dichlorophenyl)-4-methyl-5-(2-phenylethoxy)pyridazin-3(2H)-one;
2-(2,6-dichlorophenyl)-4-methoxy-5-(2-phenylethoxy)pyridazin-3(2H)-one;
2-(2,6-dichlorophenyl)-4-isobutyl-5-(2-phenylethoxy)pyridazin-3(2H)-one;
2-(2,6-dichlorophenyl)-4-phenoxy-5-(2-phenylethoxy)pyridazin-3(2H)-one;
4-bromo-5-(2-phenylethoxy)pyridazin-3(2H)-one;
4-bromo-5-(2-phenylethoxy)-2-(pyridin-4-ylmethyl)pyridazin-3(2H)-one;
4-bromo-2-(2-hydroxyethyl)-5-(2-phenylethoxy)pyridazin-3(2H)-one;
4-bromo-5-[(2,4-difluorobenzyl)oxy]pyridazin-3(2H)-one;
4-bromo-5-[(2,4-difluorobenzyl)oxy]-2-(pyridin-4-ylmethyl)pyridazin-3(2H)-one;
4-bromo-5-[(2,4-difluorobenzyl)oxy]-2-[2-(dimethylamino)ethyl]pyridazin-3(2H)-one;
4-bromo-5-[(2,4-difluorobenzyl)oxy]-2-[3-(dimethylamino)propyl]pyridazin-3(2H)-one;
4-bromo-5-[(2,4-difluorobenzyl)oxy]-2-(2-hydroxyethyl)pyridazin-3(2H)-one; or
4-bromo-5-[(2,4-difluorobenzyl)oxy]-2-[(2-methyl-1,3-thiazol-4-yl)methyl]pyridazin-3(2H)-one;
and the pharmaceutically acceptable salts thereof.

### EMBODIMENT 57

A compound of the formula 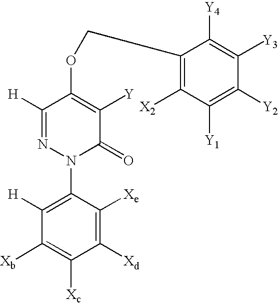
or pharmaceutically acceptable salts thereof, wherein
X₂, X_{b}, X_{c}, X_{d}, and Xₑ are independently selected from —C(O)NR₆R₇, —NR₆R₇, hydroxy(C₁-C₄)alkyl, C₁-C₄ dihydroxyalkyl, H, OH, halogen, haloalkyl, alkyl, alkoxy, haloalkoxy, heteroaryl, heterocycloalkyl, C₃-C₇ cycloalkyl, R₆R₇N—(C₁-C₆ alkyl)-, —CO₂—(C₁-C₆)alkyl, —N(R)C(O)NR₆R₇, —N(R)C(O)—(C₁-C₆)alkoxy, CO₂R-(C₁-C₆ alkyl)-, or —SO₂NR₆R₇; wherein the heteroaryl and heterocycloalkyl groups are optionally substituted with —NR₆R₇, —C(O)NR₆R₇, R₆R₇N—(C₁-C₆ alkyl)-, C₁-C₆ alkyl, C₁-C₆ alkoxy, or halogen;
   R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₄ dihydroxyalkyl, C₁-C₆ thiohydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, SH, NH₂, NH(alkyl), N(alkyl)(alkyl), —O—C₁-C₄ alkanoyl, C₁-C₄ alkyl, CF₃, or OCF₃; or
   R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen;
   R at each occurrence is independently H or C₁-C₆ alkyl; and
Y, Y₁, Y₂, Y₃, and Y₄ are independently selected from H, halogen, alkyl, carboxaldehyde, hydroxyalkyl, dihydroxyalkyl, alkenyl, alkynyl, CN, alkanoyl, alkoxy, alkoxyalkyl, haloalkyl, and carboxyl.

### EMBODIMENT 58

A compound according to embodiment 57, wherein
two Of X_{b}, X_{c}, and X_{d} are hydrogen and the other is —C(O)NR₆R₇, —(C₁-C₆ alkyl)-C(O)NR₆R₇, —NR₆R₇, R₆R₇N—(C₁-C₆ alkyl)- or —CO₂—(C₁-C₆)alkyl; wherein
   R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, NH₂, NH(alkyl), N(alkyl)(alkyl), —O—C₁-C₄ alkanoyl, C₁-C₄ alkyl, CF₃, or OCF₃; or
   R₆, R₇, and the nitrogen to which they are attached form a morpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen; and
Xₑ is hydrogen, C₁-C₄ alkyl, C₁-C₄ alkoxy, or halogen.

### EMBODIMENT 59

A compound according to embodiment 58, wherein
X_{b} is —C(O)NR₆R₇, —(C₁-C₆ alkyl)-C(O)NR₆R₇, —NR₆R₇, or R₆R₇N—(C₁-C₆ alkyl)-wherein
   R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₁-C₆ alkoxy, OH, NH₂, NH(alkyl), N(alkyl)(alkyl), —O—C₁-C₄ alkanoyl, C₁-C₄ alkyl, CF₃, or OCF₃.

### EMBODIMENT 60

A compound according to embodiment 59, wherein
X_{b} is —NR₆R₇, or —C(O)NR₆R₇;
X_{c} and X_{d} are both hydrogen;
Y₁ is selected from H, halogen, alkyl, hydroxyalkyl, CN, alkanoyl, alkoxy, alkoxyalkyl, CF₃, and carboxyl; and
Y₂, and Y₄ are independently selected from H, halogen, C₁-C₄ alkyl, C₁-C₄ hydroxyalkyl, C₁-C₄ alkoxy, C₁-C₄ alkoxy C₁-C₄ alkyl, and CF₃.

### EMBODIMENT 61

A compound according to embodiment 60, wherein
Xₑ is methyl, ethyl, C₁-C₃ alkoxy, or halogen;
Y is methyl or halogen.

### EMBODIMENT 62

A compound according to embodiment 61, wherein
Y₃ is H, halogen, or C₁-C₄ alkyl;
Y₂ and Y₄ are independently H or halogen;
X₂ is selected from H, halogen, R₆R₇N—(C₁-C₆ alkyl)-, —N(R)C(O)NR₆R₇, or —N(R)C(O)—(C₁-C₆)alkoxy;
R₆ is hydrogen or C₁-C₄ alkyl; and
R₇ is H, OH, C₁-C₆ alky, alkoxy, or C₁-C₆ alkanoyl, wherein the alkyl, alkoxy, and alkanoyl groups are optionally substituted with 1, 2, or 3 groups that are independently NH₂, NH(C₁-C₆ alkyl), N(C₁-C₆ alkyl)(C₁-C₆ alkyl), C₃-C₆ cycloalkyl, OH, or C₁-C₄ alkoxy.

### EMBODIMENT 63

A compound according to embodiment 62, wherein
Xₑ is halogen or methyl;
Y is methyl, chloro or bromo;
Y₁ is selected from H, halogen, C₁-C₄ alkyl, C₁-C₄ alkoxy, C₁-C₄ alkoxy C₁-C₄ alkyl, and CF₃;
R₇ is H, C₁-C₆ alkyl, C₁-C₆ alkoxy, or C₁-C₆ alkanoyl wherein each of the above is optionally substituted with 1, or 2 groups that are independently OH, SH, or halogen.

### EMBODIMENT 64

A compound according to embodiment 63, wherein
X₂ is selected from H, halogen, or R₆R₇N—(C₁-C₆ alkyl)-;
R₇ is H, C₁-C₆ alkyl, or C₁-C₆ alkanoyl each of the above is optionally substituted with 1, or 2 groups that are independently OH, SH, or halogen.

### EMBODIMENT 65

A compound according to embodiment 57, wherein
X_{b} is —NR₆R₇, or —C(O)NR₆R₇;
X_{c} and X_{d} are simultaneously H;
Xₑ is methyl or methoxy;
Y is methyl, Cl or Br;
Y₁ and Y₃ are simultaneously H;
Y₂ and Y₄ are independently H, F, Cl, Me, OMe, —CH₂OH, —CH₂— OCH₃;
X₂ is selected from H, halogen, R₆R₇N—(C₁-C₆ alkyl)-, —N(R)C(O)NR₆R₇, or —N(R)C(O)—(C₁-C₆)alkoxy;
R₆ is hydrogen or C₁-₄ alkyl; and
R₇ is H, OH, C₁-C₆ alky, alkoxy, or C₁-C₆ alkanoyl, wherein the alkyl, alkoxy, and alkanoyl groups are optionally substituted with 1, 2, or 3 groups that are independently NH₂, NH(C₁-C₆ alkyl), N(C₁-C₆ alkyl)(C₁-C₆ alkyl), C₃-C₆ cycloalkyl, OH, or C₁-₄ alkoxy.

### EMBODIMENT 66

A compound according to embodiment 65, wherein
X_{b} is-C(O)NR₆R₇;
Xₑ is methyl;
Y₂ and Y₄ are independently H, F, Me, or OMe; and
X₂ is H, halogen, R₆R₇N—(C₁-C₆ alkyl)-, or —N(R)C(O)—(C₁-C₆)alkoxy.

### EMBODIMENT 67

A compound according to embodiment 66, wherein
R₆ and R₇ are independently H or alkyl optionally substituted with 1 or 2 groups that are independently NH₂, NH(C₁-C₆ alkyl), N(C₁-C₆ alkyl)(C₁-C₆ alkyl), C₃-C₆ cycloalkyl, OH, or C₁-C₄ alkoxy.

### EMBODIMENT 68

A compound according to embodiment 66, wherein
R₆ and R₇ are independently H or alkyl optionally substituted with 1 group that is selected from NH₂, NH(C₁-C₄ alkyl), N(C₁-C₄ alkyl)(C₁-C₄ alkyl), OH, or C₁-C₄ alkoxy.

### EMBODIMENT 69

A compound according to embodiment 66, wherein
R₆ and R₇ are both H.

### EMBODIMENT 70

A compound according to embodiment 66, wherein
Y₂ and Y₄ are independently F, Me, or OMe.

### EMBODIMENT 71

A compound according to embodiment 70, wherein
R₆ and R₇ are independently H or C₁-C₆ alkyl.

### EMBODIMENT 72

A compound according to embodiment 71, wherein
X₂ is H.

### EMBODIMENT 73

A compound according to embodiment 72, wherein
at least one of R₆ and R₇ is H.

### EMBODIMENT 74

A compound according to embodiment 73, wherein
Y₂ and Y₄ are simultaneously fluorine.

The invention further comprises a pharmaceutical composition for the treatment of a TNF mediated disorder, a p38 kinase mediated disorder, inflammation, and/or arthritis, comprising a therapeutically-effective amount of a compound of Formula I, or a therapeutically-acceptable salt or tautomer thereof, in association with at least one pharmaceutically-acceptable carrier, adjuvant, solvent, excipient, or diluent.

The invention also comprises a therapeutic method of treating a TNF mediated disorder, a p38 kinase-alpha mediated disorder, inflammation and/or arthritis in a subject, the method comprising treating a subject having or susceptible to such disorder or condition with a therapeutically-effective amount of at least one compound of Formula I.

A preferred disorder treated according to the methods of the invention is a p38 kinase-alpha mediated disorder.

Specific diseases or conditions that can be treated using compounds of Formula I include:
inflammation;
arthritis, including but not limited to, rheumatoid arthritis, spondylarthropathies, gouty arthritis, gouty arthritis, osteoarthritis, systemic lupus erthematosus and juvenile arthritis, osteoarthritis, gouty arthritis and other arthritic conditions;
neuroinflammation;
pain (i.e., use as an analgesic) including but not limited to neuropathic pain;
fever (i.e., use as an antipyretic);
pulmonary disorders or lung inflammation, including adult respiratory distress syndrome, pulmonary sarcoisosis, asthma, silicosis, and chronic pulmonary inflammatory disease;
cardiovascular diseases including arteriosclerosis, myocardial infarction, thrombosis, congestive heart failure, and cardiac reperfusion injury;
cardiomyopathy;
reperfusion injury;
renal reperfusion injury;
ischemia including stroke and brain ischemia;
brain trauma;
brain edema;
liver disease and nephritis;
gastrointestinal conditions such as inflammatory bowel disease, Crohn's disease, gastritis, irritable bowel syndrome and ulcerative colitis;
ulcerative diseases such as gastric ulcer;
ophthalmic diseases such as retinitis, retinopathies, uveitis, ocular photophobia, and of acute injury to the eye tissue;
ophthalmological conditions such as corneal graft rejection, ocular neovascularization, retinal neovascularization including neovascularization following injury or infection, diabetic retinopathy, retrolental fibroplasias and neovascular glaucoma;
diabetes;
diabetic nephropathy;
skin-related conditions such as psoriasis, eczema, burns, dermatitis, keloid formation, scar tissue formation, and angiogenic disorders;
viral and bacterial infections, including sepsis, septic shock, gram negative sepsis, malaria, meningitis, opportunistic infections, cachexia secondary to infection or malignancy, cachexia secondary to acquired immune deficiency syndrome (AIDS), AIDS, ARC (AIDS related complex), pneumonia, and herpes virus;
myalgias due to infection;
influenza;
endotoxic shock;
toxic shock syndrome;
autoimmune disease including graft vs. host reaction and allograft rejections;
treatment of bone resorption diseases, such as osteoporosis;
multiple sclerosis;
disorders of the female reproductive system such as endometriosis;
pathological, but non-malignant, conditions such as hemaginomas, including infantile hemagionmas, angiofibroma of the nasopharynz and avascular necrosis of bone;
benign and malignant tumors/neoplasia including cancer, such as colorectal cancer, brain cancer, bone cancer, epithelial call-derived neoplasia (epithelial carcinoma) such as basal cell carcinoma, adenocarcinoma, gastrointestinal cancer such as lip cancer, mouth cancer, esophageal cancer, small bowel cancer and stomach cancer, colon cancer, liver cancer, bladder cancer, pancreas cancer, ovarian cancer, cervical cancer, lung cancer, breast cancer and skin cancer, such as squamous cell and basal cell cancers, prostate cancer, renal cell carcinoma, and other known cancers that affect epithelial cells throughout the body;
leukemia;
lymphoma;
systemic lupus erthrematosis (SLE);
angiogenesis including neoplasia;
metastasis; and
central nervous system disorders (including, but not limited to, central nervous system disorders having an inflammatory or apoptotic component), such as Alzheimer's disease, Parkinson's disease, Huntington's disease, amyotrophic lateral sclerosis, spinal cord injury, and peripheral neuropathy.

Compounds of formula I are preferably directed at treating inflammatory disorders.

The invention also provides a method of treating a p38 kinase or TNF mediated disorder comprising administering to a patient in need thereof a therapeutically effective amount of a compound according to claim 1 and at least one pharmaceutically acceptable carrier, adjuvant, solvent or excipient.

Definitions

As used herein, the term “alkenyl” refers to a straight or branched hydrocarbon of a designed number of carbon atoms containing at least one carbon-carbon double bond. Examples of “alkenyl” include vinyl, allyl, and 2-methyl-3-heptene.

The term “alkoxy” represents an alkyl attached to the parent molecular moiety through an oxygen bridge. Examples of alkoxy groups include, for example, methoxy, ethoxy, propoxy and isopropoxy.

The term “thioalkoxy” represents an alkyl attached to the parent molecular moiety through a sulfur atom. Examples of thioalkoxy groups include, for example, thiomethoxy, thioethoxy, thiopropoxy and thioisopropoxy.

As used herein, the term “alkyl” includes those alkyl groups of a designated number of carbon atoms. Alkyl groups may be straight or branched. Examples of “alkyl” include methyl, ethyl, propyl, isopropyl, butyl, iso-, sec- and tert-butyl, pentyl, hexyl, heptyl, 3-ethylbutyl, and the like. “Cx-Cy alkyl” represents an alkyl group of the specified number of carbons. For example, C₁-C₄ alkyl includes all alkyl groups that include at least one and no more than four carbon atoms. It also contains subgroups, such as, for example, C₂-C₃ alkyl or C₁-C₃ alkyl.

The term “aryl” refers to an aromatic hydrocarbon ring system containing at least one aromatic ring. The aromatic ring may optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl. The most preferred aryl group is phenyl. Aryl rings can be unsubstituted or can optionally carry substituents as indicated above.

The term “arylalkyl” refers to an aryl group, as defined above, attached to the parent molecular moiety through an alkyl group, as defined above. Preferred arylalkyl groups include, benzyl, phenethyl, phenpropyl, and phenbutyl. The more preferred arylalkyl groups include benzyl and phenethyl.

The term “arylalkoxy” refers to an aryl group, as defined above, attached to the parent molecular moiety through an alkoxy group, as defined above. Preferred arylalkoxy groups include, benzyloxy, phenethyloxy, phenpropyloxy, and phenbutyloxy. The more preferred arylalkoxy groups are benzyloxy and phenethyloxy. Most preferred is benzyloxy.

The term “cycloalkyl” refers to a C₃-C₈ cyclic hydrocarbon. Examples of cycloalkyl include cyclopropyl, cyclobutyl, cyclopentyl, cyclohexyl, cycloheptyl and cyclooctyl. Preferred cycloalkyl groups include cyclopropyl. Cycloalkyl groups can be unsubstituted or can optionally carry substituents as indicated above.

The term “cycloalkylalkyl,” as used herein, refers to a C₃-C₈ cycloalkyl group attached to the parent molecular moiety through an alkyl group, as defined above. Examples of cycloalkylalkyl groups include cyclopropylmethyl and cyclopentylethyl.

The terms “halogen” or “halo” indicate fluorine, chlorine, bromine, and iodine.

The term “heterocycloalkyl,” refers to a non-aromatic ring system containing at least one heteroatom selected from nitrogen, oxygen, and sulfur, wherein the non-aromatic heterocycle is attached to the core. The heterocycloalkyl ring may be optionally fused to or otherwise attached to other heterocycloalkyl rings, aromatic heterocycles, aromatic hydrocarbons and/or non-aromatic hydrocarbon rings. Preferred heterocycloalkyl groups have from 3 to 7 members. Examples of heterocycloalkyl groups include, for example, piperazine, 1,2,3,4-tetrahydroisoquinoline, morpholine, piperidine, tetrahydrofuran, pyrrolidine, and pyrazole. Preferred heterocycloalkyl groups include piperidinyl, piperazinyl, morpholinyl, and pyrolidinyl. Heterocycloalkyl groups can be unsubstituted or can optionally carry substituents as indicated above.

The term “heteroaryl” refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl. More preferred heteroaryl groups include pyridyl and thiazolyl. Heteroaryl groups can be unsubstituted or can optionally carry substituents as indicated above.

As used herein, the term “p38 mediated disorder” refers to any and all disorders and disease states in which p38 plays a role, either by control of p38 itself, or by p38 causing another factor to be released, such as but not limited to IL-1, IL-6 or IL-8. A disease state in which, for instance, IL-1 is a major component, and whose production or action, is exacerbated or secreted in response to p38, would therefore be considered a disorder mediated by p38.

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

As TNF-beta has close structural homology with TNF-alpha (also known as cachectin), and since each induces similar biologic responses and binds to the same cellular receptor, the synthesis of both TNF-alpha and TNF-beta are inhibited by the compounds of the invention and thus are herein referred to collectively as “TNF” unless specifically delineated otherwise.

Non-toxic pharmaceutically acceptable salts include, but are not limited to salts of inorganic acids such as hydrochloric, sulfuric, phosphoric, diphosphoric, hydrobromic, and nitric or salts of organic acids such as formic, citric, malic, maleic, fumaric, tartaric, succinic, acetic, lactic, methanesulfonic, p-toluenesulfonic, 2-hydroxyethylsulfonic, salicylic and stearic. Similarly, pharmaceutically acceptable cations include, but are not limited to sodium, potassium, calcium, aluminum, lithium and ammonium. Those skilled in the art will recognize a wide variety of non-toxic pharmaceutically acceptable addition salts. The invention also encompasses prodrugs of the compounds of Formula I.

The invention also encompasses the acylated prodrugs of the compounds of Formula I. Those skilled in the art will recognize various synthetic methodologies, which may be employed to prepare non-toxic pharmaceutically acceptable addition salts and acylated prodrugs of the compounds encompassed by Formula I.

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

The invention also encompasses the prodrugs of the compounds of Formula I. Those skilled in the art will recognize various synthetic methodologies that may be employed to prepare non-toxic pharmaceutically acceptable prodrugs of the compounds encompassed by Formula I. Those skilled in the art will recognize a wide variety of non-toxic pharmaceutically acceptable solvates, such as water, ethanol, mineral oil, vegetable oil, and dimethylsulfoxide.

The compounds of general Formula I may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants, and if desired other active ingredients. The pharmaceutical compositions containing compounds of general Formula, I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of general Formula I may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For disorders of the eye or other external tissues, e.g., mouth and skin, the formulations are preferably applied as a topical gel, spray, ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or oil or with both a fat and an oil. Preferably, a hydrophilic emulsifier is included together with a lipophilic emulsifier, which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base, which forms the oily, dispersed phase of the cream formulations. Emulsifiers and emulsion stabilizers suitable for use in the formulation of the invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The anti-inflammatory active ingredients are preferably present in such formulations in a concentration of 0.5 to 20%, advantageously 0.5 to 10% and particularly about 1.5% w/w. For therapeutic purposes, the active compounds of this combination invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water.

The disclosures in this application of all articles and references, including patents, are incorporated herein by reference.

The invention is illustrated further by the following examples, which are not to be construed as limiting the invention in scope or spirit to the specific procedures described in them.

The starting materials and various intermediates may be obtained from commercial sources, prepared from commercially available compounds, or prepared using well-known synthetic methods.

General Synthetic Procedures

The compounds of the invention can be prepared using methods well known in the art of organic synthesis. Representative procedures for preparing compounds of the invention are outlined in the following schemes.

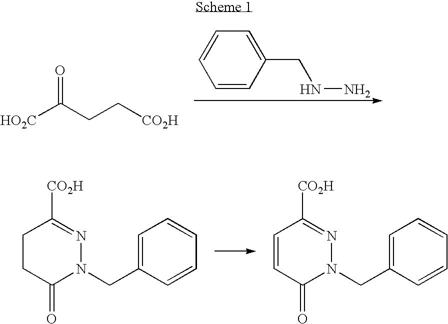

Various methods can be used for preparing the compounds of the invention. Examples of methods of preparing the compounds of the invention include the following. Compounds of the invention can be prepared by reacting a mono keto diacid with an appropriately substituted hydrazine to form a cyclized, partially saturated structure, as shown in Scheme 1. This structure is oxidized to the 6-carboxylic acid pyridazinone through methods well known in the art. The 6-carboxylic acid pyridazinone is further elaborated using methods well known in the art of organic chemistry and medicinal chemistry. For example, the carboxylic acid group is reduced to an alcohol and then converted into an ether or into a halide. Or the carboxylic acid group is converted into an amide or ester.

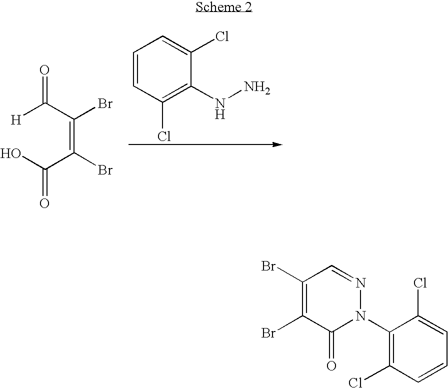

The compounds of the invention can be prepared by reacting the dibromo compound with an appropriately substituted hydrazine to form the 4,5 dibromopyridazinone. The 4,5 dibromopyridazinone is further manipulated as shown in schemes 3, 4, and 5, or it is subjected to organometallic coupling reactions such as the Heck reaction, Suzuki coupling, or Stille, coupling.

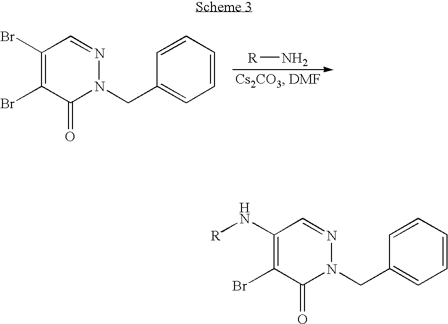

The 4,5 dibromopyridazinone prepared as in scheme 2 is converted into a 4-bromo 3-amino pyridazinone using methods well known in the art of organic synthesis and medicinal chemistry. Such methods include reacting the pyridazinone with a nucleophile in the presence of a sterically hindered base. R is aryl, heteroaryl, heterocycloalkyl, alkyl, arylalkyl, heteroarylalkyl or heterocycloalkyl groups. The resulting amine is further manipulated, for example, to generate amides, imides, or tertiary amines.

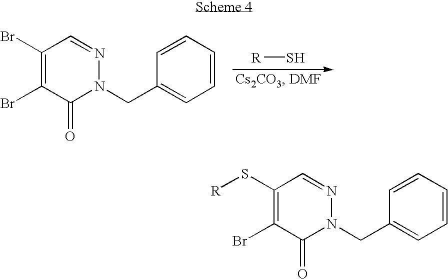

The 4,5 dibromopyridazinone prepared as in scheme 2 is converted into 5 thio pyridazinones using methods well known in the art of organic synthesis and medicinal chemistry. Such methods include reacting the pyridazinone with a nucleophile in the presence of a sterically hindered base. R is aryl, heteroaryl, heterocycloalkyl, alkyl, arylalkyl, heteroarylalkyl or heterocycloalkyl groups. Once the thioether compound has been made, it is further manipulated to generate the sulfoxide or the sulfone.

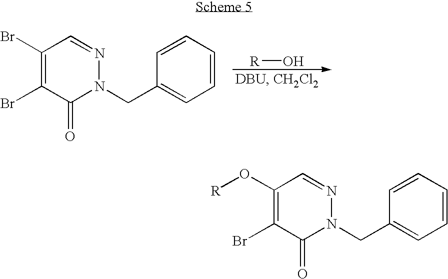

The 4,5 dibromopyridazinone prepared as in scheme 2 can also be converted into 5 alkoxy pyridazinones using methods well known in the art of organic synthesis and medicinal chemistry. Such methods include reacting the pyridazinone with a nucleophile in the presence of a sterically hindered base. Other methods include treating the pyridazinone with a nucleophile in the presence of a an inorganic base such as CSCO₃, and R is aryl, heteroaryl, heterocycloalkyl, alkyl, arylalkyl, heteroarylalkyl or heterocycloalkyl groups.

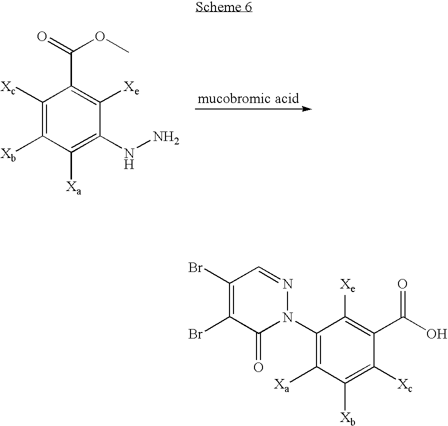

Further to scheme 1, scheme 6 illustrates the preparation of a pyridazinone core using a differently substituted hydrazine. The 4,5 dibromopyridazinone is further manipulated as shown in schemes 3, 4, and 5, or it is subjected to organometallic coupling reactions such as the Heck reaction, Suzuki coupling, or Stille, coupling. Or the carboxyl moiety may be manipulated to form an amide, or ester using methods well known in the art.

The invention is illustrated further by the following examples, which are not to be construed as limiting the invention in scope or spirit to the specific procedures described in them. Those having skill in the art will recognize that the starting materials may be varied and additional steps employed to produce compounds encompassed by the invention, as demonstrated by the following examples. Those skilled in the art will also recognize that it may be necessary to utilize different solvents or reagents to achieve some of the above transformations. In some cases, protection of reactive functionalities may be necessary to achieve the desired transformations. In general, such need for protecting groups, as well as the conditions necessary to attach and remove such groups, will be apparent to those skilled in the art of organic synthesis. When a protecting group is employed, deprotection step may be required. Suitable protecting groups and methodology for protection and deprotection such as those described in Protecting Groups in Organic Synthesis by T. Greene are well known and appreciated in the art.

Unless otherwise specified, all reagents and solvents are of standard commercial grade and are used without further purification. The appropriate atmosphere to run the reaction under, for example, air, nitrogen, hydrogen, argon and the like, will be apparent to those skilled in the art.

Experimental Section

### EXAMPLE 1

### 2-benzyl-4,5-dibromopyridazin-3(2H)-one

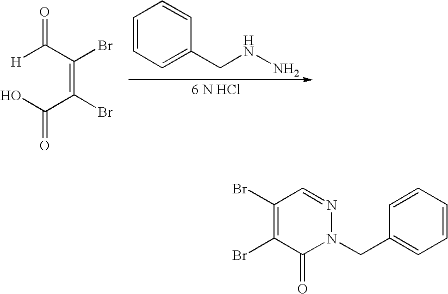

Mucobromic acid (10.0 g, 38.8 mmol) was dissolved in 300 ml of 6N HCl in a 500 ml round bottom flask at room temperature. Benzyl hydrazine di-hydrochloride (9.08 g, 46.5 mmol) was added and the reaction was stirred at room temperature. Both reagents quickly dissolved. After 30 minutes, the solution started becoming cloudy. The reaction was allowed to stir at room temperature for 18 hours. A large quantity of precipitate had formed, but LC/MS showed both starting materials still remained. The reaction was allowed to stir for another 18 hours. LC/MS showed most of the starting materials consumed. The reaction was extracted with ethyl acetate (3×100 ml). The combined organic layer was washed with 1 N HCl (2×100 ml), 1 N NaOH (2×100 ml) and brine (1×250 ml), dried over anhydrous MgSO₄ and filtered. The solvent was removed and the resulting white solid was dried under vacuum to afford 8.50 g of a white solid. ¹H NMR (300 MHz, CDCl₃) δ 7.82 (s, 1H), 7.48-7.32 (m, 5H), 5.33 (s, 2H); LC/MS, tᵣ=2.53 minutes (5 to 95% acetonitrile/water over 5 minutes at 1 ml/min, at 254 nm, at 50° C.), (M+H), Calculated=343, Found=343; HR/MS (M+H), Calculated=342.9076, Found=342.9089 (Δ mmu=1.3).

### EXAMPLE 2

### 4,5-dibromo-2-(2,6-dichlorophenyl)pyridazin-3(2H)-one

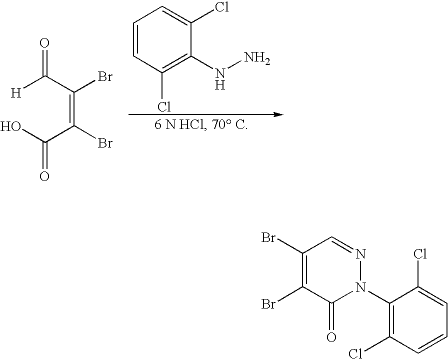

Mucobromic acid (50.0 g, 194 mmol) was dissolved in 1 L of 6N HCl in a 3 L three-necked round bottom flask at room temperature. 2,6-Dichlorophenyl hydrazine hydrochloride (49.7 g, 232.8 mmol) was added as a partial suspension in 500 ml of warm 6 N HCl. The reaction was stirred vigorously with a mechanical stirrer at 70° C. The heating aided in dissolving more of the hydrazine, however the reaction never totally went into solution. After 18 hours, LC/MS showed reaction completion. The reaction was allowed to partially cool. 1 L of ethyl acetate was then added in an attempt to extract the product. The precipitate went into solution, but the solution was homogenous and not two layers as expected. The reaction was allowed to stand in an attempt to allow the two layers to separate. As the reaction cooled, a large amount of precipitate formed. It was found that the HCl converted the ethyl acetate to ethanol and acetic acid, which caused the solution to become homogenous and caused product precipitation. The solid was filtered, washed with 1 L of diethyl ether and dried under vacuum to afford 66.1 g of an off-white solid. ¹H NMR (300 MHz, CDCl₃) δ 8.01 (s, 1H), 7.52-7.38 (m, 3H); LC/MS, tᵣ=2.76 minutes (5 to 95% acetonitrile/water over 5 minutes at 1 ml/min, at 254 nm, at 50° C.), (M+H), Calculated=397, Found=397; HR/MS (M+H), Calculated=396.8140, Found=396.8135 (Δ mmu=−0.5).

### EXAMPLE 3

### 4,5-dibromo-2-phenylpyridazin-3(2H)-one

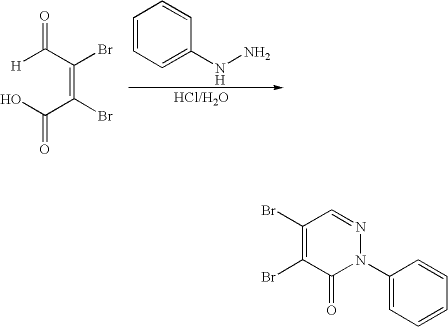

Mucobromic acid (10.0 g, 38.8 mmol) was dissolved in 250 ml of 6N HCl in a 500 ml round bottom flask at room temperature. Phenyl hydrazine (4.58 ml, 46.6 mmol) was dissolved in 100 ml of 6 N HCl and added to the reaction with vigorous stirring at 70° C. for 18 hours. An off-white precipitate formed immediately. After 18 hours, LC/MS showed reaction completion. The reaction was allowed to partially cool. 100 ml of ethyl acetate was then added in an attempt to extract the product. The precipitate went into solution, but the solution was homogenous and not two layers as expected. The reaction was allowed to stand in an attempt to allow the two layers to separate. As the reaction cooled, a large amount of precipitate formed. It was found that the HCl converted the ethyl acetate to ethanol and acetic acid, which caused the solution to become homogenous and caused product precipitation. The solid was filtered, washed with diethyl ether and dried under vacuum to afford 10.54 g of an off-white solid. ¹H NMR (300 MHz, CDCl₃) δ 7.96 (s, 1H), 7.60-7.42 (m, 5H); LC/MS, tᵣ=2.35 minutes (5 to 95% acetonitrile/water over 5 minutes at 1 ml/min, at 254 nm, at 50° C.), (M+H), Calculated=329, Found=329; HR/MS (M+H), Calculated=328.8920, Found=328.8927 (Δ mmu=0.7).

### EXAMPLE 4

### 2-benzyl-4-bromo-5-[(4-fluorobenzyl)oxy]pyridazin-3(2H)-one

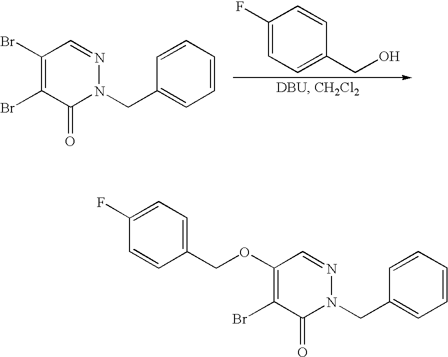

2-benzyl-4,5-dibromopyridazin-3(2H)-one (500 mg, 1.45 mmol) was dissolved in 5 ml of CH₂Cl₂ in a 15 ml round bottom flask at room temperature. 4-Fluorobenzyl alcohol (175 μl, 1.60 mmol) and DBU (433.7 μl, 2.9 mmol) were added and the reaction was stirred at room temperature for 18 hours. The reaction was diluted with 20 ml of CH₂Cl₂ and washed with 1 N HCl (2×10 ml), saturated NaHCO₃ (2×10 ml) and brine (2×10 ml). The organic layer was dried over anhydrous MgSO₄, filtered and evaporated to afford a tan solid. The solid was washed with ethyl acetate (2×5 ml) to remove some small impurities. Some of the product was lost, but the remaining solid was shown to be pure by LC/MS. The remaining solid was dried under vacuum to afford 264.2 mg of an off-white solid.

¹H NMR (300 MHz, CDCl₃) δ 7.70 (s, 1H), 7.46-7.30 (m, 7H), 7.12 (t, J=8.66, 2H), 5.36 (s, 2H), 5.28 (s, 2H); LC/MS, tᵣ=2.94 minutes (5 to 95% acetonitrile/water over 5 minutes at 1 ml/min, at 254 nm, at 50° C.), (M+H), Calculated=389, Found=389; HR/MS (M+H), Calculated=389.0295, Found=389.0308 (Δ mmu=1.3).

### EXAMPLE 5

### 2-benzyl-5-(benzyloxy)-4-bromopyridazin-3(2H)-one

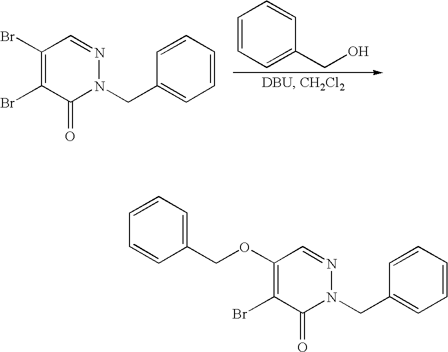

2-benzyl-4,5-dibromopyridazin-3(2H)-one (500 mg, 1.45 mmol) was dissolved in 5 ml of CH₂Cl₂ in a 15 ml round bottom flask at room temperature. Benzyl alcohol (166 μl, 1.60 mmol) and DBU (433.7 μl, 2.9 mmol) were added and the reaction was stirred at room temperature for 5 days. The reaction was diluted with 20 ml of CH₂Cl₂ and washed with 1 N HCl (2×10 ml), saturated NaHCO₃ (2×10 ml) and brine (2×10 ml). The organic layer was dried over anhydrous MgSO4, filtered, and evaporated to afford a tan solid. The solid was washed with diethyl ether and dried under vacuum to afford 335 mg of an off-white solid. ¹H NMR (300 MHz, CDCl₃) δ 7.72 (s, 1H), 7.46-7.30 (m, 10H), 5.35 (s, 2H), 5.33 (s, 2H); LC/MS, tᵣ=2.85 minutes (5 to 95% acetonitrile/water over 5 minutes at 1 ml/min, at 254 nm, at 50° C.), (M+H), Calculated=371, Found=371; HR/MS (M+H), Calculated=371.0390, Found=371.0380 (Δ mmu=−1.0).

### EXAMPLE 6

### 2-benzyl-5-(benzyloxy)pyridazin-3(2H)-one

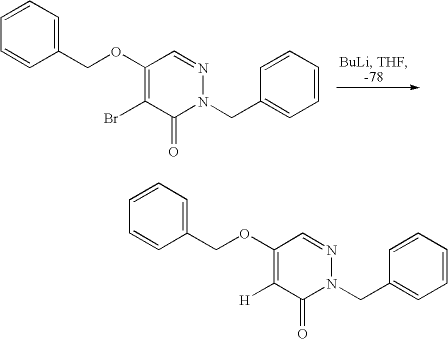

2-benzyl-5-(benzyloxy)-4-bromopyridazin-3(2H)-one (100 mg, 0.27 mmol) was dissolved in 4 ml of THF in a 15 ml round bottom flask at −78° C. n-BuLi (119 μl, 0.30 mmol) was added and the reaction was stirred at −78° C. for 5 minutes. The reaction was quenched with 5 ml of saturated NH₄Cl, extracted with ethyl acetate (1×15 ml) and dried over anhydrous Na₂SO₄, filtered and evaporated. The resulting oil was triturated with several solvents, but crystallization was unsuccessful.

¹H NMR (300 MHz, CDCd₃) δ 7.65 (d, J=2.82, 1H), 7.43-7.28 (m, 10H), 6.27 (d, J=2.62, 1H), 5.29 (s, 2H), 5.01 (s, 2H); LC/MS, tᵣ=2.70 minutes (5 to 95% acetonitrile/water over 5 minutes at 1 ml/min, at 254 nm, at 50° C.), (M+H), Calculated=293, Found=293.

### EXAMPLE 7

### 2-benzyl-4-bromo-5-[(4-fluorobenzyl)amino]pyridazin-3(2H)-one

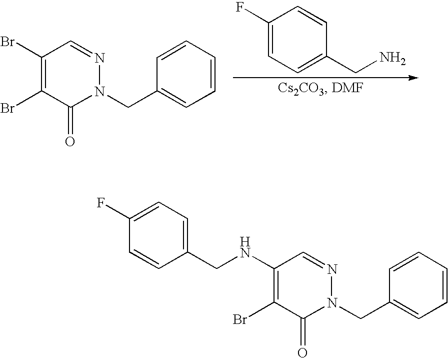

2-benzyl-4,5-dibromopyridazin-3(2H)-one (500 mg, 1.45 mmol) was dissolved in 5 ml of DMF in a 15 ml round bottom flask at room temperature. 4-Fluorobenzylamine (183 μl, 1.60 mmol,) and CsCO₃ (945 mg, 2.9 mmol) were added and the reaction was stirred vigorously at room temperature for 18 hours. The reaction was diluted with 50 ml of H₂O and extracted with ethyl acetate (3×50 ml). The combined organic layers were washed with 1 N HCl (2×100 ml), saturated NaHCO₃ (2×100 ml) and brine (2×100 ml). Attempts to precipitate the product failed, so silica gel flash chromatography was performed on a Biotage MPLC system (30% ethyl acetate in hexanes to 60% ethyl acetate in hexanes). The resulting solid was dried under vacuum to afford 164.5 mg of an off-white solid. ¹H NMR (300 MHz, CDCl₃) δ 7.46-7.26 (m, 8H), 7.09 (t, J=8.66, 2H), 5.31 (s, 2H), 4.50 (d, J=4.84, 2H); LC/MS, tᵣ=2.72 minutes (5 to 95% acetonitrile/water over 5 minutes at 1 ml/min, at 254 nm, at 50° C.), (M+H), Calculated=388, Found=388; HR/MS (M+H), Calculated=388.0455, Found=388.0433 (Δ mmu=−2.2).

### EXAMPLE 8

### 2-benzyl-4-bromo-5-[(tetrahydrofuran-2-ylmethyl)amino]pyridazin-3(2H)-one

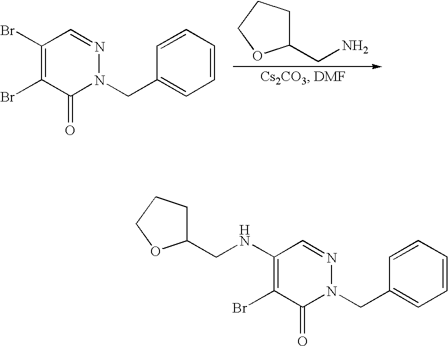

2-benzyl-4,5-dibromopyridazin-3(2H)-one (500 mg, 1.45 mmol) was dissolved in 5 ml of DMF in a 15 ml round bottom flask at room temperature. Tetrahydrofurfurylamine (165 μl, 1.60 mmol) and CsCO₃ (945 mg, 2.9 mmol) were added and the reaction was stirred vigorously at room temperature for 2 days. The reaction was diluted with 50 ml of H₂O and extracted with ethyl acetate (3×50 ml). The combined organic layers were washed with 1 N HCl (2×100 ml), saturated NaHCO₃ (2×100 ml) and brine (2×100 ml). The product was triturated with diethyl ether and the resulting solid was dried under vacuum to afford 154 mg of an off-white solid. ¹H NMR (300 MHz, CDCl₃) δ 7.56 (s, 1H), 7.45-7.29 (m, 5H), 5.33 (s, 2H), 5.11 (br s, 1H), 4.12 (m, 1H), 3.95-3.78 (m, 2H), 3.52-3.25 (m, 2H), 2.10-1.91 (m, 3H), 1.69-1.59 (m, 1H); LC/MS, tᵣ=2.27 minutes (5 to 95% acetonitrile/water over 5 minutes at 1 ml/min, at 254 nm, at 50° C.), (M+H), Calculated=364, Found=364; HR/MS (M+H), Calculated=364.0655, Found=364.0653 (Δ mmu=−0.2).

### EXAMPLE 9

### [(1-benzyl-5-bromo-6-oxo-1,6-dihydropyridazin-4-yl)amino](phenyl)acetic acid

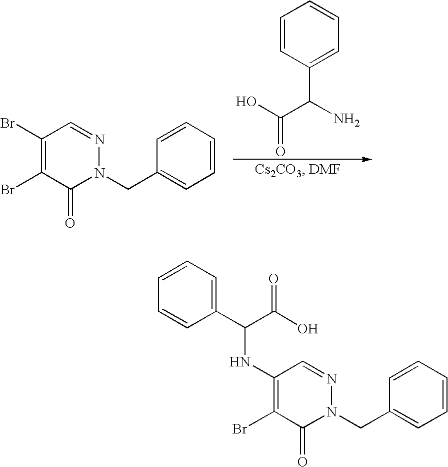

2-benzyl-4,5-dibromopyridazin-3(2H)-one (500 mg, 1.45 mmol) was dissolved in 5 ml of DMF in a 15 ml round bottom flask at room temperature. D,L-2-Phenylglycine (484 mg, 3.2 mmol) and CsCO₃ (1.56 g, 4.79 mmol) were added and the reaction was stirred vigorously at room temperature for 2 days. The reaction was diluted with 50 ml of H₂O and extracted with ethyl acetate (3×50 ml), which removed excess starting material. The aqueous layer was titrated to pH=7 with NH₄Cl and extracted with n-butanol (3×50 ml). The butanol layer was evaporated under vacuum and the resulting solid was washed with acetonitrile and dried under vacuum to afford 118 mg of a tan solid. ¹H NMR (300 MHz, CDCl₃) δ 7.45-7.26 (m, 11H), 6.36 (d, J=5.24, 1H), 5.36-5.20 (m, 4H); LC/MS, tᵣ=2.44 minutes (5 to 95% acetonitrile/water over 5 minutes at 1 ml/min, at 254 nm, at 50° C.), (M+H), Calculated=414, Found=414; HR/MS (M+H), Calculated=414.0448, Found=414.0461 (Δ mmu=1.3).

### EXAMPLE 10

### 2-benzyl-4-bromo-5-[(4-fluorophenyl)thio]pyridazin-3(2H)-one

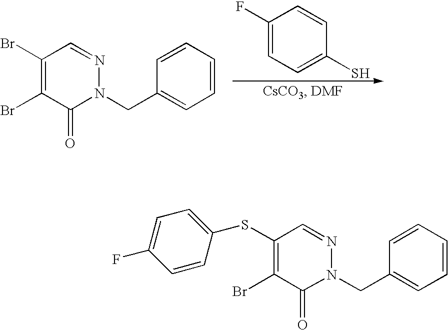

2-benzyl-4,5-dibromopyridazin-3(2H)-one (500 mg, 1.45 mmol) was dissolved in 5 ml of DMF in a 15 ml round bottom flask at room temperature. 4-Fluorothiophenol (156 μl, 1.46 mmol) and CsCO₃ (709 mg, 2.18 mmol) were added and the reaction was stirred vigorously at room temperature for 2.5 hours. The reaction was diluted with 50 ml of H₂O and extracted with ethyl acetate (3×50 ml). The combined organic layers were washed with 1 N HCl (2×100 ml), 1 N NaOH (2×100 ml) and brine (2×100 ml). The resulting oil was triturated with 25% ethyl acetate in hexanes. The resulting solid was dried under vacuum to afford 327 mg of an off-white solid. ¹H NMR (300 MHz, CDCl₃) δ 7.62-7.57 (m, 2H), 7.44-7.30 (m, 5H), 7.20 (t, J=8.46, 2H), 6.88 (s, 1H), 5.29 (s, 2H) ; LC/MS, tᵣ=3.32 minutes (5 to 95% acetonitrile/water over 5 minutes at 1 ml/min, at 254 nm, at 50° C.), (M+H), Calculated=391, Found=mmu=−1.6).

### EXAMPLE 11

### 2-benzyl-4-bromo-5-[(2-isopropylphenyl)thio]pyridazin-3(2H)-one

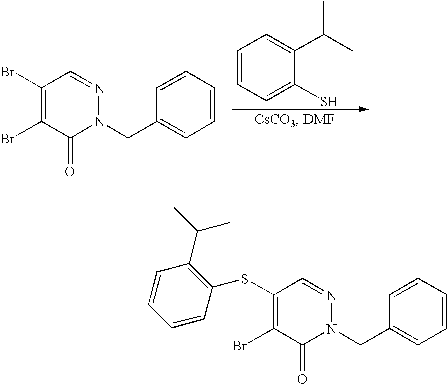

2-benzyl-4,5-dibromopyridazin-3(2H)-one (500 mg, 1.45 mmol) was dissolved in 5 ml of DMF in a 15 ml round bottom flask at room temperature. 2-Isopropylthiophenol (232 μl, 1.52 mmol) and CsCO₃ (709 mg, 2.18 mmol) were added and the reaction was stirred vigorously at room temperature for 18 hours. The reaction was diluted with 50 ml of H₂O and extracted with ethyl acetate (3×50 ml). The combined organic layers were washed with 1 N HCl (2×100 ml), 1 N NaOH (2×100 ml) and brine (2×100 ml). The resulting oil was triturated with diethyl ether. The resulting solid was dried under vacuum to afford 392 mg of an off-white solid. ¹H NMR (300 MHz, CDCl₃) δ 7.58-7.24 (m, 9H)₁ 6.80 (s, 1H), 5.28 (s, 2H), 3.56-3.43 (m, 1H), 1.23 (d, J=6.85, 6H); LC/MS, tᵣ=3.83 minutes (5 to 95% acetonitrile/water over 5 minutes at 1 ml/min, at 254 nm, at 50° C.), (M+H), Calculated=415, Found=415; HR/MS (M+H), Calculated=415.0474, Found=415.0495 (Δ mmu=2.1).

### EXAMPLE 12

### 4-bromo-2-(2,6-dichlorophenyl)-5-[(4-fluorobenzyl)oxy]pyridazin-3(2H)-one

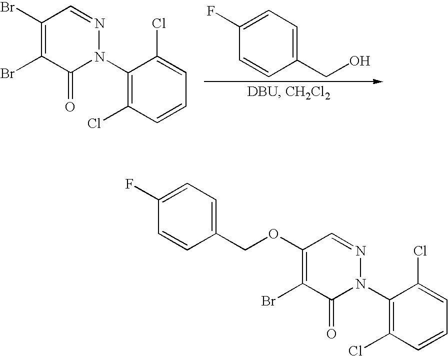

4,5-dibromo-2-(2,6-dichlorophenyl)pyridazin-3(2H)-one (500 mg, 1.25 mmol) was dissolved in 5 ml of CH₂Cl₂ in a 15 ml round bottom flask at room temperature. 4-Fluorobenzyl alcohol (150 μl, 1.38 mmol) and DBU (1,8-diazabicyclo[5.4.0]undec-7-ene) (374 μl, 2.5 mmol) were added and the reaction was stirred at room temperature for 18 hours. The reaction was diluted with 20 ml of CH₂Cl₂ and washed with 1 N HCl (2×10 ml), saturated NaHCO₃ (2×10 ml) and brine (2×10 ml). The organic layer was dried over anhydrous MgSO₄, filtered and evaporated to afford a tan solid. The solid was triturated with diethyl ether and dried under vacuum to afford 263 mg of an off-white solid. ¹H NMR (300 MHz, CDCl₃) δ 7.93 (s, 1H), 7.50-7.35 (m, 5H), 7.16 (m, 2H), 5.40 (s, 2H); LC/MS, tᵣ=3.04 minutes (5 to 95% acetonitrile/water over 5 minutes at 1 ml/min, at 254 nm, at 50° C.), (M+H), Calculated=443, Found=443; HR/MS (M+H), Calculated=442.9359, Found=442.9346 (Δ mmu=−1.3).

### EXAMPLE 13

### 4-bromo-2-(2,6-dichlorophenyl)-5-[(4-fluorobenzyl)amino]pyridazin-3(2H)-one

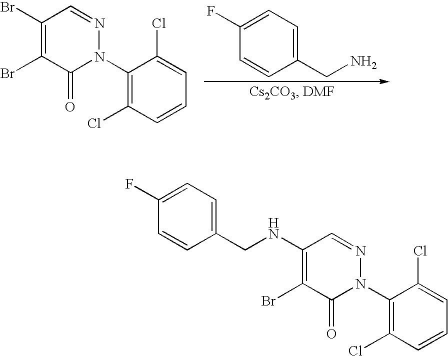

4,5-dibromo-2-(2,6-dichlorophenyl)pyridazin-3(2H)-one (500 mg, 1.25 mmol) was dissolved in 5 ml of DMF in a 15 ml round bottom flask at room temperature. 4-Fluorobenzylamine (157 μl, 1.38 mmol) and CsCO₃ (611 mg, 1.88 mmol) were added and the reaction was stirred vigorously at room temperature for 18 hours. The reaction was poured into 100 ml of H₂O, which caused the product to precipitate out. The resulting solid was triturated with diethyl ether and dried under vacuum to afford 254 mg of an off-white solid. ¹H NMR (300 MHz, CDCl₃) δ 7.68 (s, 1H), 7.48-7.30 (m, 5H), 7.14 (t, J=8.46, 2H), 5.39 (br s, 1H), 4.61 (d, J=5.44, 2H); LC/MS, tᵣ=2.74 minutes (5 to 95% acetonitrile/water over 5 minutes at 1 ml/min, at 254 nm, at 50° C.), (M+H), Calculated=442, Found=442; HR/MS (M+H), Calculated=441.9519, Found=441.9530 (Δ mmu=1.1).

### EXAMPLE 14

4-bromo-2-(2,6-dichlorophenyl)-5-[(4-fluorophenyl)thio]pyridazin-3(2H)-one

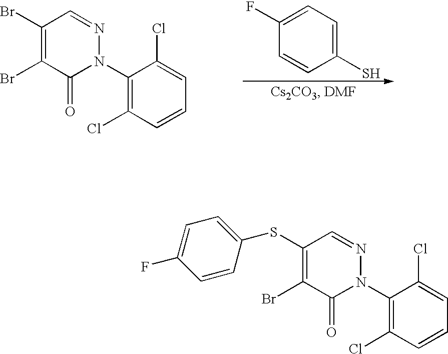

4,5-dibromo-2-(2,6-dichlorophenyl)pyridazin-3(2H)-one (500 mg, 1.25 mmol) was dissolved in 5 ml of DMF in a 15 ml round bottom flask at room temperature. 4-Fluorothiophenol (134 μl, 1.26 mmol) and CsCO₃ (611 mg, 1.88 mmol) were added and the reaction was stirred vigorously at room temperature for 1.5 hours. The reaction was poured into 100 ml of H₂O, which caused the product to precipitate out. The resulting solid was triturated with diethyl ether to give a denser, more granular solid than before. The resulting solid was dried under vacuum to afford 347 mg of an off-white solid. ¹H NMR (300 MHz, CDCl₃) δ 7.75-7.67 (m, 2H), 7.49-7.36 (m, 3H), 7.25 (t, J=8.46, 2H), 7.07 (s, 1H); LC/MS, tᵣ=3.41 minutes (5 to 95% acetonitrile/water over 5 minutes at 1 ml/min, at 254 nm, at 50° C.), (M+H), Calculated=445, Found=445; HR/MS (M+H), Calculated=444.8975, Found=444.8971 (Δ mmu=−0.4).

### EXAMPLE 15

### 3-[5-bromo-4-[(2,4-difluorobenzyl)oxy]-6-oxopyridazin-1(6H)-yl]-N,4-dimethylbenzamide

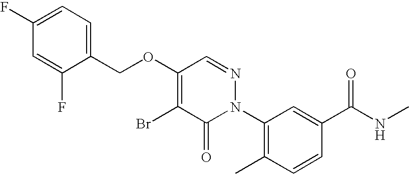

### Step 1: Preparation of methyl 3-hydrazino-4-methylbenzoate hydrochloride (PHA-828197A).

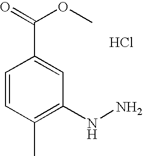

5-Carboxy-o-tolylhydrazine (10.0 g, 60.2 mmol) was dissolved in 80 ml methanol and treated with 120 ml of 4M HCl in 1,4-dioxane and stirred overnight at room temperature. The reaction was evaporated and the resulting solid was washed with acetone and dried in vacuo to give a beige solid (9.53 g, 73% yield). ¹H NMR (400 MHz, DMSO-d₆) δ 10.26 (br s, 3H), 8.07 (br s, 1H), 7.50-7.45 (m, 2H), 7.25 (d, J=7.65 Hz, 1H), 3.81 (s, 3H), 2.22 (s, 3H); LC/MS, tᵣ=0.62 minutes (5 to 95% acetonitrile/water over 5 minutes at 1 ml/min, at 254 nm, at 50° C.), ES-MS m/z 181 (M+H). ES-HRMS m/z 181.0999 (M+H calcd for C₉H₁₃N₂O₂requires 181.0972).

### Step 2: Preparation of 3-(4,5-dibromo-6-oxopyridazin-1(6H)-yl)-4-methylbenzoic acid

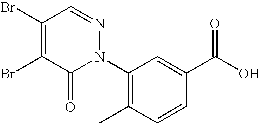

Mucobromic acid (9.74 g, 37.8 mmol) was dissolved in 150 ml of 6N HCl in a 500 ml round bottom flask at room temperature. Methyl 3-hydrazino-4-methylbenzoate hydrochloride (from Step 1 above) (9.0 g, 41.5 mmol) was dissolved in 150 ml of 6 N HCl and added to the reaction with vigorous stirring at 70° C. for 18 hours. An off-white precipitate formed immediately. The reaction was cooled and the resulting precipitate was filtered, washed with water and diethyl ether and dried in vacuo to give a tan solid (8.58 g, 58% yield). Spectra showed a 10% impurity of methylated product. ¹H NMR (400 MHz, DMSO-d₆) δ 13.06 (br s, 1H), 8.27 (s, 1H), 7.93-7.90 (m, 2H), 7.50 (d, J=7.93 Hz, 1H), 2.11 (s, 3H); LC/MS, tᵣ=2.14 minutes (5 to 95% acetonitrile/water over 5 minutes at 1 ml/min, at 254 nm, at 50° C.), ES-MS m/z 386.9 (M+H calcd for C₁₂H₉Br₂N₂O₃requires 386.9).

### Step 3: Preparation of methyl 3-(4,5-dibromo-6-oxopyridazin-1(6H)-yl)-4-methylbenzoate

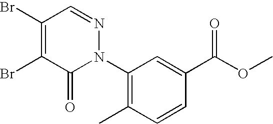

3-(4,5-dibromo-6-oxopyridazin-1(6H)-yl)-4-methylbenzoic acid (from Step 2) (8.3 g, 21.4 mmol) was dissolved as much as possible in 50 ml methanol. 50 ml of 4M HCl in 1,4-dioxane was added and stirred at room temperature overnight. The reaction was evaporated and the resulting solid was filtered, washed with diethyl ether and dried in vacuo to give a white solid (6.1 g, 71%). ¹H NMR (400 MHz, DMSO-d₆) δ 8.27 (s, 1H), 7.95-7.93 (m, 2H), 7.53 (d, J=8.46 Hz, 1H), 3.82 (s, 3H), 2.12 (s, 3H); LC/MS, tᵣ=2.37 minutes (5 to 95% acetonitrile/water over 5 minutes at 1 ml/min, at 254 nm, at 50° C.), ES-MS m/z 400 (M+H). ES-HRMS m/z 417.9386 (M+NH₄ calcd for C₁₃H₁₄Br₂N₃O₃requires 417.9396).

### Step 4: Preparation of methyl 3-[5-bromo-4-[(2,4-difluorobenzyl)oxy]-6-oxopyridazin-1(6H)-yl]-4-methylbenzoate

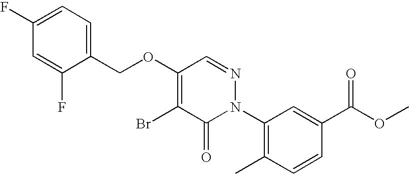

Methyl 3-(4,5-dibromo-6-oxopyridazin-1(6H)-yl)-4-methylbenzoate (from Step 3) (5.0 g, 12.4 mmol) was stirred briskly with 2,4-difluorobenzylalcohol (1.87 ml, 16.7 mmol) and Cs₂CO₃ (6.06 g, 18.6 mmol) in 50 ml of N,N-dimethylformamide at room temperature overnight. The reaction was then poured into 1L of cold water and the resulting precipitate was filtered, washed with water and diethyl ether, and dried in vacuo to yield a white solid (2.89 g, 50%). ¹H NMR (400 MHz, DMSO-d₆) δ 8.51 (s, 1H), 8.00 (dd, J=8.06, 1.61 Hz, 1H), 7.90 (d, J=1.61 Hz, 1H), 7.73 (app q, J=7.92 Hz, 1H), 7.58 (d, J=8.06 Hz, 1H), 7.41 (dt, J=9.97, 2.15 Hz, 1H), 7.23 (dt, J=8.56, 1.75 Hz, 1H), 5.59 (s, 2H), 3.88 (s, 3H), 2.15 (s, 3H); LC/MS, tᵣ=2.68 minutes (5 to 95% acetonitrile/water over 5 minutes at 1 ml/min, at 254 nm, at 50° C.), ES-MS m/z 465 (M+H). ES-HR/MS m/z 465.0274 (M+H calcd for C₂₀H₁₆BrF₂N₂O₄ requires 465.0256).

### Step 5: Preparation of the Title Compound

Methyl 3-[5-bromo-4-[(2,4-difluorobenzyl)oxy]-6-oxopyridazin-1(6H)-yl]-4-methylbenzoate (from Step 4 above) (2.5 g, 5.37 mmol) was stirred with 4.3 ml of 2.5 N NaOH in 10 ml tetrahydrofuran and 2 ml water overnight. The reaction was acidified with 1 N HCl and 25 ml of ethyl acetate was added. The resulting precipitate was filtered and discarded. The filtrate was extracted 2 times with ethyl acetate. The combined organic layer was washed 3 times with water, dried over MgSO₄ and evaporated. Diethyl ether was used to dissolve the product and triturate the impurities, which were filtered off. The filtrate was evaporated to give a crude oil. The oil (590 mg, 1.31 mmol) was dissolved in 5 ml of methylene chloride. 2.0 M methylamine in tetrahydrofuran (2.61 ml, 5.23 mmol) was added, followed, in order, by EDCI (314 mg, 1.64 mmol), 1-hydroxybenzotriazole (222 mg, 1.64 mmol) and triethylamine (365 μl, 2.62 mmol). The reaction was stirred at room temperature overnight. The reaction was quenched with ammonium chloride and extracted 2 times with ethyl acetate. The combined organic layer was washed with brine, dried over MgSO₄ and evaporated. The resulting oil was triturated with diethyl ether to obtain a solid, which was dried in vacuo to give a white solid (46 mg, 8%). ¹H NMR (400 MHz, DMSO-d₆) δ 8.47 (s, 1H), 8.00 (dd, J=8.06, 1.75 Hz, 1H), 7.72 (d, J=1.75 Hz, 1H), 7.68 (app q, J=7.52 Hz, 1H), 7.45 (d, J=7.92 Hz, 1H), 7.35 (dt, J=9.87, 2.55 Hz, 1H), 7.18 (dt, J=8.19, 2.06 Hz, 1H), 5.55 (s, 2H), 4.46 (d, J=3.59 Hz, 1H), 2.75 (d, J=4.43 Hz, 3H), 2.06 (s, 3H) ; LC/MS, tᵣ=2.29 minutes (5 to 95% acetonitrile/water over 5 minutes at 1 ml/min, at 254 nm, at 50° C.), ES-MS m/z 464 (M+H). ES-HR/MS m/z 481.0663 (M+NH₄ calcd for C₂₀H₂₀BrF₂N₄O₃ requires 481.0681).

Biological Evaluation

p38 Kinase Assay

Cloning of Human p38 Kinase-alpha:

The coding region of the human p38 Kinase-alpha cDNA was obtained by PCR-amplification from RNA isolated from the human monocyte cell line THP.1. First strand CDNA was synthesized from total RNA as follows: 2 μg of RNA was annealed to 100 ng of random hexamer primers in a 10 μl reaction by heating to 70° C. for 10 minutes followed by 2 minutes on ice. cDNA was then synthesized by adding 1 μl of RNAsin (Promega, Madison Wis.), 2 μl of 50 mM dNTP's, 4 μl of 5× buffer, 2 μl of 100 mM DTT and 1 μl (200 U) of Superscript II.™. AMV reverse transcriptase. Random primer, dNTP's and Superscript.™. reagents were all purchased from Life-Technologies, Gaithersburg, Mass. The reaction was incubated at 42° C. for 1 hour. Amplification of p38 cDNA was performed by aliquoting 5 μl of the reverse transcriptase reaction into a 100 μl PCR reaction containing the following: 80 μl dH.sub.2 O, 2. μl 50 mM dNTP's, 1 μl each of forward and reverse primers (50 pmol/μl), 10 μl of 10 × buffer and 1 μl Expand™. polymerase (Boehringer Mannheim). The PCR primers incorporated Bam HI sites onto the 5′ and 3′ end of the amplified fragment, and were purchased from Genosys. The sequences of the forward and reverse primers were 5′-GATCGAGGATTCATGTCTCAGGAGAGGCCCA-3′ and 5′GATCGAGGATTCTCAGGACTCCATCTCTTC-3′ respectively. The PCR amplification was carried out in a DNA Thermal Cycler (Perkin Elmer) by repeating 30 cycles of 940 C. for 1 minute, 60° C. for 1 minute and 68° C. for 2 minutes. After amplification, excess primers and unincorporated dNTP's were removed from the amplified fragment with a Wizard™. PCR prep (Promega) and digested with Bam HI (New England Biolabs). The Bam HI digested fragment was ligated into BamHI digested pGEX 2T plasmid DNA (PharmaciaBiotech) using T-4 DNA ligase (New England Biolabs) as described by T. Maniatis, Molecular Cloning: A Laboratory Manual, 2nd ed. (1989). The ligation reaction was transformed into chemically competent E. coli DH10B cells purchased from Life-Technologies following the manufacturer's instructions. Plasmid DNA was isolated from the resulting bacterial colonies using a Promega Wizard™. miniprep kit. Plasmids containing the appropriate Bam HI fragment were sequenced in a DNA Thermal Cycler (Perkin Elmer) with Prism™. (Applied Biosystems Inc.). cDNA clones were identified that coded for both human p38a isoforms (Lee et al. Nature 372, 739). One of the clones that contained the cDNA for p38a-2 (CSB-2) inserted in the cloning site of PGEX 2T, 3′ of the GST coding region was designated pMON 35802. The sequence obtained for this clone is an exact match of the cDNA clone reported by Lee et al. This expression plasmid allows for the production of a GST-p38a fusion protein.

Expression of Human P38 Kinase-alpha

GST/p38a fusion protein w as expressed from the plasmid PMON 35802 in E. coli, stain DH10B (Life Technologies, Gibco-BRL). Overnight cultures were grown in Luria Broth (LB) containing 100 mg/ml ampicillin. The next day, 500 ml of fresh LB was inoculated with 10 ml of overnight culture, and grown in a 2 liter flask at 37° C. with constant shaking until the culture reached an absorbance of 0.8 at 600 nm. Expression of the fusion protein was induced by addition of isopropyl b-D-thiogalactosidase (IPTG) to a final concentration of 0.05 mM. The cultures were shaken for three hours at room temperature, and the cells were harvested by centrifugation. The cell pellets were stored frozen until protein purification.

Purification of P38 Kinase-alpha

All chemicals were from Sigma Chemical Co. unless noted. Twenty grams of E. coli cell pellet collected from five 1 L shake flask fermentations was resuspended in a volume of PBS (140 mM NaCl, 2.7 mM KCl, 10 mM Na.sub.2 HPO.sub.4, 1.8 mM KH.sub.2 PO.sub.4, pH 7.3) up to 200 ml. The cell suspension was adjusted to 5 mM DTT with 2 M DTT and then split equally into five 50 ml Falcon conical tubes. The cells were sonnicated (Ultrasonics model W375) with a 1 cm probe for 3.times.1 minutes (pulsed) on ice. Lysed cell material was removed by centrifugation (12,000×g, 15 minutes) and the clarified supernatant applied to glutathione-sepharose resin (Pharmacia).

Glutathione-Sepharose Affinity Chromatography

Twelve ml of a 50% glutathione sepharose-PBS suspension was added to 200 ml clarified supernatant and incubated batchwise for 30 minutes at room temperature. The resin was collected by centrifugation (600.times.g, 5 min) and washed with 2.times.150 ml PBS/1% Triton X-100, followed by 4.times.40 ml PBS. To cleave the p38 kinase from the GST-p38 fusion protein, the glutathione-sepharose resin was resuspended in 6 ml PBS containing 250 units thrombin protease (Pharmacia, specific activity >7500 units/mg) and mixed gently for 4 hours at room temperature. The glutathione-sepharose resin was removed by centrifugation (600.times.g, 5 min) and washed 2.times.6 ml with PBS. The PBS wash fractions and digest supernatant containing p38 kinase protein were pooled and adjusted to 0.3 mM PMSF.

Mono Q Anion Exchange Chromatography

The thrombin-cleaved p38 kinase was further purified by FPLC-anion exchange chromatography. Thrombin-cleaved sample was diluted 2-fold with Buffer A (25 mM HEPES, pH 7.5, 25 mM beta-glycerophosphate, 2 mM DTT, 5% glycerol) and injected onto a Mono Q HR 10/10 (Pharmacia) anion exchange column equilibrated with Buffer A. The column was eluted with a 160 ml 0.1 M-0.6 M NaCl/Buffer A gradient (2 ml/minute flowrate). The p38 kinase peak eluting at 200 mM NaCl was collected and concentrated to 3-4 ml with a Filtron 10 concentrator (Filtron Corp.).

Sephacryl S100 Gel Filtration Chromatography

The concentrated Mono Q-p38 kinase purified sample was purified by gel filtration chromatography (Pharmacia HiPrep 26/60 Sephacryl S100 column equilibrated with Buffer B (50 mM HEPES, pH 7.5, 50 mM NaCl, 2 mM DTT, 5% glycerol)). Protein was eluted from the column with Buffer B at a 0.5 ml/minute flowrate and protein was detected by absorbance at 280 nm. Fractions containing p38 kinase (detected by SDS-polyacrylamide gel electrophoresis) were pooled and frozen at −80° C. Typical purified protein yields from 5 L E. coli shake flasks fermentations were 35 mg p38 kinase.

In Vitro Assay

The ability of compounds to inhibit human p38 kinase alpha was evaluated using two in vitro assay methods. In the first method, activated human p38 kinase alpha phosphorylates a biotinylated substrate, PHAS-I (phosphorylated heat and acid stable protein-insulin inducible), in the presence of gamma ³²P-ATP (³²P-ATP). PHAS-I was biotinylated prior to the assay and provides a means of capturing the substrate, which is phosphorylated during the assay. p38 Kinase was activated by MKK6. Compounds were tested in 10 fold serial dilutions over the range of 100 μM to 0.001 μM using 1% DMSO. Each concentration of inhibitor was tested in triplicate.

All reactions were carried out in 96 well polypropylene plates. Each reaction well contained 25 mM HEPES pH 7.5, 10 mM magnesium acetate and 50.mu.M unlabeled ATP. Activation of p38 was required to achieve sufficient signal in the assay. Biotinylated PHAS-I was used at 1-2 μg per 50 μl reaction volume, with a final concentration of 1.5 μM. Activated human p38 kinase alpha was used at 1 μg per 50 μl reaction volume representing a final concentration of 0.3 μM. Gamma .sup.32 P-ATP was used to follow the phosphorylation of PHAS-I. .sup.32 P-ATP has a specific activity of 3000 Ci/mmol and was used at 1.2 μCi per 50 μl reaction volume. The reaction proceeded either for one hour or overnight at 30° C.

Following incubation, 20 μl of reaction mixture was transferred to a high capacity streptavidin coated filter plate (SAM-streptavidin-matrix, Promega) prewetted with phosphate buffered saline. The transferred reaction mix was allowed to contact the streptavidin membrane of the Promega plate for 1-2 minutes. Following capture of biotinylated PHAS-I with ³²P incorporated, each well was washed to remove unincorporated ³²P-ATP three times with 2M NaCl, three washes of 2M NaCl with 1% phosphoric, three washes of distilled water and finally a single wash of 95% ethanol. Filter plates were air-dried and 20 μl of scintillant was added. The plates were sealed and counted.

A second assay format was also employed that is based on p38 kinase alpha induced phosphorylation of EGFRP (epidermal growth factor receptor peptide, a 21 mer) in the presence of ³³P-ATP. Compounds were tested in 10 fold serial dilutions over the range of 100 μM to 0.001 μM in 1% DMSO. Each concentration of inhibitor was tested in triplicate. Compounds were evaluated in 50 μl reaction volumes in the presence of 25 mM Hepes pH 7.5, 10 mM magnesium acetate, 4% glycerol, 0.4% bovine serum albumin, 0.4 mM DTT, 50 μM unlabeled ATP, 25 μg EGFRP (200 μM), and 0.05 μCi gamma ³³P-ATP. Reactions were initiated by addition of 0.09 μg of activated, purified human GST-p38 kinase alpha. Activation was carried out using GST-MKK6 (5:1,p38:MKK6) for one hour at 30° C. in the presence of 50 μM ATP. Following incubation for 60 minutes at room temperature, the reaction was stopped by addition of 150 μl of AG 1.times.8 resin in 900 mM sodium formate buffer, pH 3.0 (1 volume resin to 2 volumes buffer). The mixture was mixed three times with pipetting and the resin was allowed to settle. A total of 50 μl of clarified solution head volume was transferred from the reaction wells to Microlite-2 plates. 150 μl of Microscint 40 was then added to each well of the Microlite plate, and the plate was sealed, mixed, and counted.

TNF Cell Assays

Method of Isolation of Human Peripheral Blood Mononuclear Cells:

Human whole blood was collected in Vacutainer tubes containing EDTA as an anticoagulant. A blood sample (7 ml) was carefully layered over 5 ml PMN Cell Isolation Medium (Robbins Scientific) in a 15 ml round bottom centrifuge tube. The sample was centrifuged at 450-500.times.g for 30-35 minutes in a swing out rotor at room temperature. After centrifugation, the top band of cells were removed and washed 3 times with PBS w/o calcium or magnesium. The cells were centrifuged at 400.times.g for 10 minutes at room temperature. The cells were resuspended in Macrophage Serum Free Medium (Gibco BRL) at a concentration of 2 million cells/mi.

LPS Stimulation of Human PBMs

PBM cells (0.1 ml, 2 million/ml) were co-incubated with 0.1 ml compound (10-0.41 μM, final concentration) for 1 hour in flat bottom 96 well microtiter plates. Compounds were dissolved in DMSO initially and diluted in TCM for a final concentration of 0.1% DMSO. LPS (Calbiochem, 20 ng/ml, final concentration) was then added at a volume of 0.010 ml. Cultures were incubated overnight at 37° C. Supernatants were then removed and tested by ELISA for TNF-a and IL1-b. Viability was analyzed using MTS. After 0.1 ml supernatant was collected, 0.020 ml MTS was added to remaining 0.1 ml cells. The cells were incubated at 37° C. for 2-4 hours, then the O.D. was measured at 490-650 nM.

Maintenance and Differentiation of the U937 Human Histiocytic Lymphoma Cell Line

U937 cells (ATCC) were propagated in RPMI 1640 containing 10% fetal bovine serum, 100 IU/ml penicillin, 100 μg/ml streptomycin, and 2 mM glutamine (Gibco). Fifty million cells in 100 ml media were induced to terminal monocytic differentiation by 24 hour incubation with 20 ng/ml phorbol 12-myristate 13-acetate (Sigma). The cells were washed by centrifugation (200.times.g for 5 min) and resuspended in 100 ml fresh medium. After 24-48 hours, the cells were harvested, centrifuged, and resuspended in culture medium at 2 million cells/ml.

LPS Stimulation of TNF Production by U937 Cells

U937 cells (0.1 ml, 2 million/ml) were incubated with 0.1 ml compound (0.004-50 μM, final concentration) for 1 hour in 96 well microtiter plates. Compounds were prepared as 10 mM stock solutions in DMSO and diluted in culture medium to yield a final DMSO concentration of 0.1% in the cell assay. LPS (E coli, 100 ng/ml final concentration) was then added at a volume of 0.02 ml. After 4 hour incubation at 37° C., the amount of TNF-.alpha. released in the culture medium was quantitated by ELISA. Inhibitory potency is expressed as IC50 (μM).

Rat Assay

The efficacy of the novel compounds in blocking the production of TNF also was evaluated using a model based on rats challenged with LPS. Male Harlen Lewis rats [Sprague Dawley Co.] were used in this model. Each rat weighed approximately 300 g and was fasted overnight prior to testing. Compound administration was typically by oral gavage (although intraperitoneal, subcutaneous and intravenous administration were also used in a few instances) 1 to 24 hours prior to the LPS challenge. Rats were administered 30 μg/kg LPS [salmonella typhosa, Sigma Co.] intravenously via the tail vein. Blood was collected via heart puncture 1 hour after the LPS challenge. Serum samples were stored at −20° C. until quantitative analysis of TNF-.alpha. by Enzyme Linked-Immuno-Sorbent Assay (“ELISA”) [Biosource]. Additional details of the assay are set forth in Perretti, M., et al., Br. J. Pharmacol. (1993), 110, 868-874, which is incorporated by reference in this application.

Mouse Assay

Mouse Model of LPS-Induced TNF Alpha Production

TNF alpha was induced in 10-12 week old BALB/c female mice by tail vein injection with 100 ng lipopolysaccharide (from S. Typhosa) in 0.2 ml saline. One hour later mice were bled from the retroorbital sinus and TNF concentrations in serum from clotted blood were quantified by ELISA. Typically, peak levels of serum TNF ranged from 2-6 ng/ml one hour after LPS injection.

The compounds tested were administered to fasted mice by oral gavage as a suspension in 0.2 ml of 0.5% methylcellulose and 0.025% Tween 20 in water at 1 hour or 6 hours prior to LPS injection. The 1 hour protocol allowed evaluation of compound potency at Cmax plasma levels whereas the 6 hour protocol allowed estimation of compound duration of action. Efficacy was determined at each time point as percent inhibition of serum TNF levels relative to LPS injected mice that received vehicle only.

Induction and Assessment of Collagen-Induced Arthritis in Mice

Arthritis was induced in mice according to the procedure set forth in J. M. Stuart, Collagen Autoimmune Arthritis, Annual Rev. Immunol. 2:199 (1984), which is incorporated herein by reference. Specifically, arthritis was induced in 8-12 week old DBA/1 male mice by injection of 50 μg of chick type II collagen (CII) (provided by Dr. Marie Griffiths, Univ. of Utah, Salt Lake City, Utah) in complete Freund's adjuvant (Sigma) on day 0 at the base of the tail. Injection volume was 100 μl. Animals were boosted on day 21 with 50 μg of CII in incomplete Freund's adjuvant (100 μl volume). Animals were evaluated several times each week for signs of arthritis. Any animal with paw redness or swelling was counted as arthritic. Scoring of arthritic paws was conducted in accordance with the procedure set forth in Wooley et al., Genetic Control of Type II Collagen Induced Arthritis in Mice: Factors Influencing Disease Suspectibility and Evidence for Multiple MHC Associated Gene Control., Trans. Proc., 15:180 (1983). Scoring of severity was carried out using a score of 1-3 for each paw (maximal score of 12/mouse). Animals displaying any redness or swelling of digits or the paw were scored as 1. Gross swelling of the whole paw or deformity was scored as 2. Ankylosis of joints was scored as 3. Animals were evaluated for 8 weeks. 8-10 animals per group were used.

TABLE 1
N-Benzyl Pyridazinones

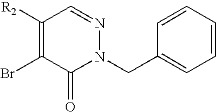

| R₂ group | Compound No. |
|---|---|
| 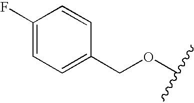 | 1 |
| 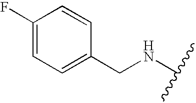 | 2 |
| 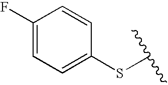 | 3 |
| 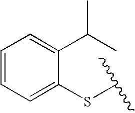 | 4 |
| 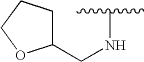 | 5 |
| 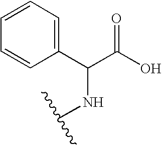 | 6 |
| 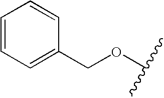 | 7 |
| 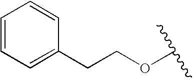 | 8 |

Compound 2 in Table 1 exhibits an IC₅₀ of 60-80 μM and compounds 1, 3-8 exhibit an IC₅₀ of >100 μM (p38 alpha kinase assay).

TABLE 2
N-Phenyl Pyridazinones

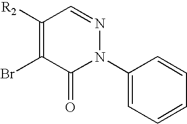

| R₂ group | Compound No. |
|---|---|
| 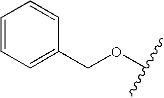 | 9 |

Compound 9 in Table 2 exhibits an IC₅₀ of 20-40 μM (p38 alpha kinase assay).

TABLE 3
N-2,6-Dichlorophenyl Pyridazinones

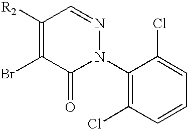

| R₂ group | Compound No. |
|---|---|
| 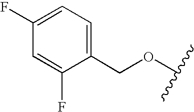 | 10 |
| 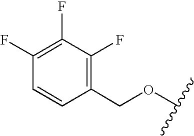 | 11 |
| 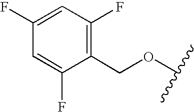 | 12 |
| 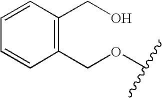 | 13 |
| 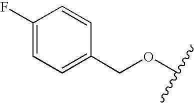 | 14 |
| 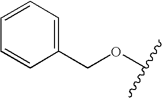 | 15 |
| 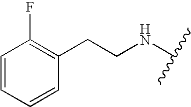 | 16 |
| 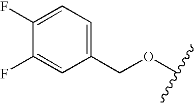 | 17 |
| 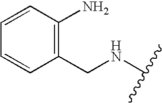 | 18 |
| 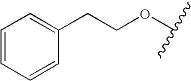 | 19 |
| 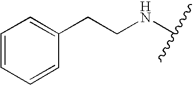 | 20 |
| 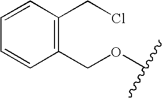 | 21 |
| 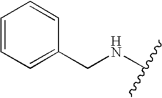 | 22 |
| 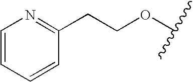 | 23 |
| 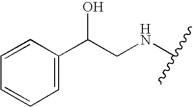 | 24 |
| 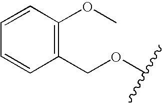 | 25 |
| 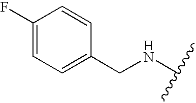 | 26 |
| 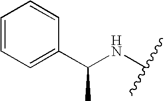 | 27 |
| 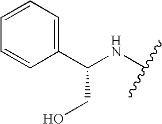 | 28 |
| 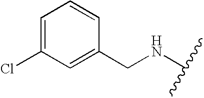 | 29 |
| 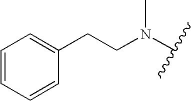 | 30 |
| 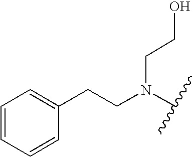 | 31 |
| 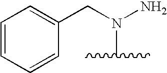 | 32 |
| 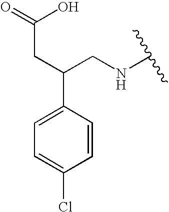 | 33 |
| 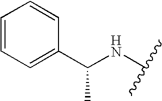 | 34 |
| 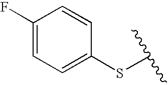 | 35 |
| 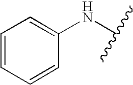 | 36 |
| 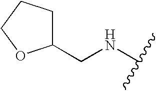 | 37 |
| 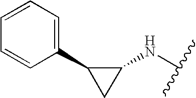 | 38 |
| 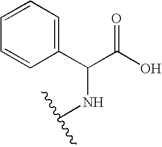 | 39 |
| 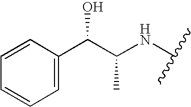 | 40 |
| 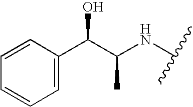 | 41 |
| 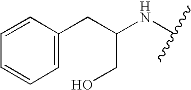 | 42 |
| 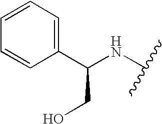 | 43 |
| 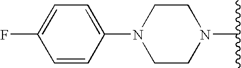 | 44 |
| 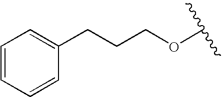 | 45 |
| 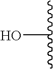 | 46 |
| 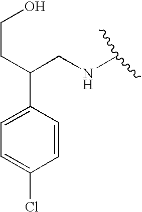 | 47 |

Compounds 10-28 in Table 3 exhibits an IC₅₀ of 0.1-20 μM, compounds 29-30 exhibit an IC₅₀ of 20-40 μM, compound 31 exhibits an IC₅₀ of 40-60 μM, compound 32 exhibits an IC₅₀ of 60-80 μM, compounds 33-34 exhibits an IC₅₀ of 80-100 μM, and compounds 35-47 exhibit an IC₅₀ of >100 μM, (p38 alpha kinase assay)

TABLE 4
N-2,6-Dichlorophenyl Pyridazinones

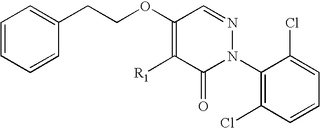

| R₁ group | Compound No. |
|---|---|
| H— | 48 |
| Me— | 49 |
| 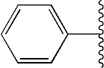 | 50 |
| 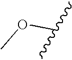 | 51 |
| 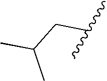 | 52 |
| 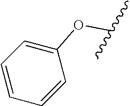 | 53 |

Compound 49 in Table 4 exhibits an IC₅₀ of 0.1-20 μM, compound 48 exhibits an IC₅₀ of 40-60 μM, compound 51 exhibits an IC₅₀ of 60-80 μM, and compounds 50, 52-3 exhibit an IC₅₀ of >100 μM, (p38 alpha kinase assay).

TABLE 5
5-Phenethyl ether N-2 Pyridazinones

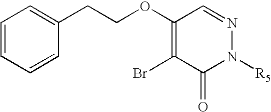

| R₅ group | Compound No. |
|---|---|
| —H | 54 |
| 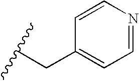 | 55 |
| 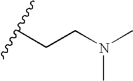 | 56 |
| 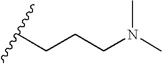 | 57 |
| 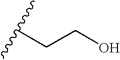 | 58 |

Compounds 54-58 in Table 5 exhibit an IC₅₀ of >100 μM, (p38 alpha kinase assay).

TABLE 6
5-(2,4-Difluorobenzyloxy) N-2 Pyridazinones

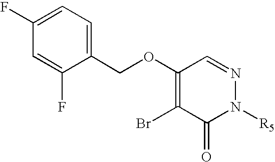

| R₅ group | Compound No. |
|---|---|
| H | 59 |
| 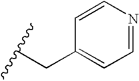 | 60 |
| 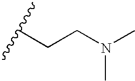 | 61 |
| 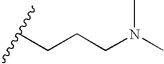 | 62 |
| 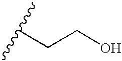 | 63 |
| 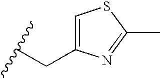 | 64 |

Compounds 60 and 64 in Table 6 exhibit an IC₅₀ of 0.1-20 μM, compound 63 exhibits an IC₅₀ of 20-40 μM, and compounds 59, 61-62 exhibit an IC₅₀ of >100 μM, (p38 alpha kinase assay).

TABLE 7

| | Compound |
|---|---|
| Structure | No. |
| 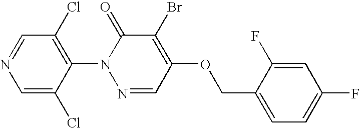 | 65 |
| 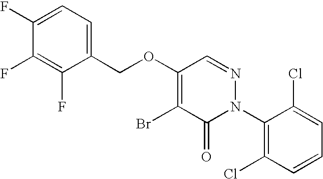 | 66 |
| 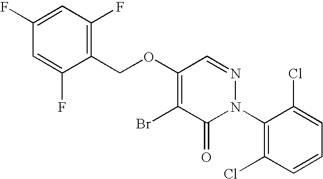 | 67 |
| 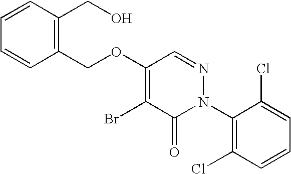 | 68 |
| 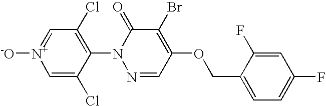 | 69 |
| 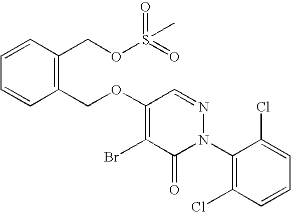 | 70 |
| 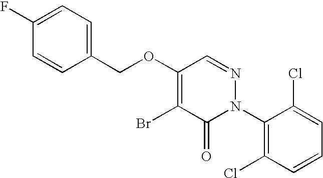 | 71 |
| 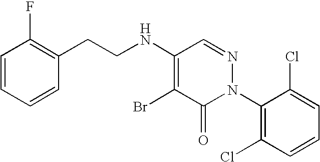 | 72 |
| 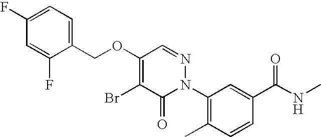 | 73 |

Compounds 65-72 in Table 7 exhibit an IC₅₀ of 0.1-20 μM (p38 alpha kinase assay).

Preparation and Administration of Compounds

The compounds tested on mice having collagen-induced arthritis were prepared as a suspension in 0.5% methylcellulose (Sigma, St. Louis, Mo.), 0.025% Tween 20 (Sigma). The compound suspensions were administered by oral gavage in a volume of 0.1 ml b.i.d. Administration began on day 20 post collagen injection and continued daily until final evaluation on day 56. Scoring of arthritic paws was conducted as set forth above.

The compounds of the invention interact with the p38 alpha and p38 beta MAP kinases. Preferably, Compounds of the invention have activities in assays for these enzymes less than approximately 500 micromolar and more preferably 100 micromolar.

The compound names in this application were generated using ACD Name Pro program, version 5.09. Make sure all compounds are named.

For oral administration, the pharmaceutical composition may be in the form of, for example, a tablet, hard or soft capsule, lozenges, dispensable powders, suspension, or liquid. The pharmaceutical composition is preferably made in the form of a dosage unit containing a particular amount of the active ingredient. Examples of such dosage units are tablets or capsules.

The active ingredient may also be administered by injection (IV, IM, subcutaneous or jet) as a composition wherein, for example, saline, dextrose, or water may be used as a suitable carrier. The pH of the composition may be adjusted, if necessary, with suitable acid, base, or buffer. Suitable bulking, dispersing, wetting or suspending agents, including mannitol and PEG 400, may also be included in the composition. A suitable parenteral composition can also include a compound formulated as a sterile solid substance, including lyophilized powder, in injection vials. Aqueous solution can be added to dissolve the compound prior to injection.

The amount of therapeutically active compounds that are administered and the dosage regimen for treating a disease condition with the compounds and/or compositions of this invention depends on a variety of factors, including the age, weight, sex and medical condition of the subject, the severity of the inflammation or inflammation related disorder, the route and frequency of administration, and the particular compound employed, and thus may vary widely. The pharmaceutical compositions may contain active ingredients in the range of about 0.1 to 1000 mg, preferably in the range of about 7.0 to 350 mg. A daily dose of about 0.01 to 100 mg/kg body weight, preferably between about 0.1 and about 50 mg/kg body weight and most preferably between about 0.5 to 30 mg/kg body weight, may be appropriate. The daily dose can be administered in one to four doses per day. In the case of skin conditions, it may be preferable to apply a topical preparation of compounds of this invention to the affected area two to four times a day.

For disorders of the eye or other external tissues, e.g., mouth and skin, the formulations are preferably applied as a topical gel, spray, ointment, or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound, which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base, which forms the oily, dispersed phase of the cream formulations. Emulsifiers and emulsion stabilizers suitable for use in the formulation of the invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono-or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The antiinflammatory active ingredients are preferably present in such formulations in a concentration of 0.5 to 20%, advantageously 0.5 to 10% and particularly about 1.5% wlw. For therapeutic purposes, the active compounds of this combination invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The invention has been described with reference to various specific and preferred embodiments and techniques. However, it should be understood that many variations and modifications may be made while remaining within the spirit and scope of the invention.

1. A compound of Formula I: 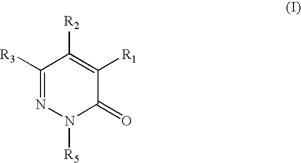
or a pharmaceutically acceptable salt thereof, wherein
R₁ is H, halogen, NO₂, alkyl, carboxaldehyde, hydroxyalkyl, dihydroxyalkyl, arylalkoxy, arylalkyl, alkenyl, alkynyl, arylalkynyl, —CN, aryl, alkanoyl, alkoxy, alkoxyalkyl, haloalkyl, haloalkoxy, carboxyl, aryloxy(Cₗ-C₆)alkyl, or arylalkanoyl,
wherein the aryl portion of arylalkoxy, arylalkyl, and arylalkanoyl is unsubstituted or substituted with 1, 2, 3, 4, or 5 groups that are independently halogen, C₁-C₄ alkyl, C₁-C₄ alkoxy, nitro, CN, haloalkyl, haloalkoxy or CO₂R;
wherein the alkyl portion of the alkyl, hydroxyalkyl, dihydroxyalkyl, arylalkoxy, aryloxy(C₁-C₆) alkyl, arylalkyl, alkanoyl, alkoxy, alkoxyalkyl and arylalkanoyl groups is unsubstituted or substituted with 1, 2, or 3 groups that are independently halogen, C₁-C₄ alkoxy, C₁-C₄ alkoxycarbonyl, or C₃-C₇ cycloalkyl;
R₂ is H, OH, halogen, —OSO₂—(C₁-C₆) alkyl, —OSO₂-aryl, arylalkoxy, heteroarylalkoxy, aryloxy, arylthio, arylalkylthio, arylamino (C₁-C₆) alkyl, arylalkylamino, arylthioalkoxy, arylalkynyl, alkoxy, aryloxy(C₁-C₆)alkyl, alkyl, alkynyl, —OC(O)NH(CH₂)ₙaryl, —OC(O)N(alkyl)(CH₂)ₙaryl, alkoxyalkoxy, dialkylamino, alkyl, alkoxy, aryl, arylalkyl, heteroaryl, heteroarylalkyl, arylalkenyl, heterocycloalkyl, heterocycloalkylalkyl, alkoxyalkoxy, NR₈R₉, dialkylamino, or CO₂R, wherein
n is 0, 1, 2, 3, 4, 5 or 6;
each of which groups is unsubstituted or substituted with 1, 2, 3, 4, or 5 groups that are independently halogen, —(C₁-C₆)alkyl-N(R)—CO₂R₃₀, haloalkyl, heteroaryl, heteroarylalkyl, —(C₁-C₆alkyl)-C(O)—NR₆R₇, —NR₆R₇, R₆R₇N—(C₁-C₆ alkyl)-, —C(O)NR₆R₇, —(C₁-C₄ alkyl)-NRC(O)NR₁₆R₁₇, —(C₁-C₄) alkyl-OSO₂—(C₁-C₆)alkyl, haloalkoxy, alkyl, CN, hydroxyalkyl, dihydroxyalkyl, alkoxy, alkoxycarbonyl, phenyl, —SO₂-phenyl wherein the phenyl and —SO₂-phenyl groups are optionally substituted with 1, 2, or 3 groups that are independently halogen or NO₂, or —OC(O)NR₆R₇, wherein R₁₆ and R₁₇ are independently H or C₁-C₆ alkyl; or R₁₆, R₁₇ and the nitrogen to which they are attached form a morpholinyl ring;
R₆ and R₇ are independently at each occurrence H, alkyl, hydroxyalkyl, dihydroxyalkyl, alkoxy, alkanoyl, arylalkyl, arylalkoxy, alkoxycarbonyl, —SO₂-alkyl, OH, alkoxy, alkoxyalkyl, arylalkoxycarbonyl, —(C₁-C₄) alkyl-CO₂-alkyl, heteroarylalkyl, or arylalkanoyl, wherein each is unsubstituted or substituted with 1, 2, or 3 groups that are independently, halogen, OH, SH, heterocycloalkyl, heterocycloalkylalkyl, C₃-C₇ cycloalkyl, alkoxy, NH₂, NH(alkyl), N(alkyl)(alkyl), —O-alkanoyl, alkyl, haloalkyl, carboxaldehyde, or haloalkoxy; or
RCO₆, R₇, and the nitrogen to which they are attached form a morpholinyl, pyrrolidinyl, thiomorpholinyl, thiomorpholinyl S-oxide, thiomorpholinyl S,S-dioxide, piperidinyl, pyrrolidinyl, or piperazinyl ring which is optionally substituted with 1 or 2 groups that are independently C₁-C₄ alkyl, alkoxycarbonyl, C₁-C₄ alkoxy, hydroxyl, hydroxyalkyl, dihydroxyalkyl, or halogen;
R at each occurrence is independently hydrogen or C₁-C₆ alkyl optionally substituted with 1 or 2 groups that are independently OH, SH, halogen, amino, monoalkylamino, dialkylamino or C₃-C₆ cycloalkyl;
R₃₀ is C₁-C₆ alkyl optionally substituted with 1 or 2 groups that are independently OH, SH, halogen, amino, monoalkylamino, dialkylamino or C₃-C₆ cycloalkyl;
each R₈ is independently hydrogen, alkyl, alkanoyl, arylalkyl and arylalkanoyl, wherein each of the above is optionally substituted with 1, 2, 3, 4, or 5 groups that are independently alkyl, alkoxy, alkoxycarbonyl, halogen, or haloalkyl;
each R₉ is hydrogen, alkyl, alkanoyl, arylalkyl, cycloalkyl, arylcycloalkyl, cycloalkylalkyl, heterocycloalkylalkyl, arylheterocycloalkyl, alkenyl, heteroaryl, amino, aminoalkyl, monoalkylaminoalkyl, dialkylaminoalkyl, arylalkanoyl, —SO₂-phenyl, and aryl wherein each of the above is optionally substituted with 1, 2, 3, 4, or 5 groups that are independently alkyl, alkoxy, hydroxy, hydroxyalkyl, amino, —(CH₂)₀₋₄—COOR, alkoxycarbonyl, halogen, or haloalkyl;
R₃ is H, halogen, alkoxycarbonyl, arylalkoxycarbonyl, aryloxycarbonyl, arylalkyl, —OC(O)NH(CH₂)COₙaryl, arylalkoxy, —OC(O)N(alkyl) (CH₂)ₙaryl, aryloxy, arylthio, thioalkoxy, arylthioalkoxy, alkenyl, —COOR, hydroxyalkyl, arylalkylcarbonyl, arylalkoxyalkyl, —NR₆R₇, —C(O)NR₆R₇, NR₆R₇—(C₁-C₆)alkyl, or alkyl, wherein
the aryl portion of arylalkoxycarbonyl, aryloxycarbonyl, arylalkyl, —OC(O)NH(CH₂)ₙaryl, arylalkoxy, —OC(O)N(alkyl)(CH₂)ₙaryl, arylalkoxyalkyl, and arylthioalkoxy, is unsubstituted or substituted with 1, 2, 31, 4, or 5 groups that are independently, halogen, alkoxy, alkyl, haloalkyl, or haloalkoxy, wherein n is 0, 1, 2, 3, 4, 5, or 6; and
R₅ is H, aryl, heteroaryl, arylalkyl, arylthioalkyl, alkyl optionally substituted with 1, 2, or 3 groups that are independently arylalkoxycarbonyl, —NR₈R₉, halogen, —C(O)NR₈R₉, alkoxycarbonyl, C₃-C₇ cycloalkyl, or alkanoyl, alkoxy, alkoxyalkyl optionally substituted with one trimethylsilyl group, amino, alkoxycarbonyl, hydroxyalkyl, dihydroxyalkyl, alkynyl, —SO₂-alkyl, alkoxy optionally substituted with one trimethylsilyl group, heterocycloalkylalkyl, cycloalkyl, cycloalkylalkyl, —alkyl-S-aryl, -alkyl-SO₂-aryl, heteroarylalkyl, heterocycloalkyl, heteroaryl, or alkenyl optionally substituted with alkoxycarbonyl, wherein
each of the above is unsubstituted or substituted with 1, 2, 3, 4, or 5 groups that are independently alkyl, halogen, alkoxy, hydroxyalkyl, dihydroxyalkyl, arylalkoxy, thioalkoxy, alkoxycarbonyl, arylalkoxycarbonyl, CO₂R, CN, OH, hydroxyalkyl, dihydroxyalkyl, amidinooxime, —NR₆R₇, —NR₈R₉, R₆R₇N—(C₁-C₆ alkyl)-, carboxaldehyde, SO₂alkyl, —SO₂H, —SO₂NR₆R₇, alkanoyl wherein the alkyl portion is optionally substituted with OH, halogen or alkoxy, —C(O)NR₆R₇, —(C₁-C4 alkyl) —C(O)NR₆R₇, amidino, haloalkyl, —(C₁-C₄ alkyl) —NR₁₅C(O)NR₁₆R₁₇, —(C₁-C₄ alkyl) —NR₁₅C(O)R₁₈, —O-CH₂—O, —O—CH₂CH₂—O—, or haloalkoxy; wherein
R₁₅ is H or C₁-C₆ alkyl;
R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl,
provided that no more than two of R₁, R₂, and R₅ are simultaneously hydrogen.

2. A compound according to claim 1, of the formula: 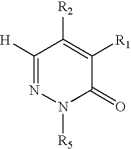
or a pharmaceutically acceptable salt thereof, wherein
R₁ is H, halogen, alkyl, carboxaldehyde, hydroxyalkyl, dihydroxyalkyl, arylalkoxy, arylalkyl, alkenyl, alkynyl, arylalkynyl, CN, alkanoyl, alkoxy, alkoxyalkyl, haloalkyl, carboxyl, or arylalkanoyl,
wherein the aryl portion of arylalkoxy, arylalkyl, and arylalkanoyl is unsubstituted or substituted with 1, 2, 3, 4, or 5 groups that are independently halogen, C₁-C₄ alkyl, C₁-C₄ alkoxy, nitro, CN, haloalkyl, haloalkoxy or CO₂R;
wherein the alkyl portion of the alkyl, hydroxyalkyl, dihydroxyalkyl, arylalkoxy, arylalkyl, alkanoyl, alkoxy, alkoxyalkyl and arylalkanoyl groups is unsubstituted or substituted with 1, 2, or 3 groups that are independently halogen, C₁-C₄ alkoxy, C₁-C₄ alkoxycarbonyl, or cyclopropyl;
R₂ is H, OH, halogen, —OSO₂—(C₁-C₆)alkyl, —OSO₂-aryl, arylthio, arylalkylthio, arylamino (C₁-C₆) alkyl, arylalkylamino, arylalkoxy, aryloxy, arylthioalkoxy, arylalkynyl, alkoxy, phenyloxy(C₁-C₆)alkyl, —OC(O)NH(CH₂)ₙaryl, —OC(O)N(alkyl)(CH₂)ₙaryl, alkyl, alkynyl, alkoxyalkoxy, dialkylamino, heteroaryl, heterocycloalkyl, aryloxyalkyl, or CO₂R, wherein
each of the above is unsubstituted or substituted with 1, 2, 3, 4, or 5 groups that are independently halogen, -NR₆R₇, haloalkyl, haloalkoxy, alkyl, heteroaryl, heteroarylalkyl, —(C₁-C₆alkyl)-C(O)—NR₆R₇, R₆R₇N—(C₁-C₆alkyl)-, —C(O)NR₆R₇, —(C₁-C₄ alkyl)-NRC(O)NR₁₆R₁₇, CN, hydroxyalkyl, dihydroxyalkyl, —OC(O)NR₆R₇, or —(C₁-C₆)alkyl-N(R)—CO₂R₃₀, wherein
R₁₆ and R₁₇ are independently H or C₁-C₆ alkyl; or
R₁₆, R₁₇ and the nitrogen to which they are attached form a morpholinyl ring;
R₆ and R₇ are independently at each occurrence H, alkyl, hydroxyalkyl, dihydroxyalkyl, alkoxy, alkoxyalkyl, alkanoyl, arylalkyl, arylalkoxy, arylalkoxycarbonyl, or arylalkanoyl, wherein each of the above is unsubstituted or substituted with 1, 2, or 3 groups that are independently, halogen, alkoxy, alkyl, OH, SH, carboxaldehyde, haloalkyl, or haloalkoxy; or
R₆, R₇, and the nitrogen to which they are attached form a morpholinyl, thiomorpholinyl, thiomorpholinyl S-oxide, thiomorpholinyl S,S-dioxide, piperidinyl, pyrrolidinyl, or piperazinyl ring which is optionally substituted with 1 or 2 groups that are independently C₁-C₄ alkyl, alkoxycarbonyl, hydroxyl, hydroxyalkyl, dihydroxyalkyl, or halogen;
n is 0, 1, 2, 3, 4, 5 or 6;
R at each occurrence is independently H or C₁-C₆ alkyl optionally substituted with 1 or 2 groups that are independently OH, SH, halogen, amino, monoalkylamino, dialkylamino or C₃-C₆ cycloalkyl;
R₃₀ is C₁-C₆ alkyl optionally substituted with 1 or 2 groups that are independently OH, SH, halogen, amino, monoalkylamino, dialkylamino or C₃-C₆ cycloalkyl; and
R₅ is H, arylalkyl, alkyl optionally substituted with 1, 2, or 3 groups that are independently arylalkoxycarbonyl, —NR₈R₉, halogen, —C(O)NR₈R₉, alkoxycarbonyl, or alkanoyl, alkoxyalkyl optionally substituted with one trimethylsilyl group, alkoxycarbonyl, amino, hydroxyalkyl, dihydroxyalkyl, alkenyl optionally substituted with alkoxycarbonyl, alkynyl, —SO₂-alkyl, aryl, alkoxy optionally substituted with one trimethylsilyl group, heterocycloalkylalkyl, heteroarylalkyl, heterocycloalkyl, or heteroaryl, wherein
each of the above is unsubstituted or substituted with 1, 2, 3, 4, or 5 groups that are independently alkyl, halogen, alkoxy, arylalkoxy, hydroxyalkyl, dihydroxyalkyl, thioalkoxy, —SO₂alkyl, alkoxycarbonyl, arylalkoxycarbonyl, CO₂R, CN, OH, amidinooxime, NR₈R₉, R₆R₇N—(C₁-C₆ alkyl)-, —C(O)NR₆R₇, amidino, hydroxyalkyl, dihydroxyalkyl, carboxaldehyde, —NR₆R₇, haloalkyl, —(C₁-C₄ alkyl)-C(O)NR₆R₇, —(C₁-C₄ alkyl)-CO₂R, —(C₁-C₄ alkyl)-C₁-C₆ alkoxycarbonyl, —(C₁-C₄ alkyl)-CN, —(C₁-C₄ alkyl)-NR₁₅C(O)R₁₈, —O—CH₂—O—, —O—CH₂CH₂—O—, phenyl or haloalkoxy;
R₈ is hydrogen, alkyl, alkanoyl, arylalkyl and arylalkanoyl;
R₉ is alkyl, alkanoyl, arylalkyl, heteroaryl, aminoalkyl, monoalkylaminoalkyl, dialkylaminoalkyl, and arylalkanoyl.

3. A compound according to claim 2 wherein
R₁ is H, halogen, alkyl optionally substituted with C₁-C₄ alkoxycarbonyl, carboxaldehyde, hydroxyalkyl, dihydroxyalkyl, phenyl (C₁-C₆) alkoxy, phenyl (C₁-C₆) alkyl, CN, alkanoyl, alkoxy, C₂-C₄ alkynyl, C₂-C₆ alkenyl optionally substituted with C₁-C₄ alkoxycarbonyl, alkoxyalkyl, haloalkyl, or phenyl (C₁-C₆)alkanoyl,
wherein the phenyl groups are unsubstituted or substituted with 1, 2, 3, 4, or 5 groups that are independently halogen, C₁-C₄ alkyl, C₁-C₄ alkoxy, nitro, CN, CF₃, OCF₃ or CO₂R;
wherein the alkyl groups are unsubstituted or substituted with 1, 2, or 3 groups that are independently halogen, methoxy, or ethoxy;
R₂ is OH, phenyl(C₁-C₆)alkoxy, phenyloxy, phenyloxy(C₁-C₆)alkyl, phenylthio, phenylalkylthio, phenylamino (C₁-C₆)alkyl, phenylalkylamino, phenyl (C₁-C₄) thioalkoxy, C₁-C₈ alkoxy, alkoxyalkoxy, —O—SO₂phenyl, alkynyl, phenyl (C₂-C₄) alkynyl, alkyl, —OC(O)NH(CH₂),phenyl, —OC(O)N(alkyl) (CH₂)ₙphenyl, dialkylamino, pyridyl, pyrimidyl, pyridazyl, pyrazolyl, imidazolyl, pyrrolyl, tetrahydroquinolinyl, tetrahydroisoquinolinyl, tetrazolyl, pyrazinyl, benzimidazolyl, triazinyl, tetrahydrofuryl, piperidinyl, hexahydropyrimidinyl, thiazolyl, thienyl, or CO₂R, wherein
n is 0, 1, 2, 3, 4, 5 or 6;
each of the above is unsubstituted or substituted with 1, 2, 3, 4, or 5 groups that are independently halogen, NR₆R₇, haloalkyl, haloalkoxy, hydroxyalkyl, dihydroxyalkyl, alkyl, phenyl, pyridyl, piperidinyl, piperazinyl, —(C₁-C₆alkyl)-C(O)—NR₆R₇, —(C₁-C₆)alkyl-N(R)—CO₂R₃₀, R₆R₇N—(C₁-C₆ alkyl)-, —C(O)NR₆R₇, —(C₁-C₄ alkyl)-NRC(O)NR₁₆R₁₇, or —OC(O)NR₆R₇, wherein
R₆ and R₇ are independently at each occurrence H, alkyl, (C₁-C₄) hydroxyalkyl, (C₁-C₄) dihydroxyalkyl, (C₁-C₄) alkoxy, (C₁-C₄) alkoxy (C₁-C₄) alkyl, (C₁-C₄) alkanoyl, phenyl (C₁-C₄) alkyl, phenyl (C₁-C₄) alkoxy, phenyl (C₁-C₄) alkoxycarbonyl, or phenyl (C₁-C₄) alkanoyl, wherein each of the above is unsubstituted or substituted with 1, 2, or 3 groups that are independently, halogen, OH, SH, C₃-C₆ cycloalkyl, (C₁-C₄) alkoxy, (C₁-C₄) alkyl, CF₃, carboxaldehyde, NH₂, NH(C₁-C₆)alkyl, N(C₁-C₆)alkyl (C₁-C₆)alkyl, OCF₃; or
R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, hydroxy, hydroxy C₁-C₄ alkyl, C₁-C₄ dihydroxyalkyl, C₁-C₄ alkoxycarbonyl, or halogen; and
R₅ is phenyl(C₁-C₆)alkyl, (C₁-C₆)alkyl optionally substituted with 1, 2, 3, 4, or 5 groups that are independently phenyl C₁-C₄ alkoxycarbonyl, —NR₈R₉, halogen, —C(O)NR₈R₉, alkoxycarbonyl, or alkanoyl, phenyl, alkoxy, C₂-C₆ alkynyl, C₂-C₆ alkenyl optionally substituted with alkoxycarbonyl, indolyl, quinolinyl, isoquinolinyl, isoindolyl, dihydroindolyl, dihydroisoindolyl, indolon-2-yl, indazolyl, benzimidazolyl, pyridyl, imidazolidine dione, pyridyl (C₁-C₆) alkyl, pyridazinyl (C₁-C₆) alkyl, pyrimidinyl (C₁-C₆) alkyl, pyrazinyl (C₁-C₆) alkyl, tetrahydrofuryl (C₁-C₆)alkyl, naphthyl (C₁-C₆)alkyl, morpholinyl (C₁-C₆) alkyl, tetrahydrofuryl (C₁-C₆) alkyl, thienyl (C₁-C₆) alkyl, piperazinyl (C₁-C₆) alkyl, indolyl (C₁-C₆) alkyl, quinolinyl(C₁-C₆) alkyl, isoquinolinyl(C₁-C₆) alkyl, isoindolyl(C₁-₆) alkyl, dihydroindolyl(C₁-C₆) alkyl, dihydroisoindolyl(C₁-C₆) alkyl, indoon-2-yl(C₁-C₆) alkyl, indolon-2-yl(C₁-C₆) alkyl, or morpholinyl C₁-C₆ alkyl, wherein
each of the above is unsubstituted or substituted with 1, 2, 3, 4, or 5 groups that are independently C₁-C₆ alkyl, halogen, C₁-C₆ alkoxy, phenyl C₁-C₆ alkoxy, C₁-C₆ thioalkoxy, C₁-C₆ alkoxycarbonyl, CO₂R, CN, —SO₂(C₁-C₆)alkyl, amidinooxime, NR₈R₉, —NR₆R₇, NR₆R₇ C₁-C₆ alkyl, —C(O)NR₆R₇, amidino, —(C₁-C₆alkyl)-C(O)—NR₆R₇, C₁-C₄ haloalkyl, hydroxy C₁-C₆ alkyl, C₁-C₆ dihydroxyalkyl, or C₁-C₄ haloalkoxy; wherein
R₈ is hydrogen, C₁-C₆ alkyl, C₁-C₆ alkanoyl, phenyl C₁-C₆ alkyl and phenyl C₁-C₆ alkanoyl; and
R₉ is aminoalkyl, mono C₁-C₆ alkylamino C₁-C₆ alkyl, di C₁-C₆ alkylamino C₁-C₆ alkyl, C₁-C₆ alkyl, C₁-C₆ alkanoyl, phenyl C₁-C₆ alkyl, indazolyl, and phenyl C₁-C₆ alkanoyl.

4. A compound according to claim 3, wherein
R₁ is H, halogen, C₁-C₄ alkyl optionally substituted with C₁-C₄ alkoxycarbonyl, C₂-C₄ alkenyl optionally substituted with C₁-C₄ alkoxycarbonyl, C₂-C₄ alkynyl, or carboxaldehyde;
R₂ is benzyloxy, OH, phenyloxy, phenyloxy(C₁-C₆)alkyl, phenyl (C₁-C₄) thioalkoxy, or pyridyl; wherein each of the above is optionally substituted with 1, 2, 3, 4, or 5 groups that are independently halogen, —(C₁-C₆)alkyl-N(R)—CO₂R₃₀, —(C₁-C₆alkyl) —C(O)—NR₆R₇, NR₆R₇, (C₁-C₄) haloalkyl, —C(O)NR₆R₇, —(C₁-C₄ alkyl)-NRC(O)NR₁₆R_{17,} (C₁-C₄) haloalkoxy, hydroxyalkyl, C₁-C₆ dihydroxyalkyl, (C₁-C₆) alkyl, pyridyl, or R₆R₇N—(C₁-C₆ alkyl)-.

5. A compound according to claim 4, wherein
R₅ is indolyl, pyridyl, pyridazinyl, pyrimidinyl, indazolyl, quinolinyl, isoquinolinyl, isoindolyl, dihydroindolyl, dihydroisoindolyl, indolon-2-yl, pyridazinyl, pyrimidinyl, or pyrazinyl, each of which is unsubstituted or substituted with 1, 2, 3, 4 or 5 groups that are independently C₁-C₄ alkyl, halogen, CF₃, OCF₃, —CO₂CH₃, C₁-C₄ hydroxyalkyl, dihydroxyalkyl, C₁-C₄ alkoxy, —CO₂(C₁-C₅ alkyl), benzyloxy, —NR₆R₇, —NR₈R₉, NR₆R₇—(C₁-C₄ alkyl), —C(O)NR₆R₇, or amidinooxime; wherein
R₆ and R₇ are independently at each occurrence H, C₁-C₄ alkyl, C₁-C₄ hydroxyalkyl, C₁-C₄ dihydroxyalkyl, C₁-C₄ alkoxy, C₁-C₄ alkoxy C₁-C₄ alkyl, C₁-C₄ alkanoyl, phenyl C₁-C₄ alkyl, phenyl C₁-C₄ alkoxy, or phenyl C₁-C₄ alkanoyl, wherein each is unsubstituted or substituted with 1, 2, or 3 groups that are independently, halogen, OH, SH, C₃-₆ cycloalkyl, C₁-C₄ alkoxy, C₁-C₄ alkyl, OH, CF₃, or OCF₃; or
R₆, R₇, and the nitrogen to which they are attached form a morpholinyl, thiomorpholinyl, pyrrolidinyl, or piperazinyl ring which is optionally substituted with 1 or 2 groups that are independently C₁-C₄ alkyl, hydroxy, hydroxy C₁-C₄ alkyl, C₁-C₄ dihydroxyalkyl, or halogen.

6. A compound according to claim 5, wherein
R₅ is indolyl, pyridyl, pyrimidinyl, indazolyl, dihydroindolyl, dihydroisoindolyl, indolon-2-yl, or pyrazinyl, each of which is unsubstituted or substituted with 1, 2, 3, or 4 groups that are independently C₁-C₄ alkyl, halogen, CF₃, OCF₃, —CO₂CH₃, C₁-C₄ hydroxyalkyl, C₁-C₄ dihydroxyalkyl, C₁-C₄ alkoxy, —CO₂(C₁-C₅ alkyl), benzyloxy, —C(O)NR₆R₇, —NR₈R₉, —NR₆R₇, NR₆R₇—(C₁-C₄ alkyl)-, and amidinooxime.

7. A compound according to claim 6, wherein
R₅ is indolyl, pyridyl, pyrimidinyl, dihydroindolyl, or pyrazinyl, each of which is unsubstituted or substituted with 1, 2, 3, or 4 groups that are independently C₁-C₄ alkyl, halogen, CF₃, OCF₃, —CO₂CH₃, C₁-C₄ hydroxyalkyl, C₁-C₄ dihydroxyalkyl, C₁-C₄ alkoxy, —CO₂(C₁-C₅ alkyl), benzyloxy, —C(O)NR₆R—, NR₈R₉, —NR₆R₇, NR₆R₇—(C₁-C₄ alkyl)-, or amidinooxime; wherein
R₆ and R₇ are independently at each occurrence H, C₁-C₄ alkyl, C₁-C₄ hydroxyalkyl, C₁-C₄ dihydroxyalkyl, C₁-C₄ alkoxy, C₁-C₄ alkanoyl, C₁-C₄ alkoxy C₁-C₄ alkyl, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃.

8. A compound according to claim 7, wherein
R₅ is indolyl, pyridyl, pyrimidinyl, dihydroindolyl, or pyrazinyl, each of which is unsubstituted or substituted with 1, 2, or 3 groups that are independently C₁-C₄ alkyl, halogen, CF₃, OCF₃, C₁-C₄ hydroxyalkyl, C₁-C₄ dihydroxyalkyl, C₁-C₄ alkoxy, —C(O)NR₆R₇, NR₈R₉, —NR₆R₇, or NR₆R₇—(C₁-C₄ alkyl)-; wherein
R₆ and R₇ are independently at each occurrence H, C₁-C₄ alkyl, C₁-C₄ hydroxyalkyl, C₁-C₄ dihydroxyalkyl, C₁-C₄ alkanoyl, or C₁-C₄ alkoxy, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃.

9. A compound according to claim 4, wherein
R₅ is phenyl(C₁-C₆)alkyl, or (C₁-C₆)alkyl, wherein
each of the above is unsubstituted or substituted with 1, 2, 3, 4, or 5 groups that are independently alkyl, halogen, alkoxy, benzyloxy, hydroxyalkyl, dihydroxyalkyl, thioalkoxy, —CO₂(C₁-C₅ alkyl), CO₂R, CN, amidinooxime, —NR₈R₉, —NR₆R₇, R₆R₇N—(C₁-C₆ alkyl)-, —C(O)NR₆R₇, amidino, CF₃, or OCF₃;
R₈ is hydrogen, C₁-₆ alkyl, C₁-₆ alkanoyl, phenyl C₁-₆ alkyl and phenyl C₁-₆ alkanoyl; and
R₉ is aminoalkyl, mono C₁-₆ alkylamino C₁-₆ alkyl, di C₁-C₆ alkylamino C₁-C₆ alkyl, C₁-C₆ alkyl, C₁-C₆ alkanoyl, phenyl C₁-C₄ alkyl, indazolyl, and phenyl C₁-C₄ alkanoyl.

10. A compound according to claim 4, wherein
R₅ is phenyl(C₁-C₆)alkyl, which is unsubstituted or substituted with 1, 2, 3, 4, or 5 groups that are independently alkyl, halogen, alkoxy, benzyloxy, thioalkoxy, —CO₂(C₁-C₅ alkyl), CO₂R, CN, amidinooxime, —NR₈R₉, —NR₆R₇, R₆R₇N—(C₁-C₆ alkyl)-, —C(O)NR₆R₇, amidino, CF₃, or OCF₃; wherein
R₆ and R₇ are independently at each occurrence H, C₁-C₄ alkyl, C₁-C₄ hydroxyalkyl, C₁-C₄ dihydroxyalkyl, C₁-C₄ alkoxy, C₃-C₄ alkoxy C₁-C₄ alkyl, C₁-C₄ alkanoyl, phenyl C₁-C₄ alkyl, phenyl C₁-C₄ alkoxy, or phenyl C₁-C₄ alkanoyl, wherein each is unsubstituted or substituted with 1, 2, or 3 groups that are independently, halogen, OH, SH, C₃-C₆ cycloalkyl, C₁-C₄ alkoxy, C₁-C₄ alkyl, CF₃, or OCF₃; or
R₆, R₇, and the nitrogen to which they are attached form a morpholinyl, thiomorpholinyl, or piperazinyl ring which is optionally substituted with 1 or 2 groups that are independently C₁-C₄ alkyl, hydroxy, hydroxy C₁-C₄ alkyl, C₁-C₄ dihydroxyalkyl, or halogen;
R₈ is hydrogen, C₁-C₆ alkyl, C₁-C₆ alkanoyl, phenyl C₁-C₆ alkyl and phenyl C₁-C₆ alkanoyl; and
R₉ is aminoalkyl, mono C₁-C₆ alkylamino C₁-C₆ alkyl, di C₁-C₆ alkylamino C₁-C₆ alkyl, C₁-C₆ alkyl, C₁-C₆ alkanoyl, phenyl C₁-C₄ alkyl, indazolyl, and phenyl C₁-C₄ alkanoyl.

11. A compound according to claim 10, wherein
R₅ is benzyl or phenethyl, wherein each is optionally substituted with 1, 2, 3, 4, or 5 groups that are independently C₁-C₆ alkyl, —NR₆R₇, —C(O)NR₆R₇—(C₁-C₄ alkyl)-C(O)NR₆R₇, —NR₈R₉, halogen, C₁-C₆ alkoxy, CO₂R, —(C₁-C₄ alkyl)-CO₂R, C₁-C₆ thioalkoxy, amidinooxime, C₁-C₆ alkoxycarbonyl, —(C₁-C₄ alkyl)-C₁-C₆ alkoxycarbonyl, C₁-C₆ hydroxyalkyl, C₁-C₆ dihydroxyalkyl, —(C₁-C₄ alkyl)-CN, CN, phenyl C₁-C₆ alkoxy, OH, C₁-C₄ haloalkyl, C₁-C₄ haloalkoxy, R₆R₇N—(C₁-C₆ alkyl)-, —(C₁-C₄ alkyl)—NR₁₅C(O)R₁₈, amidinooxime, —SO₂(C₁-C₆ alkyl), —O—CH₂—O—, —O—CH₂CH₂—O—, phenyl C₁-C₄ alkoxy, or phenyl; wherein
R₆ and R₇ are independently at each occurrence H, C₁-C₄ alkyl, C₁-C₄ hydroxyalkyl, C₁-C₄ dihydroxyalkyl, C₁-C₄ alkanoyl, or C₁-C₄ alkoxy, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃.

12. A compound according to claim 11, wherein
R₅ is benzyl or phenethyl, each of which is unsubstituted or substituted with 1, 2, 3, 4, or 5 groups that are independently CN, halogen, C₁-C₄ alkoxy, CF₃, OCF₃, C₁-C₄ alkyl, —NR₈R₉, —NR₆R₇, R₆R₇N—(C₁-C₆ alkyl)-, or —C(O)NR₆R₇, wherein
R₆ and R₇ are independently at each occurrence H, C₁-C₄ alkyl, C₁-C₄ hydroxyalkyl, C₁-C₄ dihydroxyalkyl, C₁-C₄ alkanoyl, or C₁-C₄ alkoxy, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃.

13. A compound according to claim 4, wherein the R₅ group is of the formula: 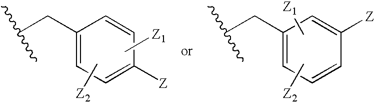
wherein
Z₁ and Z₂ are independently H, halogen, C₁-C₄ alkyl, or CO₂R; and
Z is —C(O)NR₆R₇, —(C₁-C₄ alkyl)-NR₁₅C(O)R₁₈, —NR₆R₇, R₆R₇N—(C₁-C₆ alkyl)-, —NR₈R₉, C₁-C₆ hydroxyalkyl, C₁-C₆ dihydroxyalkyl, C₁-C₆ alkyl, CO₂R, or halogen; wherein
R₆ and R₇ at each occurrence are independently H, OH, C₁-C₆ alkyl, amino C₁-C₄ alkyl, NH(C₁-C₆ alkyl)alkyl, N(C₁-C₆ alkyl)(C₁-C₆ alkyl) C₁-C₆ alkyl, C₁-C₆ hydroxyalkyl, C₁-C₆ dihydroxyalkyl, C₁-C₆ alkoxy C₁-C₆ alkyl, or —SO₂(C₁-C₆ alkyl) each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃; or
R₆, R₇, and the nitrogen to which they are attached form a piperidinyl, pyrrolidinyl, piperazinyl, or a morpholinyl, thiomorpholinyl, ring optionally substituted with 1 or 2 groups that are independently alkyl, hydroxy, hydroxy C₁-C₄ alkyl, C₁-C₄ dihydroxyalkyl, or halogen; and
R₁₈ is C₁-C₆ alkyl optionally substituted with —O—(C₂-C₆ alkanoyl, C₁-C₆ hydroxyalkyl, C₁-C₄ dihydroxyalkyl, C₁-C₆ alkoxy, C₁-C₆ alkoxy C₁-C₆ alkyl; amino C₁-C₆ alkyl, mono or dialkylamino C₁-C₆ alkyl.

14. A compound according to claim 1, wherein
R₅ is pyrazolyl(C₁-C₆ alkyl), imidazolyl(C₁-C₆ alkyl), furanyl (C₁-C₆ alkyl), thienyl (C₁-C₆ alkyl), piperidinyl (C₁-C₆) alkyl, pyrrolidinyl (C₁-C₆) alkyl, imidazolidinyl(C₁-C₆)alkyl, piperazinyl (C₁-C₆)alkyl, pyridyl(C₁-C₆)alkyl, pyrimidyl(C₁-C₆)alkyl, pyridazyl(C₁-C₆)alkyl, pyrazinyl(C₁-C₆) alkyl, isoquinolinyl (C₁-C₆) alkyl, tetrahydroisoquinolinyl (C₁-C₆)alkyl, indolyl (C₁-C₆)alkyl, 1H-indazolyl (C₁-C₆)alkyl, dihydroindolyl (C₁-C₆ alkyl), dihydroindolon-2-yl(C₁-C₆ alkyl), indolinyl(C₁-C₆ alkyl), dihydroisoindolyl(C₁-C₆ alkyl), dihydrobenzimdazolyl(C₁-C₆ alkyl), or dihydrobenzoimidazolonyl(C₁-C₆ alkyl), wherein
each of the above is unsubstituted or substituted with 1, 2, 3, 4, or 5 groups that are independently (C₁-C₆)alkyl, halogen, (C₁-C₆)alkoxy, (C₁-C₆)hydroxyalkyl, C₁-C₆ dihydroxyalkyl, phenyl (C₁-C₆) alkoxy, (C₁-C₆) thioalkoxy, (C₁-C₆) alkoxycarbonyl, phenyl(C₁-C₆)alkoxycarbonyl, OH, CO₂R, CN, amidinooxime, —NR₈R₉, —NR₆R₇, R₆R₇N—(C₁-C₆ alkyl)-, —C(O)NR₆R₇, —(C₁-C₄ alkyl)-C(O)NR₆R₇, amidino, piperazinyl, morpholinyl, —SO₂ (C₁-C₆) alkyl, —SO₂NH₂, —SO₂NH(C₁-C₆)alkyl, —SO₂N(C₁-C₆)alkyl (C₁-C₆)alkyl, (C₁-C₄)haloalkyl, —(C₁-C₄ alkyl) —NR₁₅C(O)NR₁₆R₁₇, —(C₁-C₄ alkyl)—NR₁₅C(O)R₁₈, —O—CH₂—O, —O—CH₂CH₂—O—, or (C₁-C₄)haloalkoxy; wherein
R₆ and R₇ are independently at each occurrence H, (C₁-C₆)alkyl, (C₁-C₆)alkoxy, (C₁-C₆)alkoxy(C₁-C₆) alkyl, (C₁-C₆) alkoxycarbonyl, (C₁-C₆)hydroxyalkyl, C₁-C₆ dihydroxyalkyl, —(C₁-C₄) alkyl-CO₂—(C₁-C₆)alkyl, (C₁-C₆)alkanoyl, phenyl (C₁-C₆)alkyl, phenyl(C₁-C₆)alkoxy, or phenyl(C₁-C₆)alkanoyl, wherein each of the above is unsubstituted or substituted with 1, 2, or 3 groups that are independently, halogen, (C₁-C₄)alkoxy, OH, SH, C₃-C₆ cycloalkyl, NH₂, NH(C₁-C₆ alkyl), N(C₁-C₆ alkyl) (C₁-C₆ alkyl), (C₁-C₄)alkyl, CF₃ or OCF₃; or
R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, hydroxy, hydroxy C₁-C₄ alkyl, C₁-C₄ dihydroxyalkyl, or halogen; and
R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl,
provided that R₆ and R₇ are not simultaneously OH;
provided that R₆ and R₇ are not simultaneously —SO₂(C₁-C₆ alkyl).

15. A compound according to claim 14, wherein
R₅ is thienyl(C₁-C₆ alkyl), pyrimidyl(C₁-C₆)alkyl, pyrazolyl(C₁-C₆ alkyl), indolyl (C₁-C₆ alkyl), dihydroindolyl (C₁-C₆ alkyl), dihydroisoindolyl(C₁-C₆ alkyl), dihydroindolon-2-yl(C₁-C₆ alkyl), pyridinyl(C₁-C₆ alkyl), piperazinyl(C₁-C₆ alkyl), or pyrazinyl(C₁-C₆ alkyl) each of which is optionally substituted with 1, 2, or 3 groups that are independently C₁-C₄ alkyl, C₁-C₄ hydroxyalkyl, C₁-C₄ dihydroxyalkyl, halogen, —C(O)NR₆R₇, —(C₁-C₄ alkyl)-C(O)NR₆R₇, C₁-C₆ alkoxycarbonyl, —NR₆R₇, R₆R₇N—(C₁-C₆ alkyl)-, haloalkyl, C₁-C₆ alkanoyl,
R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy; or
R₆, R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen.

16. A compound according to claim 15, wherein
R₅ is of the formula: 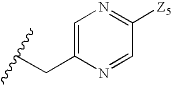
wherein
Z₅ is C₁-C₄ alkyl, C₁-C₄ hydroxyalkyl, C₁-C₄ dihydroxyalkyl, halogen, —C(O)NR₆R₇, —(C₁-C₄ alkyl)-C(O)NR₆R₇, C₁-C₆ alkoxycarbonyl, R₆R₇N—(C₁-C₆ alkyl)-, —NR₆R₇, CF₃, or C₁-C₆ alkanoyl, wherein
R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy; or
R₆, R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen.

17. A compound according to claim 15, wherein
R₅ is of the formula: 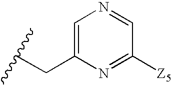
wherein
Z₅ is C₁-C₄ alkyl, C₁-C₄ hydroxyalkyl, C₁-C₄ dihydroxyalkyl, halogen, —C(O)NR₆R₇, —(C₁-C₄ alkyl)-C(O)NR₆R₇, C₁-C₆ alkoxycarbonyl, R₆R₇N—(C₁-C₆ alkyl)-, —NR₆R₇, CF₃, or C₁-C₆ alkanoyl, wherein
R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy; or
R₆, R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen.

18. A compound according to either claim 16 or 17, wherein
Z₅ is C₁-C₄ alkyl, C₁-C₄ hydroxyalkyl, C₁-C₄ dihydroxyalkyl, halogen, C₁-C₆ alkoxycarbonyl, CF₃, or C₁-C₆ alkanoyl.

19. A compound according to either claim 16 or 17, wherein
Z₅ is —C(O)NR₆R₇, —(C₁-C₄ alkyl)-C(O)NR₆R₇, R₆R₇N—(C₁-C₆ alkyl)-, or —NR₆R₇, CF₃, or C₁-C₄ alkanoyl, wherein
R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy; or
R₆, R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen.

20. A compound according to claim 19, wherein
Z₅ is —C(O)NR₆R₇, —(C₁-C₄ alkyl)-C(O)NR₆R₇, R₆R₇N—(C₁-C₆ alkyl)-, or —NR₆R₇, wherein
R₆ and R₇ at each occurrence are independently H, C₁-C₆ alkyl optionally substituted with 1, 2, or 3 groups that are independently C₁-C₄ alkoxycarbonyl, halogen, cyclopropyl, OH, SH, or C₁-C₄ alkoxy.

21. A compound according to claim 15, wherein
R₅ is of the formula: 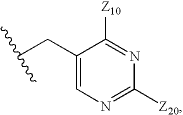
wherein
Z₁₀ is H or methyl; and
Z₂₀ is hydroxy(C₁-C₄)alkyl, C₁-C₄ dihydroxyalkyl, OH, halogen, haloalkyl, (C₁-C₄)alkyl, OCF₃, —NR₆R₇, R₆R₇N—(C₁-C₆ alkyl)-, or —C(O)NR₆R₇, wherein
R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy.

22. A compound according to claim 15, wherein
R₅ is of the formula: 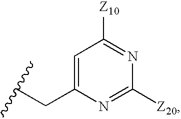
wherein
Z₁₀ is H or methyl; and
Z₂₀ is hydroxy(C₁-C₄)alkyl, C₁-C₄ dihydroxyalkyl, OH, halogen, CF₃, (C₁-C₄)alkyl, OCF₃, —NR₆R₇, R₆R₇N—(C₁-C₆ alkyl)-, or —C(O)NR₆R₇, wherein
R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy.

23. A compound according to claim 4, wherein
R₅ is phenyl, which is optionally substituted with 1, 2, 3, 4, or 5 groups that are independently C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, dihydroxyalkyl, halogen, C₁-C₄ alkoxy, CO₂R, OH, C₁-C₆ alkoxycarbonyl, CF₃, —(C₁-C₄ alkyl)-NR₁₅C(O)NR₁₆R₁₇, —(C₁-C₄ alkyl)-NR₁₅C(O)R₁₈; wherein
R₁₅ is H or C₁-C₆ alkyl;
R₁₆ and R₁₇ are independently H or C₁-C₆ alkyl; or
R₁₆, R₁₇, and the nitrogen to which they are attached form a morpholinyl ring; and
R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl.

24. A compound according to claim 23, wherein
R₅ is of the formula: 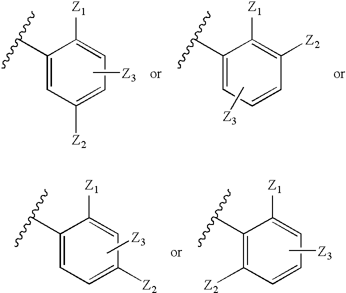
Z₁ is H, halogen, C₁-C₄ alkyl, C₁-C₄ haloalkyl, C₁-C₄ hydroxyalkyl, C₁-C₄ dihydroxyalkyl, or C₁-C₄ alkoxy; and
Z₂ is C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, OH, C₁-C₆ alkoxycarbonyl, or C₁-C₄ haloalkyl;
Z₃ is H, C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, OH, C₁-C₆ alkoxycarbonyl, or C₁-C₄ haloalkyl;
wherein
R₆ and R₇ at each occurrence are independently H, OH, C₁-C₆ alkyl, amino C₁-C₄ alkyl, NH(C₁-C₆ alkyl)alkyl, N(C₁-C₆ alkyl) (C₁-C₆ alkyl) C₁-C₆ alkyl, C₁-C₆ hydroxyalkyl, C₁-C₆ dihydroxyalkyl, C₁-C₆ alkoxy C₁-C₆ alkyl, —SO₂(C₁-C₆ alkyl), —SO₂NH₂, —SO₂NH(C₁-C₆ alkyl), —SO₂N(C₁-C₆ alkyl)(C₁-C₆ alkyl), or C₁-C₆ alkanoyl, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃;
provided that at least one of Z₁, Z₂, and Z₃ is not hydrogen.

25. A compound according to claim 24, wherein
R₅ is of the formula: 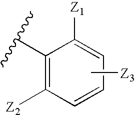
wherein
Z₁ is H, halogen, C₁-C₄ alkyl, C₁-C₄ haloalkyl, C₁-C₄ hydroxyalkyl, C₁-C₄ dihydroxyalkyl, or C₁-C₄ alkoxy; and
Z₂ is C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, OH, C₁-C₆ alkoxycarbonyl, or C₁-C₄ haloalkyl;
Z₃ is H, C₁-C₄ alkyl, —C(O)NR₆R₇, —(C_{1-C}₄ alkyl)-C(O)NR₆R₇, —NR₆R₇, NR₆R₇ (C₁-C₆ alkyl), C₁-C₆ hydroxyalkyl, C₁-C₆ dihydroxyalkyl, halogen, C₁-C₄ alkoxy, CO₂R, OH, C₁-C₆ alkoxycarbonyl, or C₁-C₄ haloalkyl, wherein
R₆ and R₇ at each occurrence are independently H, OH, C₁-C₆ alkyl, amino C₁-C₄ alkyl, NH(C₁-C₆ alkyl)alkyl, N(C₁-C₆ alkyl) (C₁-C₆ alkyl) C₁-C₆ alkyl, C₁-C₆ hydroxyalkyl, C₁-C₆ dihydroxyalkyl, C₁-C₆ alkoxy C₁-C₆ alkyl, —SO₂(C₁-C₆ alkyl), —SO₂NH₂, —SO₂NH(C₁-C₆ alkyl), —SO₂N(C₁-C₆ alkyl) (C₁-C₆ alkyl), or C₁-C₆ alkanoyl, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃;
provided that at least one of Z₁, Z₂, and Z₃ is not hydrogen.

26. A compound according to claim 24, wherein
R₅ is of the formula: 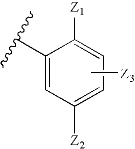
wherein
Z₁ is H, halogen, C₁-C₄ alkyl, C₁-C₄ haloalkyl, C₁-C₄ hydroxyalkyl, C₁-C₄ dihydroxyalkyl, or C₁-C₄ alkoxy; and
Z₂ is C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, OH, C₁-C₆ alkoxycarbonyl, or C₁-C₄ haloalkyl;
Z₃ is H, C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, OH, C₁-C₆ alkoxycarbonyl, or C₁-C₄ haloalkyl, wherein
R₆ and R₇ at each occurrence are independently H, OH, C₁-C₆ alkyl, amino C₁-C₄ alkyl, NH(C₁-C₆ alkyl)alkyl, N(C₁-C₆ alkyl) (C₁-C₆ alkyl) C₁-C₆ alkyl, C₁-C₆ hydroxyalkyl, C₁-C₆ dihydroxyalkyl, C₁-C₆ alkoxy C₁-C₆ alkyl, —SO₂(C₁-C₆ alkyl), —SO₂NH₂, —SO₂NH(C₁-C₆ alkyl), —SO₂N(C₁-C₆ alkyl) (C₁-C₆ alkyl), or C₁-C₆ alkanoyl, each of which is optionally substituted with 1, 2, or 3 groups that are independently halogen, OH, SH, C₃-C₆ cycloalkyl, C₁-C₄ alkoxy, C₁-C₄ alkyl, OH, CF₃, or OCF₃;
provided that at least one of Z₁, Z₂, and Z₃ is not hydrogen.

27. A compound according to claim 23, wherein
R₅ is either 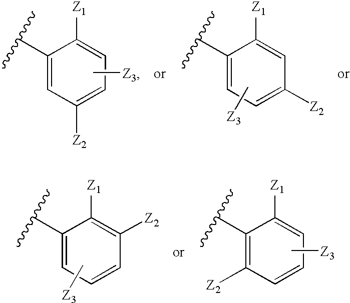
wherein
Z₁ is H, halogen, C₁-C₄ alkyl, C₁-C₄ haloalkyl, C₁-C₄ hydroxyalkyl, C₁-C₄ dihydroxyalkyl, or C₁-C₄ alkoxy; and
Z₂ is C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, C₁-C₆ alkoxycarbonyl, —(C₁-C₄ alkyl)-NR₁₅C(O)NR₁₆R₁₇, or —(C₁-C₄ alkyl) —NR₁₅c(o)R₁₈;
Z₃ is H, C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, C₁-C₆ alkoxycarbonyl, —(C₁-C₄ alkyl)-NR₁₅C(O)NR₁₆R₁₇, or —(C₁-C₄ alkyl) —NR₁₅C(O)R₁₈;
R₆, R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen;
R₁₅ is H or C₁-C₆ alkyl;
R₁₆ and R₁₇ are independently H or C₁-C₆ alkyl; or
R₁₆, R₁₇, and the nitrogen to which they are attached form a morpholinyl ring;
R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl;
provided that at least one of Z₁, Z₂, and Z₃ is not hydrogen.

28. A compound according to claim 27, wherein
R₅ is of the formula: 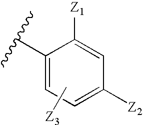
Z₁ is H, halogen, C₁-C₄ alkyl, C₁-C₄ haloalkyl, C₁-C₄ hydroxyalkyl, C₁-C₄ dihydroxyalkyl, or C₁-C₄ alkoxy; and
Z₂ is C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, C₁-C₆ alkoxycarbonyl, —(C₁-C₄ alkyl)-NR₁₅C(O)NR₁₆R₁₇, or —(C₁-C₄ alkyl) —NR₁₅C(O)R₁₈;
Z₃ is H, C₁-C₄ alkyl, —C(O)NR₆R₇, —(C_{1-C}₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, C₁-C₆ alkoxycarbonyl, —(C₁-C₄ alkyl)-NR₁₅C(O)NR₁₆R₁₇, or —(C₁-C₄ alkyl) —NR₁₅C(O)R₁₈;
R₆, R₇, and the nitrogen to which they are attached form a piperidinyl, pyrrolidinyl, piperazinyl, or a morpholinyl ring optionally substituted with 1 or 2 groups that are independently alkyl, hydroxy, hydroxy C₁-C₄ alkyl, C₁-C₄ dihydroxyalkyl, or halogen;
R₁₅ is H or C₁-C₆ alkyl;
R₁₆ and R₁₇ are independently H or C₁-C₆ alkyl; or
R₁₆, R₁₇, and the nitrogen to which they are attached form a morpholinyl ring;
R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl;
provided that at least one of Z₁, Z₂, and Z₃ is not hydrogen.

29. A compound according to claim 27, wherein
R₅ is of the formula: 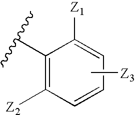
wherein
Z₁ is H, halogen, C₁-C₄ alkyl C₁-C₄ haloalkyl, C₁-C₄ hydroxyalkyl, C₁-C₄ dihydroxyalkyl, or C₁-C₄ alkoxy; and
Z₂ is C₃-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, C₁-C₆ alkoxycarbonyl, —(C₁-C₄ alkyl)-NR₁₅C(O)NR₁₆R₁₇, or —(C₁-C₄ alkyl) —NR₁₅C(O)R₁₈;
Z₃ is H, C₁-C₄ alkyl, —C(O)NR₆R₇, —(C₁-C₄ alkyl)-C(O)NR₆R₇, —NR₆R₇, NR₆R₇(C₁-C₆ alkyl), C₁-C₆ hydroxyalkyl, C₁-C₆ dihydroxyalkyl, halogen, C₁-C₄ alkoxy, CO₂R, C₁-C₆ alkoxycarbonyl, —(C₁-C₄ alkyl)-NR₁₅C(O)NR₁₆R₁₇, or —(C₁-C₄ alkyl) —NR₁₅C(O)R₁₈;
R₆, R₇, and the nitrogen to which they are attached form a piperidinyl, pyrrolidinyl, piperazinyl, or a morpholinyl ring, each of which is optionally substituted with 1 or 2 groups that are independently alkyl, hydroxy, hydroxy C₁-C₄ alkyl, C₁-C₄ dihydroxyalkyl, or halogen;
R₁₅ is H or C₁-C₆ alkyl;
R₁₆ and R₁₇ are independently H or C₁-C₆ alkyl; or
R₁₆, R₁₇, and the nitrogen to which they are attached form a morpholinyl ring;
R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl;
provided that at least one of Z₁, Z₂, and Z₃ is not hydrogen.

30. A compound of the formula 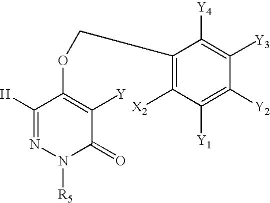
or pharmaceutically acceptable salts thereof, wherein
R₅ is 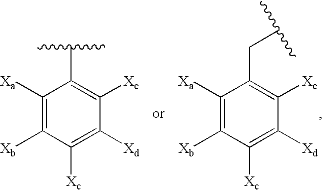
wherein
X₂, Xₐ, X_{b}, X_{c}, X_{d}, and Xₑ are independently selected from —C(O)NR₆R₇, —NR₆R₇, hydroxy(C₁-C₄)alkyl, C₁-C₄ dihydroxyalkyl, H, OH, halogen, haloalkyl, alkoxy, alkyl, haloalkoxy, heteroaryl, heterocycloalkyl, C₃-C₇ cycloalkyl, R₆R₇N—(C₁-C₆ alkyl)-, —CO₂—(C₁-C₆)alkyl, —N(R)C(O)NR₆R₇, —N(R)C(O)—(C₁-C₆)alkoxy, CO₂R—(C₁-C₆ alkyl)-, or —SO₂NR₆R₇; wherein the heteroaryl and heterocycloalkyl groups are optionally substituted with —NR₆R₇, —C(O)NR₆R₇, R₆R₇N—(C₁-C₆ alkyl)-, C₁-C₆ alkyl, C₁-C₆ alkoxy, or halogen; or
R₅ is heteroaryl or heteroarylalkyl, wherein the heteroaryl and heteroaryl groups are optionally substituted with 1, 2, 3, or 4 groups that are independently —C(O)NR₆R₇, —NR₆R₇, hydroxy(C₁-C₄)alkyl, C₁-C₄ dihydroxyalkyl, H, OH, halogen, haloalkyl, alkyl, haloalkoxy, R₆R₇N—(C₁-C₆ alkyl)-, —CO₂—(C₁-C₆)alkyl, —N(R)C(O)NR₆R₇, or —N(R)C(O)—(C₁-C₆)alkoxy; wherein
R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₄ dihydroxyalkyl, C₁-C₆ thiohydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, SH, NH₂, NH(alkyl), N(alkyl)(alkyl), —O—C₁-C₄ alkanoyl, C₁-C₄ alkyl, CF₃, or OCF₃; or
R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen;
R at each occurrence is independently H or C₁-C₆ alkyl; and
Y, Y₁, Y₂, Y₃, and Y₄ are independently selected from H, halogen, alkyl, carboxaldehyde, hydroxyalkyl, dihydroxyalkyl, alkenyl, alkynyl, CN, alkanoyl, alkoxy, alkoxyalkyl, haloalkyl, and carboxyl.

31. A compound according to claim 30, wherein
R₅ is

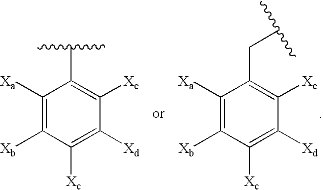

32. A compound according to claim 31, wherein Y₂, Y₄, and Y are independently halogen; and
Y₁ and Y₃ are both hydrogen.

33. A compound according to claim 32, wherein
R₅ is 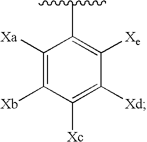
X₂ is H, methyl, NR₆R₇, R₆R₇N—(C₁-C₆ alkyl)-, —C(O)NR₆R₇, C₁-C₆ hydroxyalkyl, C₁-C₆ dihydroxyalkyl, or —(C₁-C₄ alkyl)-morpholinyl; and
Xₐ and Xₑ are independently halogen, NH₂, NH(C₁-C₆ alkyl), N(C₁-C₆ alkyl) (C₁-C₆ alkyl), methyl, or hydrogen; provided that one of Xₐ and Xₑ is not hydrogen.

34. A compound according to claim 33, wherein
one of X_{b} and X_{c} is hydrogen and the other is —NR₆R₇, R₆R₇N—(C₁-C₆ alkyl)-, —C(O)NR₆R₇, —SO₂NR₆R₇, or halogen; where
R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, SH, NH₂, NH(alkyl), N(alkyl)(alkyl), —O—C₁-C₄ alkanoyl, C₁-C₄ alkyl, CF₃, or OCF₃; or
R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen.

35. A compound according to claim 34, wherein
R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄) alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, NH₂, NH(alkyl), N(alkyl) (alkyl), —O—C₁-C₄ alkanoyl, C₁-C₄ alkyl, CF₃, or OCF₃.

36. A compound according to claim 35, wherein
Xₐ is hydrogen, methyl, fluorine, or chlorine;
X_{c} and X_{d} are both hydrogen;
X_{b} is —NR₆R₇, R₆R₇N—(C₁-C₆ alkyl)-, —C(O)NR₆R₇; wherein
R₆ and R₇ are independently at each occurrence H, C₁-C₆ alkyl, C₁-C₆ hydroxyalkyl, C₁-C₄ dihydroxyalkyl, C₁-C₆ alkoxy, C₁-C₆ alkoxy C₁-C₆ alkyl, or C₁-C₆ alkanoyl, wherein each of the above is optionally substituted with 1, 2, or 3 groups that are independently NH₂, NH(C₁-C₆ alkyl), N(C₁-C₆ alkyl)(C₁-C₆ alkyl), OH, SH, halogen, or C₃-C₆ cycloalkyl.

37. A compound according to claim 32, wherein
R₅ is 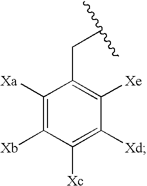
Xₐ is H, fluoro, chloro, or methyl;
Xₑ is hydrogen, halogen, or methyl; and
X_{b} is H;
X_{d} is H or halogen;

38. A compound according to claim 37, wherein
X_{c} is —SO₂NR₆R₇, or halogen; wherein
R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, SH, NH₂, NH(alkyl), N(alkyl)(alkyl), —O—C₁-C₄ alkanoyl, C₁-C₄ alkyl, CF₃, or OCF₃; or
R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen; or
X_{c} is fluoro, chloro, —NH₂, —NH(C₁-C₆ alkyl), —N(C₁-C₆ alkyl)(C₁-C₆ alkyl), —SO₂NH₂, —SO₂NH(C₁-C₆ alkyl), —SO₂N(C₁-C₆ alkyl) (C₁-C₆ alkyl), or piperazinyl, wherein the piperazinyl group is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen.

39. A compound according to claim 37, wherein
X_{c} is —C(O)NR₆R₇, —(C₁-C₆ alkyl)-C(O)NR₆R₇, —NR₆R₇, or R₆R₇N—(C₁-C₆ alkyl)-; wherein
R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, —NH₂, —NH(alkyl), —N(alkyl) (alkyl), —O—C₁-C₄ alkanoyl, C₁-C₄ alkyl, CF₃, or OCF₃; or
R_{6,} R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen.

40. A compound according to claim 39, wherein
R₆ is hydrogen; and
R₇ is C₁-C₆ alkyl or C₁-C₆ alkanoyl, each of which is optionally substituted with 1, 2, or 3 groups that are independently NH₂, NH(C₁-C₆ alkyl), N(C₁-C₆ alkyl)(C₁-C₆ alkyl), OH, SH, cyclopropyl, or C₁-C₄ alkoxy;

41. A compound according to claim 40, wherein
X_{c} is —C(O)NR₆R₇.

42. A compound according to claim 40, wherein
X_{c} is NR₆R₇, or R₆R₇N—(C₁-C₆ alkyl)-.

43. A compound according to claim 31, wherein
Xₐ is hydrogen;
two of X_{b}, X_{c}, and X_{d} are hydrogen and the other is —C(O)NR₆R₇, —(C₁-C₆ alkyl)-C(O)NR₆R₇, —NR₆R₇, R₆R₇N—(C₁-C₆ alkyl)- or —CO₂—(C₁-C₆)alkyl; wherein
R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, NH₂, NH(alkyl), N(alkyl)(alkyl), —O—C₁-C₄ alkanoyl, C₁-C₄ alkyl, CF₃, or OCF₃; or
R₆, R₇, and the nitrogen to which they are attached form a morpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen; and
Xₑ is hydrogen, methyl, C₁-C₂ alkoxy, or halogen.

44. A compound according to claim 43, wherein
X_{b} is —C(O)NR₆R₇, —(C₁-C₆ alkyl)-C(O)NR₆R₇, —NR₆R₇, or R₆R₇N—(C₁-C₆ alkyl)- wherein
R₆ is hydrogen or C₁-C₄ alkyl;
R₇ is OH, C₁-C₆ alkyl or C₁-C₆ alkanoyl, wherein the alkyl and alkanoyl groups substituted with 1, 2, or 3 groups that are independently NH₂, NH(C₁-C₆ alkyl), N(C₁-C₆ alkyl) (C₁-C₆ alkyl), C₃-C₆ cycloalkyl, OH, or C₁-C₄ alkoxy.

45. A compound according to claim 31, wherein
Xₐ is halogen or methyl;
X_{b} is H, —NR₆R₇, R₆R₇N—(C₁-C₆ alkyl)-, —C(O)NR₆R₇, or —CO₂—(C₁-C₆) alkyl;
X_{c} is —NR₆R₇, R₆R₇N—(C₁-C₆ alkyl)-, —C(O)NR₆R₇, halogen, —CO₂—(C₁-C₆)alkyl, NH₂, NH(C₁-C₆ alkyl), N(C₁-C₆ alkyl)(C₁-C₆ alkyl), —SO₂NH₂, —SO₂NH(C₁-C₆ alkyl), —SO₂N(C₁-C₆ alkyl)(C₁-C₆ alkyl), or piperazinyl, wherein the piperazinyl group is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen;
X_{d} is hydrogen;
Xₑ is H, methyl, NH₂, NH(C₁-C₆ alkyl) or N(C₁-C₆ alkyl) (C₁-C₆ alkyl).

46. A compound according to claim 31, wherein
X₂, Xₐ, X_{b}, X_{c}, X_{d}, and Xₑ are independently selected from H, OH, halogen, CF₃, alkyl, OCF₃, pyridyl, pyridazinyl, pyrimidyl, pyrazinyl, thienyl, furyl, pyrrolyl, piperidinyl, piperazinyl, or C₃-C₇ cycloalkyl, wherein each of the above is optionally substituted with —NR₆R₇, —C(O)NR₆R₇, R₆R₇N—(C₁-C₆ alkyl)-, C₁-C₆ alkyl, C₁-C₆ alkoxy, or halogen.

47. A compound according to claim 30, wherein
R₅ is a heteroaryl or heteroarylalkyl group, where each heteroaryl is pyridyl, pyridazinyl, pyrimidinyl, pyrazinyl, pyrazolyl, imidazolyl, dihydroindolyl, dihydroisoindolyl, indolon-2-yl, quinolinyl, isoquinolinyl, tetrahydroisoquinolinyl, dihydroisoquinolinyl, or indolyl, each of which is optionally substituted with 1, 2, 3, or 4 groups that are independently —C(O)NR₆R₇, —NR₆R₇, hydroxy(C₁-C₄)alkyl, C₁-C₄ dihydroxyalkyl, hydrogen, hydroxy, halogen, haloalkyl, alkyl, haloalkoxy, R₆R₇N—(C₁-C₆ alkyl)-, —CO₂—(C₁-C₆)alkyl, —N(R)C(O)NR₆R₇, or —N(R)C(O)—(C₁-C₆)alkoxy; wherein
R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, C₁-C₆ thiohydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, SH, NH₂, NH(alkyl), N(alkyl)(alkyl), —O—C₁-C₄ alkanoyl, C₁-C₄ alkyl, CF₃, or OCF

48. A compound according to claim 47, wherein
Y₂, Y₄, and Y are independently halogen; and
Y₁ and Y₃ are both hydrogen.

49. A compound according to claim 48, wherein
X₂ is H, methyl, —NR₆R₇, R₆R₇N—(C₁-C₆ alkyl)-, —C(O)NR₆R₇, C₁-C₆ hydroxyalkyl, C₁-C₆ dihydroxyalkyl, or —(C₁-C₄ alkyl)-morpholinyl.

50. A compound according to claim 49, wherein
R₅ is pyridyl C₁-C₆ alkyl, pyrimidinyl C₁-C₆ alkyl, or pyrazinyl C₁-C₆ alkyl, each of which is optionally substituted with 1, 2, or 3 groups that are independently hydroxy(C₁-C₄) alkyl, C₁-C₄ dihydroxyalkyl, OH, halogen, CF₃, (C₁-C₄)alkyl, OCF₃, —NR₆R₇, R₆R₇N—(C₁-C₆ alkyl)-, or —C(O)NR₆R₇.

51. A compound according to claim 50, wherein
R₅ is of the formula: 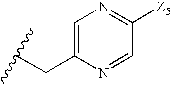
wherein
Z₅ is hydroxy(C₁-C₄)alkyl, C₁-C₄ dihydroxyalkyl, OH, halogen, CF₃, (C₁-C₄)alkyl, OCF₃, —NR₆R₇, R₆R₇N—(C₁-C₆ alkyl)-, or —C(O)NR₆R₇, wherein
R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy.

52. A compound according to claim 50, wherein
R₅ is of the formula: 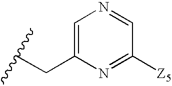
wherein
Z₅ is hydroxy(C₁-C₄)alkyl, C₁-C₄ dihydroxyalkyl, OH, halogen, CF₃, (C₁-C₄)alkyl, OCF₃, —NR₆R₇, R₆R₇N—(C₁-C₆ alkyl)-, or —C(O)NR₆R₇, wherein
R₆ and R₇ at each occurrence are independently H, C₁-C₆ alkyl optionally substituted with 1, 2, or 3 groups that are independently C₁-C₄ alkoxycarbonyl, halogen, C₃-C₆ cycloalkyl, OH, SH, or C₁-C₄ alkoxy.

53. A compound according to claim 50, wherein
R₅ is of the formula: 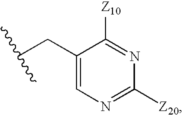
wherein
Z₁₀ is H or methyl; and
Z₂₀ is hydroxy(C₁-C₄)alkyl, C₁-C₄ dihydroxyalkyl, OH, halogen, CF₃, (C₁-C₄) alkyl, OCF₃, —NR₆R₇, R₆R₇N—(C₁-C₆ alkyl)-, or —C(O)NR₆R_{7,} wherein
R₆ and R₇ at each occurrence are independently H, C₁-C₆ alkyl or C₁-C₆ alkanoyl, wherein the alkyl and alkanoyl groups are optionally substituted with 1, 2, or 3 groups that are independently NH₂, NH(C₁-C₆ alkyl), N(C₁-C₆ alkyl)(C₁-C₆ alkyl), C₃-C₆ cycloalkyl, OH, or C₁-C₄ alkoxy.

54. A method of treating a TNF mediated disorder, a p38 kinase mediated disorder, inflammation and/or arthritis in a subject, the method comprising treating a subject having or susceptible to such disorder or condition with a compound of the formula: 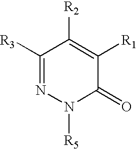
or a pharmaceutically acceptable salt thereof, wherein
R₁ is H, halogen, NO₂, alkyl, carboxaldehyde, hydroxyalkyl, dihydroxyalkyl, arylalkoxy, arylalkyl, alkenyl, alkynyl, arylalkynyl, —CN, aryl, alkanoyl, alkoxy, alkoxyalkyl, haloalkyl, haloalkoxy, carboxyl, aryloxy(C₁-C₆)alkyl, or arylalkanoyl,
wherein the aryl portion of arylalkoxy, arylalkyl, and arylalkanoyl is unsubstituted or substituted with 1, 2, 3, 4, or 5 groups that are independently halogen, C₁-C₄ alkyl, C₁-C₄ alkoxy, nitro, CN, haloalkyl, haloalkoxy or CO₂R;
wherein the alkyl portion of the alkyl, hydroxyalkyl, dihydroxyalkyl, arylalkoxy, aryloxy(C₁-C₆) alkyl, arylalkyl, alkanoyl, alkoxy, alkoxyalkyl and arylalkanoyl groups is unsubstituted or substituted with 1, 2, or 3 groups that are independently halogen, C₁-C₄ alkoxy, C₁-C₄ alkoxycarbonyl, or C₃-C₇ cycloalkyl;
R₂ is H, OH, halogen, —OSO₂—(C₁-C₆) alkyl, —OSO₂-aryl, arylalkoxy, arylalkylthio, arylamino (C₁-C₆) alkyl, arylalkylamino, heteroarylalkoxy, aryloxy, arylthio, arylthioalkoxy, arylalkynyl, alkoxy, aryloxy(C₁-C₆)alkyl, alkyl, alkynyl, —OC(O)NH(CH₂)aryl, —OC(O)N(alkyl)(CH₂)ₙaryl, alkoxyalkoxy, dialkylamino, alkyl, alkoxy, aryl, arylalkyl, heteroaryl, heteroarylalkyl, arylalkenyl, heterocycloalkyl, heterocycloalkylalkyl, alkoxyalkoxy, NR₈R₉, dialkylamino, or CO₂R, wherein
n is 0, 1, 2, 3, 4, 5 or 6;
each of which groups is unsubstituted or substituted with 1, 2, 3, 4, or 5 groups that are independently halogen, —(C₁-C₆)alkyl-N(R) —CO₂R₃₀, haloalkyl, heteroaryl, heteroarylalkyl, —(C₁-C₆alkyl)-C(O)—NR₆R₇, —NR₆R₇, R₆R₇N—(C₁-C₆ alkyl)-, —C(O)NR₆R₇, —(C₁-C₄ alkyl)-NRC(O)NR₁₆R₇, —(C₁-C₄)alkyl-OSO₂—(C₁-C₆)alkyl, haloalkoxy, alkyl, CN, hydroxyalkyl, dihydroxyalkyl, alkoxy, alkoxycarbonyl, phenyl, —SO₂-phenyl wherein the phenyl and —SO₂-phenyl groups are optionally substituted with 1, 2, or 3 groups that are independently halogen or NO₂, or —OC(O)NR₆R₇, wherein R₁₆ and R₁₇ are independently H or C₁-C₆ alkyl; or
R₁₆, R₁₇ and the nitrogen to which they are attached form a morpholinyl ring;
R₆ and R₇ are independently at each occurrence H, alkyl, hydroxyalkyl, dihydroxyalkyl, alkoxy, alkanoyl, arylalkyl, arylalkoxy, alkoxycarbonyl, —SO₂-alkyl, OH, alkoxy, alkoxyalkyl, arylalkoxycarbonyl, —(C₁-C₄)alkyl-CO₂-alkyl, heteroarylalkyl, or arylalkanoyl, wherein each is unsubstituted or substituted with 1, 2, or 3 groups that are independently, halogen, OH, SH, heterocycloalkyl, heterocycloalkylalkyl, C₃-C₇ cycloalkyl, alkoxy, NH₂, NH(alkyl), N(alkyl)(alkyl), —O-alkanoyl, alkyl, haloalkyl, carboxaldehyde, or haloalkoxy; or
R₆, R₇, and the nitrogen to which they are attached form a morpholinyl, pyrrolidinyl, thiomorpholinyl, thiomorpholinyl S-oxide, thiomorpholinyl S,S-dioxide, piperidinyl, pyrrolidinyl, or piperazinyl ring which is optionally substituted with 1 or 2 groups that are independently C₁-C₄ alkyl, alkoxycarbonyl, C₁-C₄ alkoxy, hydroxyl, hydroxyalkyl, dihydroxyalkyl, or halogen;
R at each occurrence is independently hydrogen or C₁-C₆ alkyl optionally substituted with 1 or 2 groups that are independently OH, SH, halogen, amino, monoalkylamino, dialkylamino or C₃-C₆ cycloalkyl;
R₃₀ is C₁-C₆ alkyl optionally substituted with 1 or 2 groups that are independently OH, SH, halogen, amino, monoalkylamino, dialkylamino or C₃-C₆ cycloalkyl;
each R₈ is independently hydrogen, alkyl, alkanoyl, arylalkyl and arylalkanoyl, wherein each of the above is optionally substituted with 1, 2, 3, 4, or 5 groups that are independently alkyl, alkoxy, alkoxycarbonyl, halogen, or haloalkyl;
each R₉ is hydrogen, alkyl, alkanoyl, arylalkyl, cycloalkyl, arylcycloalkyl, cycloalkylalkyl, heterocycloalkylalkyl, arylheterocycloalkyl, alkenyl, heteroaryl, amino, aminoalkyl, monoalkylaminoalkyl, dialkylaminoalkyl, arylalkanoyl, —SO₂-phenyl, and aryl wherein each of the above is optionally substituted with 1, 2, 3, 4, or 5 groups that are independently alkyl, alkoxy, hydroxy, hydroxyalkyl, amino, —(CH₂)₀₋₄—COOR, alkoxycarbonyl, halogen, or haloalkyl;
R₃ is H, halogen, alkoxycarbonyl, arylalkoxycarbonyl, aryloxycarbonyl, arylalkyl, —OC(O)NH(CH₂)ₙaryl, arylalkoxy, —OC(O)N(alkyl)(CH₂)ₙaryl, aryloxy, arylthio, thioalkoxy, arylthioalkoxy, alkenyl, —COOR, hydroxyalkyl, arylalkylcarbonyl, arylalkoxyalkyl, —NR₆R₇, —C(O)NR₆R₇, NR₆R₇—(C₁-C₆)alkyl, or alkyl, wherein
the aryl portion of arylalkoxycarbonyl, aryloxycarbonyl, arylalkyl, —OC(O)NH(CH₂)ₙaryl, arylalkoxy, —OC(O)N(alkyl)(CH₂)ₙaryl, arylalkoxyalkyl, and arylthioalkoxy, is unsubstituted or substituted with 1, 2, 3, 4, or 5 groups that are independently, halogen, alkoxy, alkyl, haloalkyl, or haloalkoxy, wherein n is 0, 1, 2, 3, 4, 5, or 6; and
R₅ is H, aryl, heteroaryl, arylalkyl, arylthioalkyl, alkyl optionally substituted with 1, 2, or 3 groups that are independently arylalkoxycarbonyl, —NR₈R₉, halogen, —C(O)NR₈R₉, alkoxycarbonyl, C₃-C₇ cycloalkyl, or alkanoyl, alkoxy, alkoxyalkyl optionally substituted with one trimethylsilyl group, amino, alkoxycarbonyl, hydroxyalkyl, dihydroxyalkyl, alkynyl, —SO₂-alkyl, alkoxy optionally substituted with one trimethylsilyl group, heterocycloalkylalkyl, cycloalkyl, cycloalkylalkyl, -alkyl-S-aryl, -alkyl-SO₂-aryl, heteroarylalkyl, heterocycloalkyl, heteroaryl, or alkenyl optionally substituted with alkoxycarbonyl, wherein
each of the above is unsubstituted or substituted with 1, 2, 3, 4, or 5 groups that are independently alkyl, halogen, alkoxy, hydroxyalkyl, dihydroxyalkyl, arylalkoxy, thioalkoxy, alkoxycarbonyl, arylalkoxycarbonyl, CO₂R, CN, OH, hydroxyalkyl, dihydroxyalkyl, amidinooxime, —NR₆R₇, —NR₈R₉, R₆R₇N—(C₁-C₆ alkyl)-, carboxaldehyde, SO₂alkyl, —SO₂H, —SO₂NR₆R₇, alkanoyl wherein the alkyl portion is optionally substituted with OH, halogen or alkoxy, —C(O)NR₆R₇, —(C₁-C₄ alkyl)-C(O)NR₆R₇, amidino, haloalkyl, —(C₁-C₄ alkyl) —NR₁₅C(O)NR₁₆R₁₇, —(C₁-C₄ alkyl) —NR₁₅C(O)R₁₈, —O—CH₂—O, —O—CH₂CH₂—O—, or haloalkoxy; wherein
R₁₅ is H or C₁-C₆ alkyl;
R₁₈ is C₁-C₆ alkyl optionally substituted with —O—(C₂-C₆ alkanoyl, C₁-C₆ hydroxyalkyl, C₁-C₆ dihydroxyalkyl, C₁-C₆ alkoxy, C₁-C₆ alkoxy C₁-C₆ alkyl; amino C₁-C₆ alkyl, mono or dialkylamino C₁-C₆ alkyl,
provided that no more than two of R₁, R₂, and R₅ are simultaneously hydrogen.

55. A method according to claim 54 for treating or preventing inflammation; arthritis, rheumatoid arthritis, spondylarthropathies, gouty arthritis, osteoarthritis, systemic lupus erthematosus, juvenile arthritis; neuroinflammation; pain, neuropathic pain; fever; pulmonary disorders, lung inflammation, adult respiratory distress syndrome, pulmonary sarcoisosis, asthma, silicosis, chronic pulmonary inflammatory disease; cardiovascular disease, arteriosclerosis, myocardial infarction, thrombosis, congestive heart failure, cardiac reperfusion injury; cardiomyopathy; reperfusion injury; renal reperfusion injury; ischemia including stroke and brain ischemia; brain trauma; brain edema; liver disease and nephritis; gastrointestinal conditions, inflammatory bowel disease, Crohn's disease, gastritis, irritable bowel syndrome, ulcerative colitis; ulcerative diseases, gastric ulcers; ophthalmic diseases, retinitis, retinopathies, uveitis, ocular photophobia, acute injury to the eye tissue; ophthalmological conditions, corneal graft rejection, ocular neovascularization, retinal neovascularization, neovascularization following injury or infection, diabetic retinopathy, retrolental fibroplasias, neovascular glaucoma; diabetes; diabetic nephropathy; skin-related conditions, psoriasis, eczema, burns, dermatitis, keloid formation, scar tissue formation, angiogenic disorders; viral and bacterial infections, sepsis, septic shock, gram negative sepsis, malaria, meningitis, opportunistic infections, cachexia secondary to infection or malignancy, cachexia secondary to acquired immune deficiency syndrome (AIDS), AIDS, ARC (AIDS related complex), pneumonia, herpes virus; myalgias due to infection; influenza; endotoxic shock; toxic shock syndrome; autoimmune disease, graft vs. host reaction and allograft rejections; treatment of bone resorption diseases, osteoporosis; multiple sclerosis; disorders of the female reproductive system, endometriosis; hemaginomas, infantile hemagionmas, angiofibroma of the nasopharynx, avascular necrosis of bone; benign and malignant tumors/neoplasia, cancer, colorectal cancer, brain cancer, bone cancer, epithelial call-derived neoplasia (epithelial carcinoma), basal cell carcinoma, adenocarcinoma, gastrointestinal cancer, lip cancer, mouth cancer, esophageal cancer, small bowel cancer, stomach cancer, colon cancer, liver cancer, bladder cancer, pancreas cancer, ovarian cancer, cervical cancer, lung cancer, breast cancer, skin cancer, squamus cell and/or basal cell cancers, prostate cancer, renal cell carcinoma, and other known cancers that affect epithelial cells throughout the body; leukemia; lymphoma; systemic lupus erthrematosis (SLE); angiogenesis including neoplasia; metastasis; central nervous system disorders, central nervous system disorders having an inflammatory or apoptotic component, Alzheimer's disease, Parkinson's disease, Huntington's disease, amyotrophic lateral sclerosis, spinal cord injury, and peripheral neuropathy.

56. A compound according to claim 1 that is:
2-benzyl-4-bromo-5-[(4-fluorobenzyl)oxy]pyridazin-3(2H)-one;
2-benzyl-4-chloro-5-methoxypyridazin-3(2H)-one;
4,5-dibromo-2-(2,6-dichlorophenyl)pyridazin-3(2H)-one;
2-benzyl-4,5-dibromopyridazin-3(2H)-one;
4,5-dibromo-2-phenylpyridazin-3(2H)-one;
2-benzyl-4-bromo-5-[(4-fluorobenzyl)amino]pyridazin-3(2H)-one;
4-bromo-2-(2,6-dichlorophenyl)-5-[(4-fluorobenzyl)oxy]pyridazin-3(2H)-one;
4-bromo-2-(2,6-dichlorophenyl)-5-[(4-fluorobenzyl)amino]pyridazin-3(2H)-one;
2-benzyl-4-bromo-5-(phenethyloxy)pyridazin-3(2H)-one;
2-benzyl-5-(benzyloxy)pyridazin-3(2H)-one;
4-bromo-2-(2,6-dichlorophenyl)-5-[(1-methyl-1-phenylethyl)amino]pyridazin-3(2H)-one;
ethyl{[5-bromo-1-(2,6-dichlorophenyl)-6-oxo-1,6-dihydropyridazin-4-yl]amino}(phenyl)acetate;
4-bromo-5-[(2-chlorobenzyl)amino]-2-(2,6-dichlorophenyl)pyridazin-3(2H)-one;
4-bromo-5-[(3-chlorobenzyl)amino]-2-(2,6-dichlorophenyl)pyridazin-3(2H)-one;
4-bromo-2-(2,6-dichlorophenyl)-5-[(2-phenylethyl)amino]pyridazin-3(2H)-one;
5-[benzyl(methyl)amino]-4-bromo-2-(2,6-dichlorophenyl)pyridazin-3(2H)-one;
4-bromo-2-(3,5-dichloropyridin-4-yl)-5-[(2,4-difluorobenzyl)oxy]pyridazin-3(2H)-one;
4-bromo-2-(2,6-dichlorophenyl)-5-[(2,3,4-trifluorobenzyl)oxy]pyridazin-3(2H)-one;
4-bromo-2-(2,6-dichlorophenyl)-5-[(2,4,6-trifluorobenzyl)oxy]pyridazin-3(2H)-one;
4-bromo-2-(2,6-dichlorophenyl)-5-{[2-(hydroxymethyl)benzyl]oxy}pyridazin-3(2H)-one;
4-bromo-2-(3,5-dichloropyridin-4-yl N-oxide)-5-[(2,4-difluorobenzyl)oxy]pyridazin-3(2H)-one;
2-({[5-bromo-1-(2,6-dichlorophenyl)-6-oxo-1,6-dihydropyridazin-4-yl]oxy}methyl)benzyl methanesulfonate;
4-bromo-2-(2,6-dichlorophenyl)-5-{[2-(2-fluorophenyl)ethyl]amino}pyridazin-3(2H)-one;
2-benzyl-4-bromo-5-(2-phenylethoxy)pyridazin-3(2H)-one;
5-(benzyloxy)-4-bromo-2-phenylpyridazin-3(2H)-one;
5-(benzylamino)-4-bromo-2-(2,6-dichlorophenyl)pyridazin-3(2H)-one;
4-bromo-2-(2,6-dichlorophenyl)-5-(2-phenylethoxy)pyridazin-3(2H)-one;
5-(benzyloxy)-4-bromo-2-(2,6-dichlorophenyl)pyridazin-3(2H)-one;
4-bromo-2-(2,6-dichlorophenyl)-5-[(2-hydroxy-2-phenylethyl)amino]pyridazin-3(2H)-one;
4-bromo-2-(2,6-dichlorophenyl)-5-{[(1R,2S)-2-hydroxy-1-methyl-2-phenylethyl]amino}pyridazin-3(2H)-one;
4-bromo-2-(2,6-dichlorophenyl)-5-{[(1S,2R)-2-hydroxy-1-methyl-2-phenylethyl]amino}pyridazin-3(2H)-one;
5-[(1-benzyl-2-hydroxyethyl)amino]-4-bromo-2-(2,6-dichlorophenyl)pyridazin-3(2H)-one;
4-bromo-2-(2,6-dichlorophenyl)-5-{[(1S)-2-hydroxy-1-phenylethyl]amino}pyridazin-3(2H)-one;
3-[5-bromo-4-[(2,4-difluorobenzyl)oxy]-6-oxopyridazin-1(6H)-yl]-4-methylbenzamide
4-bromo-2-(2,6-dichlorophenyl)-5-[methyl(2-phenylethyl)amino]pyridazin-3(2H)-one;
4-bromo-2-(2,6-dichlorophenyl)-5-[(2-hydroxyethyl)(2-phenylethyl)amino]pyridazin-3(2H)-one;
5-[(2-aminobenzyl)amino]-4-bromo-2-(2,6-dichlorophenyl)pyridazin-3(2H)-one;
3-[5-bromo-4-[(2,4-difluorobenzyl)oxy]-6-oxopyridazin-1(6H)-yl]—N,4-dimethylbenzamide
4-bromo-2-(2,6-dichlorophenyl)-5-[4-(4-fluorophenyl)piperazin-1-yl]pyridazin-3(2H)-one;
4-bromo-2-(2,6-dichlorophenyl)-5-[(2-methoxybenzyl)oxy]pyridazin-3(2H)-one;
4-bromo-2-(2,6-dichlorophenyl)-5-(3-phenylpropoxy)pyridazin-3(2H)-one;
4-bromo-2-(2,6-dichlorophenyl)-5-(2-pyridin-2-ylethoxy)pyridazin-3(2H)-one;
4-bromo-2-(2,6-dichlorophenyl)-5-hydroxypyridazin-3(2H)-one;
4-{[5-bromo-1-(2,6-dichlorophenyl)-6-oxo-1,6-dihydropyridazin-4-yl]amino}-3-(4-chlorophenyl)butanoic acid;
4-bromo-5-{[2-(chloromethyl)benzyl]oxy}-2-(2,6-dichlorophenyl)pyridazin-3(2H)-one;
5-(1-benzylhydrazino)-4-bromo-2-(2,6-dichlorophenyl)pyridazin-3(2H)-one;
4-bromo-2-(2,6-dichlorophenyl)-5-[(2,4-difluorobenzyl)oxy]pyridazin-3(2H)-one;
4-bromo-2-(2,6-dichlorophenyl)-5-[(3,4-difluorobenzyl)oxy]pyridazin-3(2H)-one;
2-(2,6-dichlorophenyl)-5-(2-phenylethoxy)pyridazin-3(2H)-one;
2-(2,6-dichlorophenyl)-4-methyl-5-(2-phenylethoxy)pyridazin-3(2H)-one;
2-(2,6-dichlorophenyl)-4-methoxy-5-(2-phenylethoxy)pyridazin-3(2H)-one;
2-(2,6-dichlorophenyl)-4-isobutyl-5-(2-phenylethoxy)pyridazin-3(2H)-one;
2-(2,6-dichlorophenyl)-4-phenoxy-5-(2-phenylethoxy)pyridazin-3(2H)-one;
4-bromo-5-(2-phenylethoxy)pyridazin-3(2H)-one;
4-bromo-5-(2-phenylethoxy)-2-(pyridin-4-ylmethyl)pyridazin-3(2H)-one;
4-bromo-2-(2-hydroxyethyl)-5-(2-phenylethoxy)pyridazin-3(2H)-one;
4-bromo-5-[(2,4-difluorobenzyl)oxy]pyridazin-3(2H)-one;
4-bromo-5-[(2,4-difluorobenzyl)oxy]-2-(pyridin-4-ylmethyl)pyridazin-3(2H)-one;
4-bromo-5-[(2,4-difluorobenzyl)oxy]-2-[2-(dimethylamino)ethyl]pyridazin-3(2H)-one;
4-bromo-5-[(2,4-difluorobenzyl)oxy]-2-[3-(dimethylamino)propyl]pyridazin-3(2H)-one;
4-bromo-5-[(2,4-difluorobenzyl)oxy]-2-(2-hydroxyethyl)pyridazin-3(2H)-one;
4-bromo-5-[(2,4-difluorobenzyl)oxy]-2-[(2-methyl-1,3-thiazol-4-yl)methyl]pyridazin-3(2H)-one; or a pharmaceutically acceptable salt thereof.

57. A compound of the formula 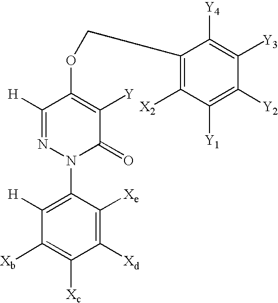
or pharmaceutically acceptable salts thereof, wherein
X₂, X_{b}, X_{c}, X_{d}, and Xₑ are independently selected from —C(O)NR₆R₇, —NR₆R₇, hydroxy (C₁-C₄) alkyl, C₁-C₄ dihydroxyalkyl, H, OH, halogen, haloalkyl, alkyl, alkoxy, haloalkoxy, heteroaryl, heterocycloalkyl, C₃-C₇ cycloalkyl, R₆R₇N—(C₁-C₆ alkyl)-, —CO₂—(C₁-C₆)alkyl, —N(R)C(O)NR₆R₇, —N(R)C(O)—(C₁-C₆)alkoxy, CO₂R—(C₁-C₆ alkyl)-, or —SO₂NR₆R₇; wherein the heteroaryl and heterocycloalkyl groups are optionally substituted with —NR₆R₇, —C(O)NR₆R₇, R₆R₇N—(C₁-C₆ alkyl)-C₁-C6 alkyl, C₁-C₆ alkoxy, or halogen;
R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₄ dihydroxyalkyl, C₁-C₆ thiohydroxyalkyl, —(C₁-C₄) alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, SH, NH₂, NH(alkyl), N(alkyl)(alkyl), —O—C₁-C₄ alkanoyl, C₁-C₄ alkyl, CF₃, or OCF₃; or
R₆, R₇, and the nitrogen to which they are attached form a morpholinyl, thiomorpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen;
R at each occurrence is independently H or C₁-C₆ alkyl; and
Y, Y₁, Y₂, Y₃, and Y₄ are independently selected from H, halogen, alkyl, carboxaldehyde, hydroxyalkyl, dihydroxyalkyl, alkenyl, alkynyl, CN, alkanoyl, alkoxy, alkoxyalkyl, haloalkyl, and carboxyl.

58. A compound according to claim 57, wherein
two of X_{b}, X_{c}, and X_{d} are hydrogen or alkyl, and the other is —C(O)NR₆R₇, —(C₁-C₆ alkyl)-C(O)NR₆R₇, —NR₆R₇, R₆R₇N—(C₁-C₆ alkyl)- or —CO₂—(C₁-C₆)alkyl; wherein
R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄)alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₃-C₆ cycloalkyl, C₁-C₆ alkoxy, piperidinyl C₁-C₆ alkyl, morpholinyl C₁-C₆ alkyl, piperazinyl C₁-C₆ alkyl, OH, NH₂, NH(alkyl), N(alkyl)(alkyl), —O—C₁-C₄ alkanoyl, C₁-C₄ alkyl, CF₃, or OCF₃; or
R₆, R₇, and the nitrogen to which they are attached form a morpholinyl, piperidinyl, pyrrolidinyl, or piperazinyl ring which is optionally substituted with 1 or 2 groups that are independently C₁-C₄ alkyl, C₁-C₄ alkoxy, hydroxy, hydroxy C₁-C₄ alkyl, C₁-C₄ dihydroxyalkyl, or halogen; and
Xₑ is hydrogen, C₁-C₄ alkyl, C₁-C₄ alkoxy, or halogen.

59. A compound according to claim 58, wherein
X_{b} is —C(O)NR₆R₇, —(C₁-C₆ alkyl)-C(O)NR₆R₇, —NR₆R₇, or R₆R₇N—(C₁-C₆ alkyl)- wherein
R₆ and R₇ are independently at each occurrence H, C₁-C₆ alkyl, C₁-C₆ alkoxy, C₁-C₆ alkoxy C₁-C₆ alkyl, C₁-C₆ alkoxycarbonyl, OH, C₁-C₆ hydroxyalkyl, C₁-C₆ dihydroxyalkyl, —(C₁-C₄) alkyl-CO₂-alkyl, pyridyl C₁-C₆ alkyl, C₁-C₆ alkanoyl, benzyl, phenyl C₁-C₆ alkoxy, or phenyl C₁-C₆ alkanoyl, wherein each of the above is unsubstituted or substituted with 1, 2, or 3 groups that are independently, halogen, C₁-C₆ alkoxy, OH, NH₂, NH(alkyl), N(alkyl) (alkyl), —O—C₁-C₄ alkanoyl, C₁-C₄ alkyl, CF₃, or OCF₃.

60. A compound according to claim 59, wherein
X_{b} is —NR₆R₇, or —C(O)NR₆R₇;
X_{c} and X_{d} are both hydrogen or methyl;
Y1 is selected from H, halogen, alkyl, hydroxyalkyl, CN, alkanoyl, alkoxy, alkoxyalkyl, CF₃, and carboxyl; and
Y₂, and Y₄ are independently selected from H, halogen, C₁-C₄ alkyl, C₁-C₄ hydroxyalkyl, C₁-C₄ alkoxy, C₁-C₄ alkoxy C₁-C₄ alkyl, and CF₃.

61. A compound according to claim 60, wherein
Xₑ is C₁-C₄ alkyl, C₁-C₃ alkoxy, or halogen;
Y is methyl or halogen.

62. A compound according to claim 61, wherein
Y₃ is H, halogen, or C₁-C₄ alkyl;
Y₂ and Y₄ are independently H or halogen;
X₂ is selected from H, halogen, R₆R₇N—(C₁-C₆ alkyl)-, —N(R)C(O)NR₆R₇, or —N(R)C(O)—(C₁-C₆)alkoxy;
R₆ is hydrogen or C₁-C₄ alkyl; and
R₇ is H, OH, C₁-C₆ alky, alkoxy, or C₁-C₆ alkanoyl, wherein the alkyl, alkoxy, and alkanoyl groups are optionally substituted with 1, 2, or 3 groups that are independently NH₂, NH(C₁-C₆ alkyl), N(C₁-C₆ alkyl)(C₁-C₆ alkyl), C₃-C₆ cycloalkyl, OH, or C₁-C₄ alkoxy.

63. A compound according to claim 62, wherein
at least one of X_{c} and X_{d} is hydrogen;
Xₑ is halogen, methyl, or ethyl;
Y is methyl, chloro or bromo;
Y₁ is selected from H, halogen, C₁-C₄ alkyl, C₁-C₄ alkoxy, C₁-C₄ alkoxy C₁-C₄ alkyl, and CF₃;
R₇ is H, C₁-C₆ alkyl, C₁-C₆ alkoxy, or C₁-C₆ alkanoyl wherein each of the above is optionally substituted with 1, or 2 groups that are independently OH, SH, or halogen.

64. A compound according to claim 63, wherein
X₂ is selected from H, halogen, or R₆R₇N—(C₁-C₆ alkyl)-;
R₇ is H, C₁-C₆ alkyl, or C₁-C₆ alkanoyl wherein the alkyl portion of each is optionally substituted with 1 or 2 groups that are independently OH, SH, or halogen.

65. A compound according to claim 57, wherein
X_{b} is —NR₆R₇, or —C(O)NR₆R₇;
X_{c} and X_{d} are simultaneously H;
Xₑ is methyl or methoxy;
Y is methyl, Cl or Br;
Y₁ and Y₃ are simultaneously H;
Y₂ and Y₄ are independently H, F, Cl, Me, OMe, —CH₂OH, —CH₂—OCH₃;
X₂ is selected from H, halogen, R₆R₇N—(C₁-C₆ alkyl)-, —N(R)C(O)NR₆R₇, or —N(R)C(O)—(C₁-C₆)alkoxy;
R₆ is hydrogen or C₁-C₄ alkyl optionally substituted with 1, 2, or 3 groups that are independently NH₂, NH(C₁-C₆ alkyl), N(C₁-C₆ alkyl) (C₁-C₆ alkyl), C₃-C₆ cycloalkyl, OH, or C₁-C₄ alkoxy; and
R₇ is H, OH, C₁-C₆ alkyl, alkoxy, or C₁-C₆ alkanoyl, wherein the alkyl portions of each are optionally substituted with 1, 2, or 3 groups that are independently NH₂, NH(C₁-C₆ alkyl), N(C₁-C₆ alkyl) (C₁-C₆ alkyl), C₃-C₆ cycloalkyl, OH, or C₁-C₄ alkoxy.

66. A compound according to claim 65, wherein
X_{b} is —C(O)NR₆R₇;
Xₑ is methyl;
Y₂ and Y₄ are independently H, F, Me, or OMe; and
X₂ is H, halogen, R₆R₇N—(C₁-C₆ alkyl)-, or —N(R)C(O)—(C₁-C₆) alkoxy.

67. A compound according to claim 66, wherein
R₆ and R₇ are independently H or alkyl optionally substituted with 1 or 2 groups that are independently NH₂, NH(C₁-C₆ alkyl), N(C₁-C₆ alkyl)(C₁-C₆ alkyl), C₃-C₆ cycloalkyl, OH, or C₁-C₄ alkoxy.

68. A compound according to claim 66, wherein
R₆ and R₇ are independently H or alkyl optionally substituted with 1 group that is selected from NH₂, NH(C₁-C₄ alkyl), N(C₁-C₄ alkyl) (C₁-C₄ alkyl), OH, or C₁-C₄ alkoxy.

69. A compound according to claim 66, wherein
R₆ and R₇ are both H.

70. A compound according to claim 66, wherein
Y₂ and Y₄ are independently F, Me, or OMe.

71. A compound according to claim 70, wherein
R₆ and R₇ are independently H or C₁-C₆ alkyl.

72. A compound according to claim 71, wherein
X₂ is H.

73. A compound according to claim 72, wherein
at least one of R₆ and R₇ is H.

74. A compound according to claim 73, wherein
Y₂ and Y₄ are simultaneously fluorine.

75. Compounds according to claim 36, wherein
Xₐ is hydrogen;
X_{b} is —NR₆R₇, or —C(O)NR₆R₇;
X_{c} and X_{d} are both hydrogen.

76. A compound according to claim 75, wherein
Xₑ is halogen, NH₂, NH(C₁-C₆ alkyl), N(C₁-C₆ alkyl) (C₁-C₆ alkyl), or methyl.

77. A compound according to claim 76, wherein
Xₑ is halogen or methyl.

78. A compound according to claim 76, wherein
Xₑ is methyl.

79. A compound according to claim 77, wherein
R₆ and R₇ are independently at each occurrence H, C₁-C₆ alkyl, C₁-C₆ hydroxyalkyl, C₁-C₆ alkoxy, or C₁-C₆ alkanoyl, wherein each of the above is optionally substituted with 1 or 2 groups that are independently NH₂, NH(C₁-C₆ alkyl), N(C₁-C₆ alkyl)(C₁-C₆ alkyl), OH, SH, or halogen.

80. A compound according to claim 79, wherein
R₆ is H.

81. Compounds according to claim 44, wherein
R₅ is of the formula:

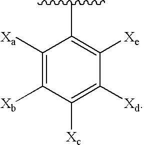

82. A compound according to claim 81, wherein
Xₐ is hydrogen;
X_{b} is —NR₆R₇, or —C(O)NR₆R₇; and
X_{c} and X_{d} are both hydrogen.

83. A compound according to claim 82, wherein
Xₑ is methyl, C₁-C₂ alkoxy, or halogen.

84. A compound according to claim 83, wherein
Xₑ is halogen or methyl.

85. A compound according to claim 84, wherein
Xₑ is methyl.

86. A compound according to claim 84, wherein
R₆ is hydrogen or C₁-C₄ alkyl; and
R₇ is OH, C₁-C₆ alkyl or C₁-C₆ alkanoyl, wherein the alkyl and alkanoyl groups substituted with 1, 2, or 3 groups that are independently NH₂, NH(C₁-C₆ alkyl), N(C₁-C₆ alkyl) (C₁-C₆ alkyl), C₃-C₆ cycloalkyl, OH, or C₁-C₄ alkoxy.
